SEQUENCE LISTING (63 sequences)

SEQ ID NO: 1 - DNA, 930 bp, Arabidopsis thaliana
ctaagtaaaataagataaaacatgttatttgaatttgaatatcgtgggatgcgtatttcggtatttgattaaaggtctggaaaccggagctcctataacccgaataaaaatgcataacatgttcttccccaacgaggcgagcgggtcagggcactagggtcattgcaggcagctcataaagtcatgatcatctaggagatcaaattgtatgtcggccttctcaaaattacctctaagaatctcaaacccaatcatagaacctctaaaaagacaaagtcgtcgctttagaatgggttcggtttttggaaccatatttcacgtcaatttaatgtttagtataatttctgaacaacagaattttggatttatttgcacgtatacaaatatctaattaataaggacgactcgtgactatccttacattaagtttcactgtcgaaataacatagtacaatacttgtcgttaatttccacgtctcaagtctataccgtcatttacggagaaagaacatctctgtttttcatccaaactactattctcactttgtctatatatttaaaattaagtaaaaaagactcaatagtccaataaaatgatgaccaaatgagaagatggttttgtgccagattttaggaaaagtgagtcaaggtttcacatctcaaatttgactgcataatcttcgccattaacaacggcattatatatgtcaagccaattttccatgttgcgtacttttctattgaggtgaaaatatgggtttgttgattaatcaaagagtttgcctaactaatataactacgactttttcagtgaccattccatgtaaactctgcttagtgtttcatttgtcaacaatattgtcgttactcattaaatcaaggaaaaatatacaattgtataattttcttatattttaaaattaattttga

SEQ ID NO: 2 - DNA, 86 bp, Arabidopsis thaliana
ccaaaagaacatctttccttcgaattttctttcattaacatttcttttacttgtctccttgtgtcttcacttcacatcacaacatg

SEQ ID NO: 3 - DNA, 949 bp, Arabidopsis thaliana
actacacccaaaagaacatctttccttcgaattttctttcaattaacatttcttttacttgtctccttgtgtcttcacttcacatcacaacatggctttgaagacagttttcgtagcttttatgattctccttgccatctattcgcaaacgacgtttggggacgatgtgaagtgcgagaatctggatgaaaacacgtgtgccttcgcggtctcgtccactggaaaacgttgcgttttggagaagagcatgaagaggagcgggatcgaggtgtacacatgtcgatcatcggagatagaagctaacaaggtcacaaacattattgaatcggacgagtgcattaaagcgtgtggtctagaccggaaagctttaggtatatcttcggacgcattgttggaatctcagttcacacataaactctgctcggttaaatgcttaaaccaatgtcctaacgtagtcgatctctacttcaaccttgctgctggtgaaggagtgtatttaccaaagctatgtgaatcacaagaagggaagtcaagaagagcaatgtcggaaattaggagctcgggaattgcaatggacactcttgcaccggttggaccagtcatgttgggcgagatagcacctgagccggctacttcaatggacaacatgccttacgtgccggcaccttcaccgtattaattaaggcaagggaaaatggagaggacacgtatgatatcatgagttttcgacgagaataattaagagatttatgtttagttcgacggttttagtattacatcgtttattgcgtccttatatatatgtacttcataaaaacacaccacgacacattaagagatggtgaaagtaggctgcgttctggtgtaacttttacacaagtaacgtcttataatatatatgattcgaataaaatgttgagttttggtgaaaatatataatatgtttctg

SEQ ID NO: 4 - protein, 195 aa, Arabidopsis thaliana
MALKTVFVAFMILLAIYSQTTFGDDVKCENLDENTCAFAVSSTGKRCVLEKSMKRSGIEVYTCRSSEIEANKVTNIIESDECIKACGLDRKALGISSDALLESQFTHKLCSVKCLNQCPNVVDLYFNLAAGEGVYLPKLCESQEGKSRRAMSEIRSSGIAMDTLAPVGPVMLGEIAPEPATSMDNMPYVPAPSPY

SEQ ID NO: 5 - DNA, 963 bp, Arabidopsis thaliana
tatttgtagtgacatattctacaattatcacatttttctcttatgtttcgtagtcgcagatggtcaattttttctataataatttgtccttgaacacaccaaactttagaaacgatgatatataccgtattgtcacgctcacaatgaaacaaacgcgatgaatcgtcatcaccagctaaaagcctaaaacaccatcttagttttcactcagataaaaagattatttgtttccaacctttctattgaattgattagcagtgatgacgtaattagtgatagtttatagtaaaacaaatggaagtggtaataaatttacacaacaaaatatggtaagaatctataaaataagaggttaagagatctcatgttatattaaatgattgaaagaaaaacaaactattggttgatttccatatgtaatagtaagttgtgatgaaagtgatgacgtaattagttgtatttatagtaaaacaaattaaaatggtaaggtaaatttccacaacaaaacttggtaaaaatcttaaaaaaaaaaaaagaggtttagagatcgcatgcgtgtcatcaaaggttctttttcactttaggtctgagtagtgttagactttgattggtgcacgtaagtgtttcgtatcgcgatttaggagaagtacgttttacacgtggacacaatcaacggtcaagatttcgtcgtccagatagaggagcgatacgtcacgccattcaacaatctcctcttcttcattccttcattttgattttgagttttgatctgcccgttcaaaagtctcggtcatctgcccgtaaatataaagatgattatatttatttatatcttctggtgaaagaagctaatataaagcttccatggctaatcttgtttaagcttctcttcttcttctctctcctgtgtctcgttcactagttttttttcgggggagagtgatggagtgtgtttgttgaata

SEQ ID NO: 6 - DNA, 1627 bp, Arabidopsis thaliana
aaagcttccatggctaatcttgtttaagcttctcttcttcttctctctcctgtgtctcgttcactagttttttttcgggggagagtgatggagtgtgtttgttgaatagttttgacgatcacatggctgagatttgttacgagaacgagactatgatgattgaaacgacggcgacggtggtgaagaaggcaacgacgacaacgaggagacgagaacggagctcgtctcaagcagcgagaagaaggagaatggagatccggaggtttaagtttgtttccggcgaacaagaacctgtcttcgtcgacggtgacttacagaggcggaggagaagagaatccaccgtcgcagcctccacctccaccgtgttttacgaaacggcgaaggaagttgtcgtcctatgcgagtctcttagttcaacggttgtggcattgcctgatcctgaagcttatcctaaatacggcgtcgcttcagtctgtggaagaagacgtgaaatggaagacgccgtcgctgtgcatccgtttttttcccgtcatcagacggaatattcatccaccggatttcactattgcggcgtttacgatggccatggctgttcccatgtagcgatgaaatgtagagaaagactacacgagctagtccgtgaagagtttgaagctgatgctgactgggaaaagtcaatggcgcgtagcttcacgcgcatggacatggaggttgttgcgttgaacgccgatggtgcggcaaaatgccggtgcgagcttcagaggccggactgcgacgcggtgggatccactgcggttgtgtctgtccttacgccggagaaaatcatcgtggcgaattgcggtgactcacgtgccgttctctgtcgtaacggcaaagccattgctttatcctccgatcataagccagaccgtccggacgagctagaccggattcaagcagcgggtggtcgtgttatctactgggatggcccacgtgtccttggagtacttgcaatgtcacgagccattggagataattacttgaagccgtatgtaatcagcagaccggaggtaaccgtgacggaccgggccaacggagacgattttcttattctcgcaagtgacggtctttgggacgttgtttcaaacgaaactgcatgtagcgtcgttcgaatgtgtttgagaggaaaagtcaatggtcaagtatcatcatcaccggaaagggaaatgacaggtgtcggcgccgggaatgtggtggttggaggaggagatttgccagataaagcgtgtgaggaggcgtcgctgttgctgacgaggcttgcgttggctagacaaagttcggacaacgtaagtgttgtggtggttgatctacgacgagacacgtagttgtatttgtctctctcgtaatgtttgttgttttttgtcctgagtcatcgacttttgggctttttcttttaaccttttttgctcttcggtgtaagacaacgaagggtttttaatttagcttgactatgggttatgtcagtcactgtgttgaatcgcggtttagatctacaaagattttcaccagtagtgaaaatggtaaaaagccgtgaaatgtgaaagacttgagttcaatttaattttaaatttaatagaatcagttgatc

SEQ ID NO: 7 - protein, 413 aa, Arabidopsis thaliana
MAEICYENETMMIETTATVVKKATTTTRRRERSSSQAARRRRMEIRRFKFVSGEQEPVFVDGDLQRRRRRESTVAASTSTVFYETAKEVVVLCESLSSTVVALPDPEAYPKYGVASVCGRRREMEDAVAVHPFFSRHQTEYSSTGFHYCGVYDGHGCSHVAMKCRERLHELVREEFEADADWEKSMARSFTRMDMEVVALNADGAAKCRCELQRPDCDAVGSTAVVSVLTPEKIIVANCGDSRAVLCRNGKAIALSSDHKPDRPDELDRIQAAGGRVIYWDGPRVLGVLAMSRAIGDNYLKPYVISRPEVTVTDRANGDDFLILASDGLWDVVSNETACSVVRMCLRGKVNGQVSSSPEREMTGVGAGNVVVGGGDLPDKACEEASLLLTRLALARQSSDNVSVVVVDLRRDT

SEQ ID NO: 8 - DNA, 950 bp, Arabidopsis thaliana
aaaattccaattattgtgttactctattcttctaaatttgaacactaatagactatgacatatgagtatataatgtgaagtcttaagatattttcatgtgggagatgaataggccaagttggagtctgcaaacaagaagctcttgagccacgacataagccaagttgatgaccgtaattaatgaaactaaatgtgtgtggttatatattagggacccatggccatatacacaatttttgtttctgtcgatagcatgcgtttatatatatttctaaaaaaactaacatatttactggatttgagttcgaatattgacactaatataaactacgtaccaaactacatatgtttatctatatttgattgatcgaagaattctgaactgttttagaaaatttcaatacacttaacttcatcttacaacggtaaaagaaatcaccactagacaaacaatgcctcataatgtctcgaaccctcaaactcaagagtatacattttactagattagagaatttgatatcctcaagttgccaaagaattggaagcttttgttaccaaacttagaaacagaagaagccacaaaaaaagacaaagggagttaaagattgaagtgatgcatttgtctaagtgtgaaaggtctcaagtctcaactttgaaccataataacattactcacactccctttttttttctttttttttcccaaagtaccctttttaattccctctataacccactcactccattccctctttctgtcactgattcaacacgtggccacactgatgggatccacctttcctcttacccacctcccggtttatataaacccttcacaacacttcatcgctctcaaaccaactctctcttctctcttctctcctctcttctacaagaagaaaaaaaacagagcctttacacatctcaaaatcgaacttactttaaccacc

SEQ ID NO: 9 - DNA, 2310 bp, Arabidopsis thaliana
aaaccaactctctcttctctcttctctcctctcttctacaagaagaaaaaaaacagagcctttacacatctcaaaatcgaacttactttaaccaccaaatactgattgaacacacttgaaaaatggcttctttcacggcaacggctgcggtttctgggagatggcttggtggcaatcatactcagccgccattatcgtcttctcaaagctccgacttgagttattgtagctccttacctatggccagtcgtgtcacacgtaagctcaatgtttcatctgcgcttcacactcctccagctcttcatttccctaagcaatcatcaaactctcccgccattgttgttaagcccaaagccaaagaatccaacactaaacagatgaatttgttccagagagcggcggcggcagcgttggacgcggcggagggtttccttgtcagccacgagaagctacacccgcttcctaaaacggctgatcctagtgttcagatcgccggaaattttgctccggtgaatgaacagcccgtccggcgtaatcttccggtggtcggaaaacttcccgattccatcaaaggagtgtatgtgcgcaacggagctaacccacttcacgagccggtgacaggtcaccacttcttcgacggagacggtatggttcacgccgtcaaattcgaacacggttcagctagctacgcttgccggtttactcagactaaccggtttgttcaggaacgtcaattgggtcgaccggttttccccaaagccatcggtgagcttcacggccacaccggtattgcccgactcatgctattctacgccagagctgcagccggtatagtcgacccggcacacggaaccggtgtagctaacgccggtttggtctatttcaatggccggttattggctatgtcggaggatgatttaccttaccaagttcagatcactcccaatggagatttaaaaaccgttggtcggttcgattttgatggacaattagaatccacaatgattgcccacccgaaagtcgacccggaatccggtgaactcttcgctttaagctacgacgtcgtttcaaagccttacctaaaatacttccgattctcaccggacggaactaaatcaccggacgtcgagattcagcttgatcagccaacgatgatgcacgatttcgcgattacagagaacttcgtcgtcgtacctgaccagcaagtcgttttcaagctgccggagatgatccgcggtgggtctccggtggtttacgacaagaacaaggtcgcaagattcgggattttagacaaatacgccgaagattcatcgaacattaagtggattgatgctccagattgcttctgcttccatctctggaacgcttgggaagagccagaaacagatgaagtcgtcgtgatagggtcctgtatgactccaccagactcaattttcaacgagtctgacgagaatctcaagagtgtcctgtctgaaatccgcctgaatctcaaaaccggtgaatcaactcgccgtccgatcatctccaacgaagatcaacaagtcaacctcgaagcagggatggtcaacagaaacatgctcggccgtaaaaccaaattcgcttacttggctttagccgagccgtggcctaaagtctcaggattcgctaaagttgatctcactactggagaagttaagaaacatctttacggcgataaccgttacggaggagagcctctgtttctccccggagaaggaggagaggaagacgaaggatacatcctctgtttcgttcacgacgagaagacatggaaatcggagttacagatagttaacgccgttagcttagaggttgaagcaacggttaaacttccgtcaagggttccgtacggatttcacggtacattcatcggagccgatgatttggcgaagcaggtcgtgtgagttcttatgtgtaaatacgcacaaaatacatatacgtgatgaagaagcttctagaaggaaaagagagagcgagatttaccagtgggatgctctgcatatacgtccccggaatctgctcctctgtttttttttttttgctctgtttcttgtttgttgtttcttttggggtgcggtttgctagttcccttttttttggggtcaatctagaaatctgaaagattttgagggaccagcttgtagcttttgggctgtagggtagcctagccgttcgagctcagctggtttctgttattctttcacttattgttcatcgtaatgagaagtatataaaatattaaacaacaaagatatgtttgtatatgtgcatgaattaaggaacatttttttt

SEQ ID NO: 10 - protein, 599 aa, Arabidopsis thaliana
MASFTATAAVSGRWLGGNHTQPPLSSSQSSDLSYCSSLPMASRVTRKLNVSSALHTPPALHFPKQSSNSPAIVVKPKAKESNTKQMNLFQRAAAAALDAAEGFLVSHEKLHPLPKTADPSVQIAGNFAPVNEQPVRRNLPVVGKLPDSIKGVYVRNGANPLHEPVTGHHFFDGDGMVHAVKFEHGSASYACRFTQTNRFVQERQLGRPVFPKAIGELHGHTGIARLMLFYARAAAGIVDPAHGTGVANAGLVYFNGRLLAMSEDDLPYQVQITPNGDLKTVGRFDFDGQLESTMIAHPKVDPESGELFALSYDVVSKPYLKYFRFSPDGTKSPDVEIQLDQPTMMHDFAITENFVVVPDQQVVFKLPEMIRGGSPVVYDKNKVARFGILDKYAEDSSNIKWIDAPDCFCFHLWNAWEEPETDEVVVIGSCMTPPDSIFNESDENLKSVLSEIRLNLKTGESTRRPIISNEDQQVNLEAGMVNRNMLGRKTKFAYLALAEPWPKVSGFAKVDLTTGEVKKHLYGDNRYGGEPLFLPGEGGEEDEGYILCFVHDEKTWKSELQIVNAVSLEVEATVKLPSRVPYGFHGTFIGADDLAKQVV

SEQ ID NO: 11 - DNA, 950 bp, Arabidopsis thaliana
ataaaaattcacatttgcaaattttattcagtcggaatatatatttgaaacaagttttgaaatccattggacgattaaaattcattgttgagaggataaatatggatttgttcatctgaaccatgtcgttgattagtgattgactaccatgaaaaatatgttatgaaaagtataacaacttttgataaatcacatttattaacaataaatcaagacaaaatatgtcaacaataatagtagtagaagatattaattcaaattcatccgtaacaacaaaaaatcataccacaattaagtgtacagaaaaaccttttggatatatttattgtcgcttttcaatgattttcgtgaaaaggatatatttgtgtaaaataagaaggatcttgacgggtgtaaaaacatgcacaattcttaatttagaccaatcagaagacaacacgaacacttctttattataagctattaaacaaaatcttgcctattttgcttagaataatatgaagagtgactcatcagggagtggaaaatatctcaggatttgcttttagctctaacatgtcaaactatctagatgccaacaacacaaagtgcaaattcttttaatatgaaaacaacaataatatttctaatagaaaattaaaaagggaaataaaatatttttttaaaatatacaaaagaagaaggaatccatcatcaaagttttataaaattgtaatataatacaaacttgtttgcttccttgtctctccctctgtctctctcatctctcctatcttctccatatatacttcatcttcacacccaaaactccacacaaaatatctctccctctatctgcaaattttccaaagttgcatcctttcaatttccactcctctctaatataattcacattttcccactattgctgattcatttttttttgtgaattatttcaaacccacataaaa

SEQ ID NO: 12 - DNA, 1538 bp, Arabidopsis thaliana
acaaaatatctctccctctatctgcaaattttccaaagttgcatcctttcaatttccactcctctctaatataattcacattttcccactattgctgattcatttttttttgtgaattatttcaaacccacataaaaaaatctttgtttaaatttaaaaccatggatccttcatttaggttcattaaagaggagtttcctgctggattcagtgattctccatcaccaccatcttcttcttcatacctttattcatcttccatggctgaagcagccataaatgatccaacaacattgagctatccacaaccattagaaggtctccatgaatcagggccacctccatttttgacaaagacatatgacttggtggaagattcaagaaccaatcatgtcgtgtcttggagcaaatccaataacagcttcattgtctgggatccacaggccttttctgtaactctccttcccagattcttcaagcacaataacttctccagttttgtccgccagctcaacacatatggtttcagaaaggtgaatccggatcggtgggagtttgcaaacgaagggtttcttagagggcaaaagcatctcctcaagaacataaggagaagaaaaacaagtaataatagtaatcaaatgcaacaacctcaaagttctgaacaacaatctctagacaatttttgcatagaagtgggtaggtacggtctagatggagagatggacagcctaaggcgagacaagcaagtgttgatgatggagctagtgagactaagacagcaacaacaaagcaccaaaatgtatctcacattgattgaagagaagctcaagaagaccgagtcaaaacaaaaacaaatgatgagcttccttgcccgcgcaatgcagaatccagattttattcagcagctagtagagcagaaggaaaagaggaaagagatcgaagaggcgatcagcaagaagagacaaagaccgatcgatcaaggaaaaagaaatgtggaagattatggtgatgaaagtggttatgggaatgatgttgcagcctcatcctcagcattgattggtatgagtcaggaatatacatatggaaacatgtctgaattcgagatgtcggagttggacaaacttgctatgcacattcaaggacttggagataattccagtgctagggaagaagtcttgaatgtggaaaaaggaaatgatgaggaagaagtagaagatcaacaacaagggtaccataaggagaacaatgagatttatggtgaaggtttttgggaagatttgttaaatgaaggtcaaaattttgattttgaaggagatcaagaaaatgttgatgtgttaattcagcaacttggttatttgggttctagttcacacactaattaagaagaaattgaaatgatgactactttaagcatttgaatcaacttgtttcctattagtaatttggctttgtttcaatcaagtgagtcgtggactaacttattgaatttgggggttaaatccgtttcttatttttggaaataaaattgctttttgttt

SEQ ID NO: 13 - protein, 406 aa, Arabidopsis thaliana
MDPSFRFIKEEFPAGFSDSPSPPSSSSYLYSSSMAEAAINDPTTLSYPQPLEGLHESGPPPFLTKTYDLVEDSRTNHVVSWSKSNNSFIVWDPQAFSVTLLPRFFKHNNFSSFVRQLNTYGFRKVNPDRWEFANEGFLRGQKHLLKNIRRRKTSNNSNQMQQPQSSEQQSLDNFCIEVGRYGLDGEMDSLRRDKQVLMMELVRLRQQQQSTKMYLTLIEEKLKKTESKQKQMMSFLARAMQNPDFIQQLVEQKEKRKEIEEAISKKRQRPIDQGKRNVEDYGDESGYGNDVAASSSALIGMSQEYTYGNMSEFEMSELDKLAMHIQGLGDNSSAREEVLNVEKGNDEEEVEDQQQGYHKENNEIYGEGFWEDLLNEGQNFDFEGDQENVDVLIQQLGYLGSSSHTN

SEQ ID NO: 14 - DNA, 950 bp, Arabidopsis thaliana
ttttttaaaattcgttggaacttggaagggattttaaatattattttgttttccttcatttttataggttaataattgtcaaagatacaactcgatggaccaaaataaaataataaaattcgtcgaatttggtaaagcaaaacggtcgaggatagctaatatttatgcgaaacccgttgtcaaagcagatgttcagcgtcacgcacatgccgcaaaaagaatatacatcaacctcttttgaacttcacgccgttttttaggcccacaataatgctacgtcgtcttctgggttcaccctcgttttttttttaaacttctaaccgataaaataaatggtccactatttcttttcttctctgtgtattgtcgtcagagatggtttaaaagttgaaccgaactataacgattctcttaaaatctgaaaaccaaactgaccgattttcttaactgaaaaaaaaaaaaaaaaaaactgaatttaggccaacttgttgtaatatcacaaagaaaattctacaatttaattcatttaaaaataaagaaaaatttaggtaacaatttaactaagtggtctatctaaatcttgcaaattctttgactttgaccaaacacaacttaagttgacagccgtctcctctctgttgtttccgtgttattaccgaaatatcagaggaaagtccactaaaccccaaatattaaaaatagaaacattactttctttacaaaaggaatctaaattgatccctttcattcgtttcactcgtttcatatagttgtatgtatatatgcgtatgcatcaaaaagtctctttatatcctcagagtcacccaatcttatctctctctccttcgtcctcaagaaaagtaattctctgtttgtgtagttttctttaccggtgaattttctcttcgttttgtgcttcaaacgtcacccaaatcaccaagatcgatcaa

SEQ ID NO: 15 - DNA, 1720 bp, Arabidopsis thaliana
agagtcacccaatcttatctctctctccttcgtcctcaagaaaagtaattctctgtttgtgtagttttctttaccggtgaattttctcttcgttttgtgcttcaaacgtcacccaaatcaccaagatcgatcaaaatcgaaacttaacgtttcagaagatggtgcagtaccagagattaatcatccaccatggaagaaaagaagataagtttagagtttcttcagcagaggaaagtggtggaggtggttgttgctactccaagagagctaaacaaaagtttcgttgtcttctctttctctctatcctctcttgctgtttcgtcttgtctccttattacctcttcggcttctctactctctccctcctagattcgtttcgcagagaaatcgaaggtcttagctcttatgagccagttattacccctctgtgctcagaaatctccaatggaaccatttgttgtgacagaaccggtttgagatctgatatttgtgtaatgaaaggtgatgttcgaacaaactctgcttcttcctcaatcttcctcttcacctcctccaccaataacaacacaaaaccggaaaagatcaaaccttacactagaaaatgggagactagtgtgatggacaccgttcaagaactcaacctcatcaccaaagattccaacaaatcttcagatcgtgtatgcgatgtgtaccatgatgttcctgctgtgttcttctccactggtggatacaccggtaacgtataccacgagtttaacgacgggattatccctttgtttataacttcacagcattacaacaaaaaagttgtgtttgtgatcgtcgagtatcatgactggtgggagatgaagtatggagatgtcgtttcgcagctctcggattatcctctggttgatttcaatggagatacgagaacacattgtttcaaagaagcaaccgttggattacgtattcacgacgagttaactgtgaattcttctttggtcattgggaatcaaaccattgttgacttcagaaacgttttggataggggttactcgcatcgtatccaaagcttgactcaggaggaaacagaggcgaacgtgaccgcactcgatttcaagaagaagccaaaactggtgattctttcaagaaacgggtcatcaagggcgatattaaacgagaatcttctcgtggagctagcagagaaaacagggttcaatgtggaggttctaagaccacaaaagacaacggaaatggccaagatttatcgttcgttgaacacgagcgatgtaatgatcggtgtacatggagcagcaatgactcatttccttttcttgaaaccgaaaaccgttttcattcagatcatcccattagggacggactgggcggcagagacatattatggagaaccggcgaagaagctaggattgaagtacgttggttacaagattgcgccgaaagagagctctttgtatgaagaatatgggaaagatgaccctgtaatccgagatccggatagtctaaacgacaaaggatgggaatatacgaagaaaatctatctacaaggacagaacgtgaagcttgacttgagaagattcagagaaacgttaactcgttcgtatgatttctccattagaaggagatttagagaagattacttgttacatagagaagattaagaatcgtgtgatattttttttgtaaagttttgaatgacaattaaatttatttattttat

SEQ ID NO: 16 - protein, 500 aa, Arabidopsis thaliana
MVQYQRLIIHHGRKEDKFRVSSAEESGGGGCCYSKRAKQKFRCLLFLSILSCCFVLSPYYLFGFSTLSLLDSFRREIEGLSSYEPVITPLCSEISNGTICCDRTGLRSDICVMKGDVRTNSASSSIFLFTSSTNNNTKPEKIKPYTRKWETSVMDTVQELNLITKDSNKSSDRVCDVYHDVPAVFFSTGGYTGNVYHEFNDGIIPLFITSQHYNKKVVFVIVEYHDWWEMKYGDVVSQLSDYPLVDFNGDTRTHCFKEATVGLRIHDELTVNSSLVIGNQTIVDFRNVLDRGYSHRIQSLTQEETEANVTALDFKKKPKLVILSRNGSSRAILNENLLVELAEKTGFNVEVLRPQKTTEMAKIYRSLNTSDVMIGVHGAAMTHFLFLKPKTVFIQIIPLGTDWAAETYYGEPAKKLGLKYVGYKIAPKESSLYEEYGKDDPVIRDPDSLNDKGWEYTKKIYLQGQNVKLDLRRFRETLTRSYDFSIRRRFREDYLLHRED

SEQ ID NO: 17 - DNA, 950 bp, Arabidopsis thaliana
tcattacattgaaaaagaaaattaattgtctttactcatgtttattctatacaaataaaaatattaaccaaccatcgcactaacaaaatagaaatcttattctaatcacttaattgttgacaattaaatcattgaaaaatacacttaaatgtcaaatattcgttttgcatacttttcaatttaaatacatttaaagttcgacaagttgcgtttactatcatagaaaactaaatctcctaccaaagcgaaatgaaactactaaagcgacaggcaggttacataacctaacaaatctccacgtgtcaattaccaagagaaaaaaagagaagataagcggaacacgtggtagcacaaaaaagataatgtgatttaaattaaaaaacaaaaacaaagacacgtgacgacctgacgctgcaacatcccaccttacaacgtaataaccactgaacataagacacgtgtacgatcttgtctttgttttctcgatgaaaaccacgtgggtgctcaaagtccttgggtcagagtcttccatgattccacgtgtcgttaatgcaccaaacaagggtactttcggtattttggcttccgcaaattagacaaaacagctttttgtttgattgatttttctcttctctttttccatctaaattctctttgggctcttaatttctttttgagtgttcgttcgagatttgtcggagattttttcggtaaatgttgaaattttgtgggatttttttttatttctttattaaacttttttttattgaatttataaaaagggaaggtcgtcattaatcgaagaaatggaatcttccaaaatttgatattttgctgttttcttgggatttgaattgctctttatcatcaagaatctgttaaaatttctaatctaaaatctaagttgagaaaaagagagatctctaatttaaccggaattaatattctcc

SEQ ID NO: 18 - DNA, 1193 bp, Arabidopsis thaliana
aaattctctttgggctcttaatttctttttgagtgttcgttcgagatttgtcggagattttttcggtaaatgttgaaattttgtgggatttttttttatttctttattaaacttttttttattgaatttataaaaagggaaggtcgtcattaatcgaagaaatggaatcttccaaaatttgatattttgctgttttcttgggatttgaattgctctttatcatcaagaatctgttaaaatttctaatctaaaatctaagttgagaaaaagagagatctctaatttaaccggaattaatattctccgaccgaagttattatgttgcaggctcatgtcgaagaaacagagattgtctgaagaagatggagaggtagagattgagttagacttaggtctatctctaaatggaagatttggtgttgacccacttgcgaaaacaaggcttatgaggtctacgtcggttcttgatttggtggtcaacgataggtcagggctgagtaggacttgttcgttacccgtggagacggaggaagagtggaggaagaggaaggagttgcagagtttgaggaggcttgaggctaagagaaagagatcagagaagcagaggaaacataaagcttgtggtggtgaagagaaggttgtggaagaaggatctattggttcttctggtagtggttcctctggtttgtctgaagttgatactcttcttcctcctgttcaagcaacaacgaacaagtccgtggaaacaagcccttcaagtgcccaatctcagcccgagaatttgggcaaagaagcgagccaaaacattatagaggacatgccattcgtgtcaacaacaggcgatggaccgaacgggaaaaagattaatgggtttctgtatcggtaccgcaaaggtgaggaggtgaggattgtctgtgtgtgtcatggaagcttcctctcaccggcagaattcgttaagcatgctggtggtggtgacgttgcacatcccttaaagcacatcgttgtaaatccatctcccttcttgtgaccctttgggtctcttttgaggggtttgttgtatcggaaccatgttacaaatcctcattatctccgaggtgtataaacataaatttatcgaactcgcaattttcagattttgtacttaaaagaatggtttcattcgttgagattaattttagacctttttcttgtac

SEQ ID NO: 19 - protein, 231 aa, Arabidopsis thaliana
MSKKQRLSEEDGEVEIELDLGLSLNGRFGVDPLAKTRLMRSTSVLDLVVNDRSGLSRTCSLPVETEEEWRKRKELQSLRRLEAKRKRSEKQRKHKACGGEEKVVEEGSIGSSGSGSSGLSEVDTLLPPVQATTNKSVETSPSSAQSQPENLGKEASQNIIEDMPFVSTTGDGPNGKKINGFLYRYRKGEEVRIVCVCHGSFLSPAEFVKHAGGGDVAHPLKHIVVNPSPFL

SEQ ID NO: 20 - DNA, 950 bp, Arabidopsis thaliana
tttcaatgtatacaatcatcatgtgataaaaaaaaaaatgtaaccaatcaacacactgagatacggccaaaaaatggtaatacataaatgtttgtaggttttgtaatttaaatactttagttaagttatgattttattatttttgcttatcacttatacgaaatcatcaatctattggtatctcttaatcccgctttttaatttccaccgcacacgcaaatcagcaaatggttccagccacgtgcatgtgaccacatattgtggtcacagtactcgtcctttttttttcttttgtaatcaataaatttcaatcctaaaacttcacacattgagcacgtcggcaacgttagctcctaaatcataacgagcaaaaaagttcaaattagggtatatgatcaattgatcatcactacatgtctacataattaatatgtattcaaccggtcggtttgttgatactcatagttaagtatatatgtgctaattagaattaggatgaatcagttcttgcaaacaactacggtttcatataatatgggagtgttatgtacaaaatgaaagaggatggatcattctgagatgttatgggctcccagtcaatcatgttttgctcgcatatgctatcttttgagtctcttcctaaactcatagaataagcacgttggttttttccaccgtcctcctcgtgaacaaaagtacaattacattttagcaaattgaaaataaccacgtggatggaccatattatatgtgatcatattgcttgtcgtcttcgttttcttttaaatgtttacaccactacttcctgacacgtgtccctattcacatcatccttgttatatcgttttacttataaaggatcacgaacaccaaaacatcaatgtgtacgtcttttgcataagaagaaacagagagcattatcaattattaacaattacacaagacagcga

SEQ ID NO: 21 - DNA, 995 bp, Arabidopsis thaliana
aatgtgtacgtcttttgcataagaagaaacagagagcattatcaattattaacaattacacaagacagcgagattgtaaaagagtaagagagagagaatggcaggagaggcagaggctttggccacgacggcaccgttagctccggtcaccagtcagcgaaaagtacggaacgatttggaggaaacattaccaaaaccatacatggcaagagcattagcagctccagatacagagcatccgaatggaacagaaggtcacgatagcaaaggaatgagtgttatgcaacaacatgttgctttcttcgaccaaaacgacgatggaatcgtctatccttgggagacttataagggatttcgtgaccttggtttcaacccaatttcctctatcttttggaccttactcataaacttagcgttcagctacgttacacttccgagttgggtgccatcaccattattgccggtttatatcgacaacatacacaaagccaagcatgggagtgattcgagcacctatgacaccgaaggaaggtatgtcccagttaacctcgagaacatatttagcaaatacgcgctaacggttaaagataagttatcatttaaagaggtttggaatgtaaccgagggaaatcgaatggcaatcgatccttttggatggctttcaaacaaagttgaatggatactactctatattcttgctaaggacgaagatggtttcctatctaaagaagctgtgagaggttgctttgatggaagtttatttgaacaaattgccaaagagagggccaattctcgcaaacaagactaagaatgtgtgtgtttggttagcgaataaagctttttgaagaaaagcattgtgtaatttagcttctttcgtcttgttattcagtttggggatttgtataattaatgtgtttgtaaactatgtttcaaagttatataaataagagaagatgttacaaaaaaaaaaaaaagactaataagaagaatttggt

SEQ ID NO: 22 - protein, 236 aa, Arabidopsis thaliana
MAGEAEALATTAPLAPVTSQRKVRNDLEETLPKPYMARALAAPDTEHPNGTEGHDSKGMSVMQQHVAFFDQNDDGIVYPWETYKGFRDLGFNPISSIFWTLLINLAFSYVTLPSWVPSPLLPVYIDNIHKAKHGSDSSTYDTEGRYVPVNLENIFSKYALTVKDKLSFKEVWNVTEGNRMAIDPFGWLSNKVEWILLYILAKDEDGFLSKEAVRGCFDGSLFEQIAKERANSRKQD

SEQ ID NO: 23 - DNA, 950 bp, Arabidopsis thaliana
agaagaaactagaaacgttaaacgcatcaaatcaagaaattaaattgaaggtaatttttaacgccgcctttcaaatattcttcctaggagaggctacaagacgcgtatttctttcgaattctccaaaccattaccattttgatatataataccgacatgccgttgataaagtttgtatgcaaatcgttcattgggtatgagcaaatgccatccattggttcttgtaattaaatggtccaaaaatagtttgttcccactactagttactaatttgtatcactctgcaaaataatcatgatataaacgtatgtgctatttctaattaaaactcaaaagtaatcaatgtacaatgcagagatgaccataaaagaacattaaaacactacttccactaaatctatggggtgccttggcaaggcaattgaataaggagaatgcatcaagatgatatagaaaatgctattcagtttataacattaatgttttggcggaaaattttctatatattagacctttctgtaaaaaaaaaaaaatgatgtagaaaatgctattatgtttcaaaaatttcgcactagtataatacggaacattgtagtttacactgctcattaccatgaaaaccaaggcagtatataccaacattaataaactaaatcgcgatttctagcacccccattaattaattttactattatacattctctttgcttctcgaaataataaacttctctatatcattctacataataaataagaaagaaatcgacaagatctaaatttagatctattcagctttttcgcctgagaagccaaaattgtgaatagaagaaagcagtcgtcatcttcccacgtttggacgaaataaaacataacaataataaaataataaatcaaatatataaatccctaatttgtctttattactccacaattttctatgtgtatatata

SEQ ID NO: 24 - DNA, 124 bp, Arabidopsis thaliana
tgtatgtttttgttccctattatatcttctagcttctttcttcctcttcttccttaaaaattcatcctccaaaacattctatcatcaacgaaacatttcatattaaattaaataataatcgatg

SEQ ID NO: 25 - DNA, 1685 bp, Arabidopsis thaliana
gtatgtttttgttccctattatatcttctagcttctttcttcctcttcttccttaaaaattcatcctccaaaacattctatcatcaacgaaacatttcatattaaattaaataataatcgatggctgaaatttggttcttggttgtaccaatcctcatcttatgcttgcttttggtaagagtgattgtttcaaagaagaaaaagaacagtagaggtaagcttcctcctggttccatgggatggccttacttaggagagactctacaactctattcacaaaaccccaatgttttcttcacctccaagcaaaagagatatggagagatattcaaaacccgaatcctcggctatccatgcgtgatgttggctagccctgaggctgcgaggtttgtacttgtgactcatgcccatatgttcaaaccaacttatccgagaagcaaagagaagctgataggaccctctgcactctttttccaccaaggagattatcattcccatataaggaaacttgttcaatcctctttctaccctgaaaccatccgtaaactcatccctgatatcgagcacattgccctttcttccttacaatcttgggccaatatgccgattgtctccacctaccaggagatgaagaagttcgcctttgatgtgggtattctagccatatttggacatttggagagttcttacaaagagatcttgaaacataactacaatattgtggacaaaggctacaactctttccccatgagtctccccggaacatcttatcacaaagctctcatggcgagaaagcagctaaagacgatagtaagcgagattatatgcgaaagaagagagaaaagggccttgcaaacggactttcttggtcatctactcaacttcaagaacgaaaaaggtcgtgtgctaacccaagaacagattgcagacaacatcatcggagtccttttcgccgcacaggacacgacagctagttgcttaacttggattcttaagtacttacatgatgatcagaaacttctagaagctgttaaggctgagcaaaaggctatatatgaagaaaacagtagagagaagaaacctttaacatggagacaaacgaggaatatgccactgacacataaggttatagttgaaagcttgaggatggcaagcatcatatccttcacattcagagaagcagtggttgatgttgaatataagggatatttgatacctaagggatggaaagtgatgccactgtttcggaatattcatcacaatccgaaatatttttcaaaccctgaggttttcgacccatctagattcgaggtaaatccgaagccgaatacattcatgccttttggaagtggagttcatgcttgtcccgggaacgaactcgccaagttacaaattcttatatttctccaccatttagtttccaatttccgatgggaagtgaagggaggagagaaaggaatacagtacagtccatttccaatacctcaaaacggtcttcccgctacatttcgtcgacattctctttagttccttaaacctttgtagtaatctttgttgtagttagccaaatctaatccaaattcgatataaaaaatcccctttctatttttttttaaaatcattgttgtagtcttgagggggtttaacatgtaacaactatgatgaagtaaaatgtcgattccggt

SEQ ID NO: 26 - protein, 468 aa, Arabidopsis thaliana
MAEIWFLVVPILILCLLLVRVIVSKKKKNSRGKLPPGSMGWPYLGETLQLYSQNPNVFFTSKQKRYGEIFKTRILGYPCVMLASPEAARFVLVTHAHMFKPTYPRSKEKLIGPSALFFHQGDYHSHIRKLVQSSFYPETIRKLIPDIEHIALSSLQSWANMPIVSTYQEMKKFAFDVGILAIFGHLESSYKEILKHNYNIVDKGYNSFPMSLPGTSYHKALMARKQLKTIVSEIICERREKRALQTDFLGHLLNFKNEKGRVLTQEQIADNIIGVLFAAQDTTASCLTWILKYLHDDQKLLEAVKAEQKAIYEENSREKKPLTWRQTRNMPLTHKVIVESLRMASIISFTFREAVVDVEYKGYLIPKGWKVMPLFRNIHHNPKYFSNPEVFDPSRFEVNPKPNTFMPFGSGVHACPGNELAKLQILIFLHHLVSNFRWEVKGGEKGIQYSPFPIPQNGLPATFRRHSL

SEQ ID NO: 27 - DNA, 950 bp, Arabidopsis thaliana
gattctgcgaagacaggagaagccatacctttcaatctaagccgtcaacttgttcccttacgtgggatcctattatacaatccaacggttctaaatgagccacgccttccagatctaacacagtcatgctttctacagtctgcaccccttttttttttagtgttttatctacattttttcctttgtgtttaattttgtgccaacatctataacttacccctataaaaatattcaattatcacagaatacccacaatcgaaaacaaaatttaccggaataatttaattaaagctggactataatgacaattccgaaactatcaaggaataaattaaagaaactaaaaaactaaagggcattagagtaaagaagcggcaacatcagaattaaaaaactgccgaaaaaccaacctagtagccgtttatatgacaacacgtacgcaaagtctcggtaatgactcatcagttttcatgtgcaaacatattacccccatgaaataaaaaagcagagaagcgatcaaaaaaatcttcattaaaagaaccctaaatctctcatatccgccgccgtctttgcctcattttcaacaccggtgatgacgtgtaaatagatctggttttcacggttctcactactctctgtgatttttcagactattgaatcgttaggaccaaaacaagtacaaagaaactgcagaagaaaagatttgagagagatatcttacgaaacaaggtatatatttctcttgttaaatctttgaaaatactttcaaagtttcggttggattctcgaataagttaggttaaatagtcaatatagaattatagataaatcgataccttttgtttgttatcattcaatttttattgttgttacgattagtaacaacgttttagatcttgatctatatattaataatactaatactttgtttttttttgttttttttttaa

SEQ ID NO: 28 - DNA, 2828 bp, Arabidopsis thaliana
agcgatcaaaaaaatcttcattaaaagaaccctaaatctctcatatccgccgccgtctttgcctcattttcaacaccggtgatgacgtgtaaatagatctggttttcacggttctcactactctctgtgatttttcagactattgaatcgttaggaccaaaacaagtacaaagaaactgcagaagaaaagatttgagagagatatcttacgaaacaagcaaacagatgttgttgtcggcgcttggcgtcggagttggagtaggtgtgggtttaggcttggcttctggtcaagccgtcggaaaatgggccggcgggaactcgtcgtcaaataacgccgtcacggcggataagatggagaaggagatactccgtcaagttgttgacggcagagagagtaaaattactttcgatgagtttccttattatctcagtgaacaaacacgagtgcttctaacaagtgcagcttatgtccatttgaagcacttcgatgcttcaaaatatacgagaaacttgtctccagctagccgagccattctcttgtccggccctgccgagctttaccaacaaatgctagccaaagccctagctcatttcttcgatgccaagttacttcttctagacgtcaacgattttgcactcaagatacagagcaaatacggcagtggaaatacagaatcatcgtcattcaagagatctccctcagaatctgctttagagcaactatcaggactgtttagttccttctccatccttcctcagagagaagagtcaaaagctggtggtaccttgaggaggcaaagcagtggtgtggatatcaaatcaagctcaatggaaggctctagtaatcctccaaagcttcgtcgaaactcttcagcagcagctaatattagcaaccttgcatcttcctcaaatcaagtttcagcgcctttgaaacgaagtagcagttggtcattcgatgaaaagcttctcgtccaatctttatataaggtcttggcctatgtctccaaggcgaatccgattgtgttatatcttcgagacgtcgagaactttctgttccgctcacagagaacttacaacttgttccagaagcttctccagaaactcagtggaccggtcctcattctcggttcaagaattgtggacttgtcaagcgaagacgctcaagaaattgatgagaagctctctgctgttttcccttataatatcgacataagacctcctgaggatgagactcatctagtgagctggaaatcgcagcttgaacgcgacatgaacatgatccaaactcaggacaataggaaccatatcatggaagttttgtcggagaatgatcttatatgcgatgaccttgaatccatctcttttgaggacacgaaggttttaagcaattacattgaagagatcgttgtctctgctctttcctatcatctgatgaacaacaaagatcctgagtacagaaacggaaaactggtgatatcttctataagtttgtcgcatggattcagtctcttcagagaaggcaaagctggcggtcgtgagaagctgaagcaaaaaactaaggaggaatcatccaaggaagtaaaagctgaatcaatcaagccggagacaaaaacagagagtgtcaccaccgtaagcagcaaggaagaaccagagaaagaagctaaagctgagaaagttaccccaaaagctccggaagttgcaccggataacgagtttgagaaacggataagaccggaagtaatcccagcagaagaaattaacgtcacattcaaagacattggtgcacttgacgagataaaagagtcactacaagaacttgtaatgcttcctctccgtaggccagacctcttcacaggaggtctcttgaagccctgcagaggaatcttactcttcggtccaccgggtacaggtaaaacaatgctagctaaagccattgccaaagaggcaggagcgagtttcataaacgtttcgatgtcaacaataacttcgaaatggtttggagaagacgagaagaatgttagggctttgtttactctagcttcgaaggtgtcaccaaccataatatttgtggatgaagttgatagtatgttgggacagagaacaagagttggagaacatgaagctatgagaaagatcaagaatgagtttatgagtcattgggatgggttaatgactaaacctggtgaacgtatcttagtccttgctgctactaatcggcctttcgatcttgatgaagccattatcagacgattcgaacgaaggatcatggtgggactaccggctgtagagaacagagaaaagattctaagaacattgttggcgaaggagaaagtagatgaaaacttggattacaaggaactagcaatgatgacagaaggatacacaggaagtgatcttaagaatctgtgcacaaccgctgcgtataggccggtgagagaacttatacagcaagagaggatcaaagacacagagaagaagaagcagagagagcctacaaaagcaggtgaagaagatgaaggaaaagaagagagagttataacacttcgtccgttgaacagacaagactttaaagaagccaagaatcaggtggcggcgagttttgcggctgagggagcgggaatgggagagttgaagcagtggaatgaattgtatggagaaggaggatcgaggaagaaagaacaactcacttacttcttgtaatgatgatgatgaatcatgatgctggtaatggattatgaaatttggtaatgtaatagtatggtgaatttttgtttccatggttaataagagaataagaatatgatgatattgctaaaagtttgacccgt

SEQ ID NO: 29 - protein, 824 aa, Arabidopsis thaliana
MLLSALGVGVGVGVGLGLASGQAVGKWAGGNSSSNNAVTADKMEKEILRQVVDGRESKITFDEFPYYLSEQTRVLLTSAAYVHLKHFDASKYTRNLSPASRAILLSGPAELYQQMLAKALAHFFDAKLLLLDVNDFALKIQSKYGSGNTESSSFKRSPSESALEQLSGLFSSFSILPQREESKAGGTLRRQSSGVDIKSSSMEGSSNPPKLRRNSSAAANISNLASSSNQVSAPLKRSSSWSFDEKLLVQSLYKVLAYVSKANPIVLYLRDVENFLFRSQRTYNLFQKLLQKLSGPVLILGSRIVDLSSEDAQEIDEKLSAVFPYNIDIRPPEDETHLVSWKSQLERDMNMIQTQDNRNHIMEVLSENDLICDDLESISFEDTKVLSNYIEEIVVSALSYHLMNNKDPEYRNGKLVISSISLSHGFSLFREGKAGGREKLKQKTKEESSKEVKAESIKPETKTESVTTVSSKEEPEKEAKAEKVTPKAPEVAPDNEFEKRIRPEVIPAEEINVTFKDIGALDEIKESLQELVMLPLRRPDLFTGGLLKPCRGILLFGPPGTGKTMLAKAIAKEAGASFINVSMSTITSKWFGEDEKNVRALFTLASKVSPTIIFVDEVDSMLGQRTRVGEHEAMRKIKNEFMSHWDGLMTKPGERILVLAATNRPFDLDEAIIRRFERRIMVGLPAVENREKILRTLLAKEKVDENLDYKELAMMTEGYTGSDLKNLCTTAAYRPVRELIQQERIKDTEKKKQREPTKAGEEDEGKEERVITLRPLNRQDFKEAKNQVAASFAAEGAGMGELKQWNELYGEGGSRKKEQLTYFL

SEQ ID NO: 30 - DNA, 950 bp, Arabidopsis thaliana
tacttgcaaccactttgtaggaccattaactgcaaaataagaattctctaagcttcacaaggggttcgtttggtgctataaaaacattgttttaagaactggtttactggttctataaatctataaatccaaatatgaagtatggcaataataataacatgttagcacaaaaaatactcattaaattcctacccaaaaaaaatctttatatgaaactaaaacttatatacacaataatagtgatacaaagtaggtcttgatattcaactattcgggattttctggtttcgagtaattcgtataaaaggtttaagatctattatgttcactgaaatcttaactttgttttgtttccagttttaactagtagaaattgaaagttttaaaaattgttacttacaataaaatttgaatcaatatccttaatcaaaggatcttaagactagcacaattaaaacatataacgtagaatatctgaaataactcgaaaatatctgaactaagttagtagttttaaaatataatcccggtttggaccgggcagtatgtacttcaatacttgtgggttttgacgattttggatcggattgggcgggccagccagattgatctattacaaatttcacctgtcaacgctaactccgaacttaatcaaagattttgagctaaggaaaactaatcagtgatcacccaaagaaaacattcgtgaataattgtttgctttccatggcagcaaaacaaataggacccaaataggaatgtcaaaaaaaagaaagacacgaaacgaagtagtataacgtaacacacaaaaataaactagagatattaaaaacacatgtccacacatggatacaagagcatttaaggagcagaaggcacgtagtggttagaaggtatgtgatataattaatcggcccaaatagattggtaagtagtagccgtctatatca

SEQ ID NO: 31 - DNA, 104 bp, Arabidopsis thaliana
cagctcctttctactaaaacccttttactataaattctacgtacacgtaccacttcttctcctcaaattcatcaaacccatttctattccaactcccaaaaatg

SEQ ID NO: 32 - DNA, 1521 bp, Arabidopsis thaliana
agctcctttctactaaaacccttttactataaattctacgtacacgtaccacttcttctcctcaaattcatcaaacccatttctattccaactcccaaaaatggcgattcgtcttcctctgatctgtcttcttggttcattcatggtagtggcgattgcggctgatttaacaccggagcgttattggagcactgctttaccaaacactcccattcccaactctctccataatcttttgactttcgattttaccgacgagaaaagtaccaacgtccaagtaggtaaaggcggagtaaacgttaacacccataaaggtaaaaccggtagcggaaccgccgtgaacgttggaaagggaggtgtacgcgtggacacaggcaagggcaagcccggaggagggacacacgtgagcgttggcagcggaaaaggtcacggaggtggcgtcgcagtccacacgggtaaacccggtaaaagaaccgacgtaggagtcggtaaaggcggtgtgacggtgcacacgcgccacaagggaagaccgatttacgttggtgtgaaaccaggagcaaaccctttcgtgtataactatgcagcgaaggagactcagctccacgacgatcctaacgcggctctcttcttcttggagaaggacttggttcgcgggaaagaaatgaatgtccggtttaacgctgaggatggttacggaggcaaaactgcgttcttgccacgtggagaggctgaaacggtgccttttggatcggagaagttttcggagacgttgaaacgtttctcggtggaagctggttcggaagaagcggagatgatgaagaagaccattgaggagtgtgaagccagaaaagttagtggagaggagaagtattgtgcgacgtctttggagtcgatggtcgactttagtgtttcgaaacttggtaaatatcacgtcagggctgtttccactgaggtggctaagaagaacgcaccgatgcagaagtacaaaatcgcggcggctggggtaaagaagttgtctgacgataaatctgtggtgtgtcacaaacagaagtacccattcgcggtgttctactgccacaaggcgatgatgacgaccgtctacgcggttccgctcgagggagagaacgggatgcgagctaaagcagttgcggtatgccacaagaacacctcagcttggaacccaaaccacttggccttcaaagtcttaaaggtgaagccagggaccgttccggtctgccacttcctcccggagactcatgttgtgtggttcagctactagatagatctgttttctatcttattgtgggttatgtataattacgtttcagataatctatcttttgggatgttttggttatgaatatacatacatatacatatagtaatgcgtggtttccatataagagtgaaggcatctatatgtttttttttttattaacctacgtagctgtcttttgtggtctgtatcttgtggttttgcaaaaacctataataaaattagagctgaaatgttaccatttc

SEQ ID NO: 33 - protein, 392 aa, Arabidopsis thaliana
MAIRLPLICLLGSFMVVAIAADLTPERYWSTALPNTPIPNSLHNLLTFDFTDEKSTNVQVGKGGVNVNTHKGKTGSGTAVNVGKGGVRVDTGKGKPGGGTHVSVGSGKGHGGGVAVHTGKPGKRTDVGVGKGGVTVHTRHKGRPIYVGVKPGANPFVYNYAAKETQLHDDPNAALFFLEKDLVRGKEMNVRFNAEDGYGGKTAFLPRGEAETVPFGSEKFSETLKRFSVEAGSEEAEMMKKTIEECEARKVSGEEKYCATSLESMVDFSVSKLGKYHVRAVSTEVAKKNAPMQKYKIAAAGVKKLSDDKSVVCHKQKYPFAVFYCHKAMMTTVYAVPLEGENGMRAKAVAVCHKNTSAWNPNHLAFKVLKVKPGTVPVCHFLPETHVVWFSY

SEQ ID NO: 34 - DNA, 950 bp, Arabidopsis thaliana
acttattagtttaggtttccatcacctatttaattcgtaattcttatacatgcatataatagagatacatatatacaaatttatgatcatttttgcacaacatgtgatctcattcattagtatgcattatgcgaaaacctcgacgcgcaaaagacacgtaatagctaataatgttactcatttataatgattgaagcaagacgaaaacaacaacatatatatcaaattgtaaactagatatttcttaaaagtgaaaaaaaacaaagaaatataaaggacaattttgagtcagtctcttaatattaaaacatatatacataaataagcacaaacgtggttacctgtcttcatgcaatgtggactttagtttatctaatcaaaatcaaaataaaaggtgtaatagttctcgtcatttttcaaattttaaaaatcagaaccaagtgatttttgtttgagtattgatccattgtttaaacaatttaacacagtatatacgtctcttgagatgttgacatgatgataaaatacgagatcgtctcttggttttcgaattttgaactttaatagtttttttttttagggaaactttaatagttgtttatcataagattagtcacctaatggttacgttgcagtaccgaaccaattttttacccttttttctaaatgtggtcgtggcataatttccaaaagagatccaaaacccggtttgctcaactgataagccggtcggttctggtttgaaaaacaagaaataatctgaaagtgtgaaacagcaacgtgtctcggtgtttcatgagccacctgccacctcattcacgtcggtcattttgtcgtttcacggttcacgctctagacacgtgctctgtccccaccatgactttcgctgccgactcgcttcgctttgcaaactcaaacatgtgtgtatatgtaagtttcatcctaataag

SEQ ID NO: 35 - DNA, 19 bp, Arabidopsis thaliana
caaagaaaacatcaaaatg

SEQ ID NO: 36 - DNA, 700 bp, Arabidopsis thaliana
accacattaatttaaaacaaagaaaacatcaaaatggctgaaaaagtaaagtctggtcaagtttttaacctattatgcatattctcgatctttttcttcctctttgtgttatcagtgaatgtttcggctgatgtcgattctgagagagcggtgccatctgaagataaaacgacgactgtttggctaactaaaatcaaacggtccggtaaaaattattgggctaaagttagagagactttggatcgtggacagtcccacttctttcctccgaacacatattttaccggaaagaatgatgcgccgatgggagccggtgaaaatatgaaagaggcggcgacgaggagctttgagcatagcaaagcgacggtggaggaagctgctagatcagcggcagaagtggtgagtgatacggcggaagctgtgaaagaaaaggtgaagaggagcgtttccggtggagtgacgcagccgtcggagggatctgaggagctataaatacgcagttgttctaagcttatgggttttaattatttaaataattagtgtgtgtttgagatcaaaatgacacagttttgggggagtatatctccacatcatatgttgtttgcatcacatggtttctctgtatacaacgaccagatccacatcactcattctcgtccttctttttgtcatgaatacagaataatattttagattctac

SEQ ID NO: 37 - protein, 152 aa, Arabidopsis thaliana
MAEKVKSGQVFNLLCIFSIFFFLFVLSVNVSADVDSERAVPSEDKTTTVWLTKIKRSGKNYWAKVRETLDRGQSHFFPPNTYFTGKNDAPMGAGENMKEAATRSFEHSKATVEEAARSAAEVVSDTAEAVKEKVKRSVSGGVTQPSEGSEEL

SEQ ID NO: 38 - DNA, 947 bp, Arabidopsis thaliana
caaacaattactgctcaatgtatttgcgtatagagcatgtccaataccatgcctcatgatgtgagattgcgaggcggagtcagagaacgagttaaagtgacgacgttttttttgttttttttgggcatagtgtaaagtgatattaaaatttcatggttggcaggtgactgaaaataaaaatgtgtataggatgtgtttatatgctgacggaaaaatagttactcaactaatacagatctttataaagagtatataagtctatggttaatcatgaatggcaatatataagagtagatgagatttatgtttatattgaaacaagggaaagatatgtgtaattgaaacaatggcaaaatataagtcaaatcaaactggtttctgataatatatgtgttgaatcaatgtatatcttggtattcaaaaccaaaacaactacaccaatttctttaaaaaaccagttgatctaataactacattttaatactagtagctattagctgaatttcataatcaatttcttgcattaaaatttaaagtgggttttgcatttaaacttactcggtttgtattaatagactttcaaagattaaaagaaaactactgcattcagagaataaagctatcttactaaacactacttttaaagtttcttttttcacttattaatcttcttttacaaatggatctgtctctctgcatggcaaaatatcttacactaattttattttctttgtttgataacaaatttatcggctaagcatcacttaaatttaatacacgttatgaagacttaaaccacgtcacactataagaaccttacaggctgtcaaacacccttccctacccactcacatctctccacgtggcaatctttgatattgacaccttagccactacagctgtcacactcctctctcggtttcaaaacaacatctctggtataaata

SEQ ID NO: 39 - DNA, 53 bp, Arabidopsis thaliana
aatcaaaacctctcctatatctcttcaatctgatataactacccttctcaatg

SEQ ID NO: 40 - DNA, 1218 bp, Arabidopsis thaliana
aaatcaaaacctctcctatatctcttcaatctgatataactacccttctcaatggcttctaattaccgttttgccatcttcctcactctctttttcgccaccgctggtttctccgccgccgcgttggtcgaggagcagccgcttgttatgaaataccacaacggagttctgttgaaaggtaacatcacagtcaatctcgtatggtacgggaaattcacaccgatccaacggtccgtaatcgtcgatttcatccactcgctaaactccaaagacgttgcatcttccgccgcagttccttccgttgcttcgtggtggaagacgacggagaaatacaaaggtggctcttcaacactcgtcgtcgggaaacagcttctactcgagaactatcctctcggaaaatctctcaaaaatccttacctccgtgctttatccaccaaacttaacggcggtctccgttccataaccgtcgttctaacggcgaaagatgttaccgtcgaaagattctgtatgagccggtgcgggactcacggatcctccggttcgaatccccgtcgcgcagctaacggcgcggcttacgtatgggtcgggaactccgagacgcagtgccctggatattgcgcgtggccgtttcaccagccgatttacggaccacaaacgccgccgttagtagcgcctaacggtgacgttggagttgacggaatgattataaaccttgccacacttctagctaacaccgtgacgaatccgtttaataacggatattaccaaggcccaccaactgcaccgcttgaagctgtgtctgcttgtcctggtatattcgggtcaggttcttatccgggttacgcgggtcgggtacttgttgacaaaacaaccgggtctagttacaacgctcgtggactcgccggtaggaaatatctattgccggcgatgtgggatccgcagagttcgacgtgcaagactctggtttgatccaagggatgtgagtaagacacgtggcatagtagtgagagcgatgacgagatctagacggcatgtgtagtcaaaatcaagttgcacgcgagcgtgtgtataaaaaaatctttcgggtttgggtctcgggtttggattgtggatagggctctctctttgctttttgtcgttttgtaatgacgtgtaaaaactgtactcggaaatgtgaagaatgcatataaaataataaaaaatcattttgtttctact

SEQ ID NO: 41 - protein, 305 aa, Arabidopsis thaliana
MASNYRFAIFLTLFFATAGFSAAALVEEQPLVMKYHNGVLLKGNITVNLVWYGKFTPIQRSVIVDFIHSLNSKDVASSAAVPSVASWWKTTEKYKGGSSTLVVGKQLLLENYPLGKSLKNPYLRALSTKLNGGLRSITVVLTAKDVTVERFCMSRCGTHGSSGSNPRRAANGAAYVWVGNSETQCPGYCAWPFHQPIYGPQTPPLVAPNGDVGVDGMIINLATLLANTVTNPFNNGYYQGPPTAPLEAVSACPGIFGSGSYPGYAGRVLVDKTTGSSYNARGLAGRKYLLPAMWDPQSSTCKTLV

SEQ ID NO: 42 - DNA, 950 bp, Arabidopsis thaliana
atcatcgaaaggtatgtgatgcatattcccattgaaccagatttccatatattttatttgtaaagtgataatgaatcacaagatgattcaatattaaaaatgggtaactcactttgacgtgtagtacgtggaagaatagttagctatcacgcatatatatatctatgattaagtgtgtatgacataagaaactaaaatatttacctaaagtccagttactcatactgattttatgcatatatgtattatttatttatttttaataaagaagcgattggtgttttcatagaaatcatgatagattgataggtatttcagttccacaaatctagatctgtgtgctatacatgcatgtattaattttttccccttaaatcatttcagttgataatattgctctttgttccaactttagaaaaggtatgaaccaacctgacgattaacaagtaaacattaattaatctttatatatatgagataaaaccgaggatatatatgattgtgttgctgtctattgatgatgtgtcgatattatgcttgttgtaccaatgctcgagccgagcgtgatcgatgccttgacaaactatatatgtttcccgaattaattaagttttgtatcttaattagaataacatttttatacaatgtaatttctcaagcagacaagatatgtatcctatattaattactatatatgaattgccgggcacctaccaggatgtttcaaatacgagagcccattagtttccacgtaaatcacaatgacgcgacaaaatctagaatcgtgtcaaaactctatcaatacaataatatatatttcaagggcaatttcgacttctcctcaactcaatgattcaacgccatgaatctctatataaaggctacaacaccacaaaggatcatcagtcatcacaaccacattaactcttcaccactatctctcaatctct

SEQ ID NO: 43 - DNA, 837 bp, Arabidopsis thaliana
atgacagaaatgccctcgtacatgatcgagaacccaaagttcgagccaaagaaacgacgttattactcttcttcgatgcttaccatcttcttaccgatcttcacatacattatgatctttcacgttttcgaagtatcactatcttcggtctttaaagacacaaaggtcttgttcttcatctccaatactctcatcctcataatagccgccgattatggttccttctctgataaagagagtcaagacttttacggtgaatacactgtcgcagcggcaacgatgcgaaaccgagctgataactactctccgattcccgtcttgacataccgagaaaacactaaagatggagaaatcaagaaccctaaagatgtcgaattcaggaaccctgaagaagaagacgaaccgatggtgaaagatatcatttgcgtttctcctcccgagaaaatagtacgagtggtgagtgagaagaaacagagagatgatgtagctatggaagaatacaaaccagttacagaacaaactcttgctagcgaagaagcttgcaacacaagaaaccatgtgaaccctaataaaccgtacgggcgaagtaaatcagataagccacggagaaagaggctcagcgtagatacagagacgaccaaacgtaaaagttatggtcgaaagaaatcagattgctcgagatggatggttattccggagaagtgggaatatgttaaagaagaatctgaagagttttcaaagttgtccaacgaggagttgaacaaacgagtcgaagaattcatccaacggttcaatagacagatcagatcacaatcaccgcgagtttcgtctacttga

SEQ ID NO: 44 - protein, 278 aa, Arabidopsis thaliana
MTEMPSYMIENPKFEPKKRRYYSSSMLTIFLPIFTYIMIFHVFEVSLSSVFKDTKVLFFISNTLILIIAADYGSFSDKESQDFYGEYTVAAATMRNRADNYSPIPVLTYRENTKDGEIKNPKDVEFRNPEEEDEPMVKDIICVSPPEKIVRVVSEKKQRDDVAMEEYKPVTEQTLASEEACNTRNHVNPNKPYGRSKSDKPRRKRLSVDTETTKRKSYGRKKSDCSRWMVIPEKWEYVKEESEEFSKLSNEELNKRVEEFIQRFNRQIRSQSPRVSST

SEQ ID NO: 45 - DNA, 950 bp, Arabidopsis thaliana
gcgtatgctttactttttaaaatgggcctatgctataattgaatgacaaggattaaacaactaataaaagtgtagatgggttaagatgacttatttttttacttaccaatttataaatgggcttcgatgtactgaaatatatcgcgcctattaacgaggccattcaacgaatgttttaagggccctatttcgacattttaaagaacacctaggtcatcattccagaaatggatattataggatttagataatttcccacgtttggtttatttatctattttttgacgttgaccaacataatcgtgcccaaccgtttcacgcaacgaatttatatacgaaatatatatatttttcaaattaagataccacaatcaaaacagctgttgattaacaaagagattttttttttttggttttgagttacaataacgttagaggataaggtttcttgcaacgattaggaaatcgtataaaataaaatatgttataattaagtgttttattttataatgagtattaatataaataaaacctgcaaaaggatagggatattgaataataaagagaaacgaaagagcaattttacttctttataattgaaattatgtgaatgttatgtttacaatgaatgattcatcgttctatatattgaagtaaagaatgagtttattgtgcttgcataatgacgttaacttcacatatacacttattacataacatttatcacatgtgcgtctttttttttttttactttgtaaaatttcctcactttaaagacttttataacaattactagtaaaataaagttgcttggggctacaccctttctccctccaacaactctatttatagataacattatatcaaaatcaaaacatagtccctttcttctataaaggttttttcacaaccaaatttccattataaatcaaaaaataaaaacttaatta

SEQ ID NO: 46 - DNA, 1747 bp, Arabidopsis thaliana
ataaaaacttaattagtttttacagaagaaaagaaaacaatgagaggtaaatttctaagtttactgttgctcattactttggcctgcattggagtttccgccaagaagcattccacaaggcctagattaagaagaaatgatttcccacaagatttcgtttttggatctgctacttctgcttatcagtgtgaaggagctgcacatgaagatggtagaggtccaagtatctgggactccttctctgaaaaattcccagaaaagataatggatggtagtaatgggtccattgcagatgattcttacaatctttacaaggaagatgtgaatttgctgcatcaaattggcttcgatgcttaccgattttcgatctcatggtcacggattttgcctcgtgggactctaaagggaggaatcaaccaggctggaattgaatattataacaacttgattaatcaacttatatctaaaggagtgaagccatttgtcacactctttcactgggacttaccagatgcactcgaaaatgcttacggtggcctccttggagatgaatttgtgaacgatttccgagactatgcagaactttgtttccagaagtttggagatagagtgaagcagtggacgacactaaacgagccatatacaatggtacatgaaggttatataacaggtcaaaaggcacctggaagatgttccaatttctataaacctgattgcttaggtggcgatgcagccacggagccttacatcgtcggccataacctcctccttgctcatggagttgccgtaaaagtatatagagaaaagtaccaggcaactcagaaaggtgaaattggtattgccttaaacacagcatggcactacccttattcagattcatatgctgaccggttagctgcgactcgagcgactgccttcaccttcgactacttcatggagccaatcgtgtacggtagatatccaattgaaatggtcagccacgttaaagacggtcgtcttcctaccttcacaccagaagagtccgaaatgctcaaaggatcatatgatttcataggcgttaactattactcatctctttacgcaaaagacgtgccgtgtgcaactgaaaacatcaccatgaccaccgattcttgcgtcagcctcgtaggtgaacgaaatggagtgcctatcggtccagcggctggatcggattggcttttgatatatcccaagggtattcgtgatctcctactacatgcaaaattcagatacaatgatcccgtcttgtacattacagagaatggagtggatgaagcaaatattggcaaaatatttcttaacgacgatttgagaattgattactatgctcatcacctcaagatggttagcgatgctatctcgatcggggtgaatgtgaagggatatttcgcgtggtcattgatggataatttcgagtggtcggaaggatacacggtccggttcgggctagtgtttgtggactttgaagatggacgtaagaggtatctgaagaaatcagctaagtggtttaggagattgttgaagggagcgcatggtgggacgaatgagcaggtggctgttatttaataaaccacgagtcattggtcaatttagtctactgtttcttttgctctatgtacagaaagaaaataaactttccaaaataagaggtggctttgtttggactttggatgttactatatatattggtaattcttggcgtttgttagtttccaaaccaaacattaat

SEQ ID NO: 47 - protein, 514 aa, Arabidopsis thaliana
MRGKFLSLLLLITLACIGVSAKKHSTRPRLRRNDFPQDFVFGSATSAYQCEGAAHEDGRGPSIWDSFSEKFPEKIMDGSNGSIADDSYNLYKEDVNLLHQIGFDAYRFSISWSRILPRGTLKGGINQAGIEYYNNLINQLISKGVKPFVTLFHWDLPDALENAYGGLLGDEFVNDFRDYAELCFQKFGDRVKQWTTLNEPYTMVHEGYITGQKAPGRCSNFYKPDCLGGDAATEPYIVGHNLLLAHGVAVKVYREKYQATQKGEIGIALNTAWHYPYSDSYADRLAATRATAFTFDYFMEPIVYGRYPIEMVSHVKDGRLPTFTPEESEMLKGSYDFIGVNYYSSLYAKDVPCATENITMTTDSCVSLVGERNGVPIGPAAGSDWLLIYPKGIRDLLLHAKFRYNDPVLYITENGVDEANIGKIFLNDDLRIDYYAHHLKMVSDAISIGVNVKGYFAWSLMDNFEWSEGYTVRFGLVFVDFEDGRKRYLKKSAKWFRRLLKGAHGGTNEQVAVI

SEQ ID NO: 48 - DNA, 950 bp, Arabidopsis thaliana
aaagtcttatttgtgaaattttacaaatgttggaaaaaagcattttatggtgctatatttgtcaatttcccttgattatatatccttttgaaaagtaatgttttttttatgtgtgtgtattcatgaaccttggaaaaactacaaatcagatcatggtttgttttaggtgaaaaatttagaacacagttacgcaagaaagatatcggtaaatttttgtttctttgaatcgaaattaatcaaaaagtattttccattatataacaacaactaatctctgttttttttttttttttttaacaactaatctcttatcaaaatgacactacagaatcacgattgtaaatctttaaaaggcagtctgaaaaatattcatgaggatgagattttattcattcatggttgtaagtaatcattatgtaaagtttaggataaggacgttcaaaatcatataaaaaaactctacgaataaagtttatagtctatcatattgattcatatttcatagaaagttactggaaaacattacacaagtattctcgatttttacgagtttgtttagtagtcgcaaaattttattttacttttgagtatacgaacccataagctgattttctttccaagttccaataatgatatcatagtgtactcttcatgaatgtttcaagcatataattataacgttcataagtaatattctactgcatgtttgttattataaattaactaataatcgaacgtatgagttttgattgagattgttgtgctcacgaaatgaaggactcggtcaattctaaagcttaaaataagaagctcagatcttaaaactcgctttcgtcttcgtcctccatttaagtttgcgattcttttgctcttctttctctctcacatttttgtcccaaaacaataaaaagaaacaataatagaaagtgttacagaaaaagaaagaaaac

SEQ ID NO: 49 - DNA, 3048 bp, Arabidopsis thaliana
atggagagttacctcaactcgaatttcgacgttaaggcgaagcattcgtcggaggaagtgctagaaaaatggcggaatctttgcagtgtcgtcaagaacccgaaacgtcggtttcgattcactgccaatctctccaaacgttacgaagctgctgccatgcgccgcaccaaccaggagaaattaaggattgcagttctcgtgtcaaaagccgcatttcaatttatctctggtgtttctccaagtgactacaaggtgcctgaggaagttaaagcagcaggctttgacatttgtgcagacgagttaggatcaatagtggaaggtcatgatgtgaagaagctcaagttccatggtggtgttgatggtctttcaggtaagctcaaggcatgtcccaatgctggtctctcaacaggtgaacctgagcagttaagcaaacgacaagagcttttcggaatcaataagtttgcagagagtgaattacgaagtttctgggtgtttgtttgggaagcacttcaagatatgactcttatgattcttggtgtttgtgctttcgtctctttgattgttgggattgcaactgaaggatggcctcaaggatcgcatgatggtcttggcattgttgctagtattcttttagttgtgtttgtgacagcaactagtgactatagacaatctttgcagttccgggatttggataaagagaagaagaagatcacggttcaagttacgcgaaacgggtttagacaaaagatgtctatatatgatttgctccctggagatgttgttcatcttgctatcggagatcaagtccctgcagatggtcttttcctctcgggattctctgttgttatcgatgaatcgagtttaactggagagagtgagcctgtgatggtgactgcacagaaccctttccttctctctggaaccaaagttcaagatgggtcatgtaagatgttggttacaacagttgggatgagaactcaatggggaaagttaatggcaacacttagtgaaggaggagatgacgaaactccgttgcaggtgaaacttaatggagttgcaaccatcattgggaaaattggtctttccttcgctattgttacctttgcggttttggtacaaggaatgtttatgaggaagctttcattaggccctcattggtggtggtccggagatgatgcattagagcttttggagtattttgctattgctgtcacaattgttgttgttgcggttcctgaaggtttaccattagctgtcacacttagtctcgcgtttgcgatgaagaagatgatgaacgataaagcgcttgttcgccatttagcagcttgtgagacaatgggatctgcaactaccatttgtagtgacaagactggtacattaacaacaaatcacatgactgttgtgaaatcttgcatttgtatgaatgttcaagatgtagctagcaaaagttctagtttacaatctgatatccctgaagctgccttgaaactacttctccagttgatttttaataataccggtggagaagttgttgtgaacgaacgtggcaagactgagatattggggacaccaacagagactgctatattggagttaggactatctcttggaggtaagtttcaagaagagagacaatctaacaaagttattaaagttgagccttttaactcaacaaagaaaagaatgggagtagtcattgagctgcctgaaggaggacgcattcgcgctcacacgaaaggagcttcagagatagttttagcggcttgtgataaagtcatcaactcaagtggtgaagttgttccgcttgatgatgaatccatcaagttcttgaatgttacaatcgatgagtttgcaaatgaagctcttcgtactctttgccttgcttatatggatatcgaaagcgggttttcggctgatgaaggtattccggaaaaagggtttacatgcatagggattgttggtatcaaagaccctgttcgtcctggagttcgggagtccgtggaactttgtcgccgtgcgggtattatggtgagaatggttacaggagataacattaacaccgcaaaggctattgctagagaatgtggaattctcactgatgatggtatagcaattgaaggtcctgtgtttagagagaagaaccaagaagagatgcttgaactcattcccaagattcaggtcatggctcgttcttccccaatggacaagcatacactggtgaagcagttgaggactacttttgatgaagttgttgctgtgactggcgacgggacaaacgatgcaccagcgctccacgaggctgacataggattagcaatgggcattgccgggactgaagtagcgaaagagattgcggatgtcatcattctcgacgataacttcagcacaatcgtcaccgtagcgaaatggggacgttctgtttacattaacattcagaaatttgtgcagtttcaactaacagtcaatgttgttgcccttattgttaacttctcttcagcttgcttgactggaagtgctcctctaactgctgttcaactgctttgggttaacatgatcatggacacacttggagctcttgctctagctacagaacctccgaacaacgagctgatgaaacgtatgcctgttggaagaagagggaatttcattaccaatgcgatgtggagaaacatcttaggacaagctgtgtatcaatttattatcatatggattctacaggccaaagggaagtccatgtttggtcttgttggttctgactctactctcgtattgaacacacttatcttcaactgctttgtattctgccaggttttcaatgaagtaagctcgcgggagatggaagagatcgatgttttcaaaggcatactcgacaactatgttttcgtggttgttattggtgcaacagttttctttcagatcataatcattgagttcttgggcacatttgcaagcaccacacctcttacaatagttcaatggttcttcagcattttcgttggcttcttgggtatgccgatcgctgctggcttgaagaaaatacccgtgtga

SEQ ID NO: 50 - protein, 1015 aa, Arabidopsis thaliana
MESYLNSNFDVKAKHSSEEVLEKWRNLCSVVKNPKRRFRFTANLSKRYEAAAMRRTNQEKLRIAVLVSKAAFQFISGVSPSDYKVPEEVKAAGFDICADELGSIVEGHDVKKLKFHGGVDGLSGKLKACPNAGLSTGEPEQLSKRQELFGINKFAESELRSFWVFVWEALQDMTLMILGVCAFVSLIVGIATEGWPQGSHDGLGIVASILLVVFVTATSDYRQSLQFRDLDKEKKKITVQVTRNGFRQKMSIYDLLPGDVVHLAIGDQVPADGLFLSGFSVVIDESSLTGESEPVMVTAQNPFLLSGTKVQDGSCKMLVTTVGMRTQWGKLMATLSEGGDDETPLQVKLNGVATIIGKIGLSFAIVTFAVLVQGMFMRKLSLGPHWWWSGDDALELLEYFAIAVTIVVVAVPEGLPLAVTLSLAFAMKKMMNDKALVRHLAACETMGSATTICSDKTGTLTTNHMTVVKSCICMNVQDVASKSSSLQSDIPEAALKLLLQLIFNNTGGEVVVNERGKTEILGTPTETAILELGLSLGGKFQEERQSNKVIKVEPFNSTKKRMGVVIELPEGGRIRAHTKGASEIVLAACDKVINSSGEVVPLDDESIKFLNVTIDEFANEALRTLCLAYMDIESGFSADEGIPEKGFTCIGIVGIKDPVRPGVRESVELCRRAGIMVRMVTGDNINTAKAIARECGILTDDGIAIEGPVFREKNQEEMLELIPKIQVMARSSPMDKHTLVKQLRTTFDEVVAVTGDGTNDAPALHEADIGLAMGIAGTEVAKEIADVIILDDNFSTIVTVAKWGRSVYINIQKFVQFQLTVNVVALIVNFSSACLTGSAPLTAVQLLWVNMIMDTLGALALATEPPNNELMKRMPVGRRGNFITNAMWRNILGQAVYQFIIIWILQAKGKSMFGLVGSDSTLVLNTLIFNCFVFCQVFNEVSSREMEEIDVFKGILDNYVFVVVIGATVFFQIIIIEFLGTFASTTPLTIVQWFFSIFVGFLGMPIAAGLKKIPV

SEQ ID NO: 51 - DNA, 960 bp, Arabidopsis thaliana
tcaaaagtgtaatttccacaaaccaattgcgcctgcaaaagttttcaaaggatcatcaaacataatgatgaatatctcatcaccacgattttataataatgcatcttttcccaccattttttttccctcactttcttttataatcttgttcgacaacaatcatggtctaaggaaaaagttgaaaatatatattatcttagttattagaaaagaaagataatcaaatggtcaatatgcaaatggcatatgaccataaacgagtttgctagtataaagaatgatggccaacctgttaaagagagactaaaattaggtctaaaatctaggagcaatgtaaccaatacatagtatatgaaatataaaagttaatttagattttttgattagcccaaattaaagaaaaatggtatttaaaacagagactcttcatcctaaaggctaaagcaatacaatttttggttaagaaaagaaaaaaaccacaagcggaaaagaaaacaaaaaagaactatattatgatgcaacagcaacacaaagcaaaaccttgcacacacacatacaactgtaaacaagtttcttgggactctctattttctcttgctgcttgaaccaaacacaacaacgatatcccaacgagagcacaacaggtttgattatgtcggaagacaagttttgagagaaaacaaacaatattttataacaaaggagaagacttttggttagaaaaaattggtatggccattacaagacatatgggtcccaattctcatcactctctccaccaccaaaatcctcctctctctctctctcttttactctgttttcatcatctctttctctcgtctctctcaaaccctaaatacactctttctcttcttgttgtctccattctctctgtgtcatcaagcttcttttttgtgtgggttatttgaaagacactttctctgctggtatcattggagt

SEQ ID NO: 52 - DNA, 1194 bp, Arabidopsis thaliana
actctgttttcatcatctctttctctcgtctctctcaaaccctaaatacactctttctcttcttgttgtctccattctctctgtgtcatcaagcttcttttttgtgtgggttatttgaaagacactttctctgctggtatcattggagtctagggttttgttattgacatgcgtggtgtgtcagaattggaggtggggaagagtaatcttccggcggagagtgagctggaattgggattagggctcagcctcggtggtggcgcgtggaaagagcgtgggaggattcttactgctaaggattttccttccgttgggtctaaacgctctgctgaatcttcctctcaccaaggagcttctcctcctcgttcaagtcaagtggtaggatggccaccaattgggttacacaggatgaacagtttggttaataaccaagctatgaaggcagcaagagcggaagaaggagacggggagaagaaagttgtgaagaatgatgagctcaaagatgtgtcaatgaaggtgaatccgaaagttcagggcttagggtttgttaaggtgaatatggatggagttggtataggcagaaaagtggatatgagagctcattcgtcttacgaaaacttggctcagacgcttgaggaaatgttctttggaatgacaggtactacttgtcgagaaaaggttaaacctttaaggcttttagatggatcatcagactttgtactcacttatgaagataaggaaggggattggatgcttgttggagatgttccatggagaatgtttatcaactcggtgaaaaggcttcggatcatgggaacctcagaagctagtggactagctccaagacgtcaagagcagaaggatagacaaagaaacaaccctgtttagcttcccttccaaagctggcattgtttatgtattgtttgaggtttgcaatttactcgatactttttgaagaaagtattttggagaatatggataaaagcatgcagaagcttagatatgatttgaatccggttttcggatatggttttgcttaggtcattcaattcgtagttttccagtttgtttcttctttggctgtgtaccaattatctatgttctgtgagagaaagctcttgtttatttgttctctcagattgtaaatagttgaagttatctaattaatgtgataagagttatgtttatgattcc

SEQ ID NO: 53 - protein, 239 aa, Arabidopsis thaliana
MRGVSELEVGKSNLPAESELELGLGLSLGGGAWKERGRILTAKDFPSVGSKRSAESSSHQGASPPRSSQVVGWPPIGLHRMNSLVNNQAMKAARAEEGDGEKKVVKNDELKDVSMKVNPKVQGLGFVKVNMDGVGIGRKVDMRAHSSYENLAQTLEEMFFGMTGTTCREKVKPLRLLDGSSDFVLTYEDKEGDWMLVGDVPWRMFINSVKRLRIMGTSEASGLAPRRQEQKDRQRNNPV

SEQ ID NO: 54 - DNA, 950 bp, Arabidopsis thaliana
gacgggtcatcacagattcttcgtttttttatagatagaaaaggaataacgttaaaagtatacaaattatatgcaagagtcattcgaaagaattaaataaagagatgaactcaaaagtgattttaaattttaatgataagaatatacatctcacagaaatcttttatttgacatgtaaaatcttgttttcacctatcttttgttagtaaacaagaatatttaatttgagcctcacttggaacgtgataataatatacatcttatcataattgcatattttgcggatagtttttgcatggggagattaaaggcttaataaagccttgaatttccgaggggaggaatcatgttttatacttgcaaactatacaaccatctgcatcgataattggtgttaatacatgcaaggattatacactaaaacaaatcatttatttccttacaaaaagagagtcgactgtgagtcacattctgtgacaaggaaaggtcaagaaccatcgcttttatcatcattctctttgctaacaacttacaaccacacaaacgcaagagttccattctcatggagaagaacatattatgcaaaataatgtatgtcgatcgatagagaaaaggatccacaattattgctccatctcaaaagcttctttagtacacgatacatgtatcatgtaaatagaaatatgaaagatacaatacacgacccattctcataaagatagcaacatttcatgttatgtaaagagtcttccttaggacacatgcattaaaactaaggattaccaacccacttactcctcactccaaccaaatatcaatcatctattttgggtccttcactcataagtcaactctcatgccttcctctataaataccgtaccctacgcatcccttagttctacatcacataaaaacaatcatagcaaaaacatatatcctcaaattaatt

SEQ ID NO: 55 - DNA, 918 bp, Arabidopsis thaliana
atggatcatgaggaaattccatccacgccctcaacgccggcgacaaccccggggactccaggagcgccgctctttggaggattcgaagggaagaggaatggacacaatggtagatacacaccaaagtcacttctcaaaagctgcaaatgtttcagtgttgacaatgaatgggctcttgaagatggaagactccctccggtcacttgctctctccctccccctaacgtttccctctaccgcaagttgggagcagagtttgttgggacattgatcctgatattcgccggaacagcgacggcgatcgtgaaccagaagacagatggagctgagacgcttattggttgcgccgcctcggctggtttggcggttatgatcgttatattatcgaccggtcacatctccggggcacatctcaatccggctgtaaccattgcctttgctgctctcaaacacttcccttggaaacacgtgccggtgtatatcggagctcaggtgatggcctccgtgagtgcggcgtttgcactgaaagcagtgtttgaaccaacgatgagcggtggcgtgacggtgccgacggtgggtctcagccaagctttcgccttggaattcattatcagcttcaacctcatgttcgttgtcacagccgtagccaccgacacgagagctgtgggagagttggcgggaattgccgtaggagcaacggtcatgcttaacatacttatagctggacctgcaacttctgcttcgatgaaccctgtaagaacactgggtccagccattgcagcaaacaattacagagctatttgggtttacctcactgcccccattcttggagcgttaatcggagcaggtacatacacaattgtcaagttgccagaggaagatgaagcacccaaagagaggaggagcttcagaagatga

SEQ ID NO: 56 - protein, 305 aa, Arabidopsis thaliana
MDHEEIPSTPSTPATTPGTPGAPLFGGFEGKRNGHNGRYTPKSLLKSCKCFSVDNEWALEDGRLPPVTCSLPPPNVSLYRKLGAEFVGTLILIFAGTATAIVNQKTDGAETLIGCAASAGLAVMIVILSTGHISGAHLNPAVTIAFAALKHFPWKHVPVYIGAQVMASVSAAFALKAVFEPTMSGGVTVPTVGLSQAFALEFIISFNLMFVVTAVATDTRAVGELAGIAVGATVMLNILIAGPATSASMNPVRTLGPAIAANNYRAIWVYLTAPILGALIGAGTYTIVKLPEEDEAPKERRSFRR

SEQ ID NO: 57 - DNA, 950 bp, Arabidopsis thaliana
cgctccagaccactgtttgctttcctctgattaaccaatctcaattaaactactaatttataattcaagataattagataaccaatcttaaaatttggaatcttcttccctcacttgatattacaaaaaaaaaactgatttatcatacggttaattcaagaaaacagcaaaaaaattgcactataatgcaaaacatcaattaattacattcgattaaaaaatcatcattgaatctaaaatggcctcaaatctattgagcatttgtcatgtgcctaaaatggttcaggagttttacatctaatcacataaaaagcaaacaataaccaaaaaaattgcattttagcaaatcaaatacttatatatatacgtatgattaagcgtcatgactttaaaacctctgtaaaattttgatttatttttcgatgcttttattttttaaccaatagtaataaagtccaaatcttaaatacgaaaaaatgtttctttctaagcgaccaacaaaatggtccaaatcacagaaaatgttccataatccaggcccattaagctaatcaccaagtaatacattacacgtcaccaattaatacattacacgtacggccttctctcttcacgagtaatatgcaaacaaacgtacattagctgtaatgtactcactcatgcaacgtcttaacctgccacgtattacgtaattacaccactccttgttcctaacctacgcatttcactttagcgcatgttagtcaaaaaacacaaacataaactacaaataaaaaaactcaaaacaaaacccaatgaacgaacggaccagccccgtctcgattgatggaacagtgacaacagtcccgttttctcgggcataacggaaacggtaaccgtctctctgtttcatttgcaacaacaccatttttataaataaaaacacatttaaataaaaaattattaaaacc

SEQ ID NO: 58 - DNA, 153 bp, Arabidopsis thaliana
tatatccaaacaaatgaatgtgttaaaccttcactcttctctccacacaaaattcaaaaacctcacatttcacttctctcttctcgcttcttctagatctcaccggtttatctagctccggtttgattcatctccggttatggggagagaatg

SEQ ID NO: 59 - DNA, 2017 bp, Arabidopsis thaliana
atatatccaaacaaatgaatgtgttaaaccttcactcttctctccacacaaaattcaaaaacctcacatttcacttctctcttctcgcttcttctagatctcaccggtttatctagctccggtttgattcatctccggttatggggagagaatgaggagttaccgttttagtgattatctacacatgtctgtttcattctctaacgatatggatttgttttgtggagaagactccggtgtgttttccggtgagtcaacggttgatttctcgtcttccgaggttgattcatggcctggtgattctatcgcttgttttatcgaagacgagcgtcacttcgttcctggacatgattatctctctagatttcaaactcgatctctcgatgcttccgctagagaagattccgtcgcatggattctcaaggtacaagcgtattataactttcagcctttaacggcgtacctcgccgttaactatatggatcggtttctttacgctcgtcgattaccggaaacgagtggttggccaatgcaacttttagcagtggcatgcttgtctttagctgcaaagatggaggaaattctcgttccttctctttttgattttcaggttgcaggagtgaagtatttatttgaagcaaaaactataaaaagaatggaacttcttgttctaagtgtgttagattggagactaagatcggttacaccgtttgatttcattagcttctttgcttacaagatcgatccttcgggtacctttctcgggttctttatctcccatgctacagagattatactctccaacataaaagaagcgagctttcttgagtactggccatcgagtatagctgcagccgcgattctctgtgtagcgaacgagttaccttctctatcctctgttgtcaatccccacgagagccctgagacttggtgtgacggattgagcaaagagaagatagtgagatgctatagactgatgaaagcgatggccatcgagaataaccggttaaatacaccaaaagtgatagcaaagcttcgagtgagtgtaagggcatcatcgacgttaacaaggccaagtgatgaatcctctttctcatcctcttctccttgtaaaaggagaaaattaagtggctattcatgggtaggtgatgaaacatctacctctaattaaaatttggggagtgaaagtagaggaccaaggaaacaaaacctagaagaaaaaaaaccctcttctgtttaagtagagtatattttttaacaagtacatagtaataagggagtgatgaagaaaagtaaaagtgtttattggctgagttaaagtaattaagagttttccaaccaaggggaaggaataagagttttggttacaatttcttttatggaaagggtaaaaattgggttttggggttggttggttggttgggagagacgaagctcatcattaatggctttgcagattcccaagaaagcaaaatgagtaagtgagtgtaacacacacgtgttagagaaaagatatgatcatgtgagtgtgtgtgtgtgagagagagagagaagagtatttgcattagagtcctcatcacacaggtactgatggataagacaggggagcgtttgcaaaagatttgtgagtggagatttttctgagctctttgtcttaatggatcgcagcagttcatgggacccttcctcagcttcatcatcaaacaaaaaaaaaatcaagttgcgaagtatatataatttgtttttttgtttggatttttaagatttttgattccttgtgtgtgacttcacgtgacggaggcgtgtgtctcacgtgtttgttttctcttcaaatcttttattttggcgggaaattttgtgtttttgatttctacgtattcgtggactccaaatgagttttgtcacggtgcgttttagtagcgtttgcatgcgtgtaaggtgtcacgtatgtgtatatatatgatttttttttggtttcttgaaaggttgaattttataaataaaacgtttctattat

SEQ ID NO: 60 - protein, 339 aa, Arabidopsis thaliana
MRSYRFSDYLHMSVSFSNDMDLFCGEDSGVFSGESTVDFSSSEVDSWPGDSIACFIEDERHFVPGHDYLSRFQTRSLDASAREDSVAWILKVQAYYNFQPLTAYLAVNYMDRFLYARRLPETSGWPMQLLAVACLSLAAKMEEILVPSLFDFQVAGVKYLFEAKTIKRMELLVLSVLDWRLRSVTPFDFISFFAYKIDPSGTFLGFFISHATEIILSNIKEASFLEYWPSSIAAAAILCVANELPSLSSVVNPHESPETWCDGLSKEKIVRCYRLMKAMAIENNRLNTPKVIAKLRVSVRASSTLTRPSDESSFSSSSPCKRRKLSGYSWVGDETSTSN

SEQ ID NO: 61 - DNA, 950 bp, Arabidopsis thaliana
tttaaacataacaatgaattgcttggatttcaaactttattaaatttggattttaaattttaatttgattgaattatacccccttaattggataaattcaaatatgtcaactttttttttttgtaagatttttttatggaaaaaaaaattgattattcactaaaaagatgacaggttacttataatttaatatatgtaaaccctaaaaagaagaaaatagtttctgttttcactttaggtcttattatctaaacttctttaagaaaatcgcaataaattggtttgagttctaactttaaacacattaatatttgtgtgctatttaaaaaataatttacaaaaaaaaaaacaaattgacagaaaatatcaggttttgtaataagatatttcctgataaatatttagggaatataacatatcaaaagattcaaattctgaaaatcaagaatggtagacatgtgaaagttgtcatcaatatggtccacttttctttgctctataacccaaaattgaccctgacagtcaacttgtacacgcggccaaacctttttataatcatgctatttatttccttcatttttattctatttgctatctaactgatttttcattaacatgataccagaaatgaatttagatggattaattcttttccatccacgacatctggaaacacttatctcctaattaaccttactttttttttagtttgtgtgctccttcataaaatctatattgtttaaaacaaaggtcaataaatataaatatggataagtataataaatctttattggatatttctttttttaaaaaagaaataaatcttttttggatattttcgtggcagcatcataatgagagactacgtcgaaactgctggcaaccacttttgccgcgtttaatttctttctgaggcttatataaatagatcaaaggggaaagtgagatat

SEQ ID NO: 62 - DNA, 703 bp, Arabidopsis thaliana
aaagaaaatgggtttgagaagaacatggttggttttgtacattctcttcatctttcatcttcagcacaatcttccttccgtgagctcacgaccttcctcagtcgatacaaaccacgagactctcccttttagtgtttcaaagccagacgttgttgtgtttgaaggaaaggctcgggaattagctgtcgttatcaaaaaaggaggaggtggaggaggtggaggacgcggaggcggtggagcacgaagcggcggtaggagcaggggaggaggaggtggcagcagtagtagccgcagccgtgactggaaacgcggcggaggggtggttccgattcatacgggtggtggtaatggcagtctgggtggtggatcggcaggatcacatagatcaagcggcagcatgaatcttcgaggaacaatgtgtgcggtctgttggttggctttatcggttttagccggtttagtcttggttcagtagggttcagagtaattattggccatttatttattggttttgtaacgtttatgtttgtggtccggtctgatatttatttgggcaaacggtacattaaggtgtagactgttaatattatatgtagaaagagattcttagcaggattctactggtagtattaagagtgagttatctttagtatgccatttgtaaatggaaatttaatgaaataagaaattgtgaaatttaaac

SEQ ID NO: 63 - protein, 157 aa, Arabidopsis thaliana
KKMGLRRTWLVLYILFIFHLQHNLPSVSSRPSSVDTNHETLPFSVSKPDVVVFEGKARELAVVIKKGGGGGGGGRGGGGARSGGRSRGGGGGSSSSRSRDWKRGGGVVPIHTGGGNGSLGGGSAGSHRSSGSMNLRGTMCAVCWLALSVLAGLVLVQ

(19) United States
(12) Patent Application Publication
(10) Pub. No.: US 2010/0037346 A1
(43) Pub. Date: Feb. 11, 2010

(54) PROMOTER, PROMOTER CONTROL ELEMENTS, AND COMBINATIONS, AND USES THEREOF

(75) Inventors: Zhihong COOK, Aliso Viejo, CA (US); Yiwen Fang, Los Angeles, CA (US); Kenneth A. Feldmann, Newbury Park, CA (US); Edward A. Kiegle, Chester, VT (US); Shing Kwok, Woodland Hills, CA (US); Roger Pennell, Malibu, CA (US); Richard Schneeberger, Carlsbad, CA (US); Chuan-Yin Wu, Newbury Park, CA (US); Nestor Apuya, Culver City, CA (US); Diane K. Jofuku, Arlington, CA (US); Jonathan Donson, Oak Park, CA (US); Leonard Medrano, Tucson, AZ (US)

Correspondence Address: BIRCH STEWART KOLASCH & BIRCH, PO BOX 747, FALLS CHURCH, VA, 22040-0747, US

(73) Assignee: CERES, INC., Thousand Oaks, CA (US)

(21) Appl. No.: 12/575,402

(22) Filed: Oct. 7, 2009

Related U.S. Application Data
(63) Continuation of application No. 11/097,589, filed on Apr. 1, 2005, now abandoned.
(60) Provisional application No. 60/558,869, filed on Apr. 1, 2004.

(51) Int. Cl. C12N 15/82; C12N 15/11; C12N 15/00; C12N 5/10; C12N 1/19; C12N 1/21; C12N 5/04; A01H 5/00

(52) U.S. Cl. 800/278; 536/24.1; 435/320.1; 435/325; 435/254.2; 435/252.3; 435/419; 800/298

(57) ABSTRACT

The present invention is directed to promoter sequences and promoter control elements, polynucleotide constructs comprising the promoters and control elements, and methods of identifying the promoters, control elements, or fragments thereof. The invention further relates to the use of the present promoters or promoter control elements to modulate transcript levels.

### CROSS REFERENCE TO RELATED APPLICATION

This application is a Continuation of co-pending application Ser. No. 11/097,589, filed on Apr. 1, 2005, the entire contents of which are hereby incorporated by reference and for which priority is claimed under 35 U.S.C. §120.

The Nonprovisional application Ser. No. 11/097,589, filed on Apr. 1, 2005, claims priority under 35 U.S.C. §119(e) on U.S. Provisional Application No. 60/558,869 filed on Apr. 1, 2004, the entire contents of which are hereby incorporated by reference.

### FIELD OF THE INVENTION

The present invention relates to promoters and promoter control elements that are useful for modulating transcription of a desired polynucleotide. Such promoters and promoter control elements can be included in polynucleotide constructs, expression cassettes, vectors, or inserted into the chromosome or as an exogenous element, to modulate in vivo and in vitro transcription of a polynucleotide. Host cells, including plant cells, and organisms, such as regenerated plants therefrom, with desired traits or characteristics using polynucleotides comprising the promoters and promoter control elements of the present invention are also a part of the invention.

### BACKGROUND OF THE INVENTION

This invention relates to the field of biotechnology and, in particular, to specific promoter sequences and promoter control element sequences which are useful for the transcription of polynucleotides in a host cell or transformed host organism.

One of the primary goals of biotechnology is to obtain organisms, such as plants, mammals, yeast, and prokaryotes having particular desired characteristics or traits. Examples of these characteristic or traits abound and may include, for example, in plants, virus resistance, insect resistance, herbicide resistance, enhanced stability or additional nutritional value. Recent advances in genetic engineering have enabled researchers in the field to incorporate polynucleotide sequences into host cells to obtain the desired qualities in the organism of choice. This technology permits one or more polynucleotides from a source different than the organism of choice to be transcribed by the organism of choice. If desired, the transcription and/or translation of these new polynucleotides can be modulated in the organism to exhibit a desired characteristic or trait. Alternatively, new patterns of transcription and/or translation of polynucleotides endogenous to the organism can be produced. Both approaches can be used at the same time.

### SUMMARY OF THE INVENTION

The present invention is directed to isolated polynucleotide sequences that comprise promoters and promoter control elements from plants, especially Arabidopsis thaliana, Glycine max, Oryza sativa, and Zea mays, and other promoters and promoter control elements functional in plants.

It is an object of the present invention to provide isolated polynucleotides that are promoter sequences. These promoter sequences comprise, for example,
(1) a polynucleotide having a nucleotide sequence as set forth in Table 1, in the section entitled “The predicted promoter sequence” or fragment thereof;
(2) a polynucleotide having a nucleotide sequence having at least 80% sequence identity to a sequence as set forth in Table 1, in the section entitled “The predicted promoter sequence” or fragment thereof; and
(3) a polynucleotide having a nucleotide sequence which hybridizes to a sequence as set forth in Table 1, in the section entitled “The predicted promoter sequence” under a condition establishing a Tm-20° C.

It is another object of the present invention to provide isolated polynucleotides that are promoter control element sequences. These promoter control element sequences comprise, for example,
(1) a polynucleotide having a nucleotide sequence as set forth in Table 1, in the section entitled “The predicted promoter sequence” or fragment thereof;
(2) a polynucleotide having a nucleotide sequence having at least 80% sequence identity to a sequence as set forth in Table 1, in the section entitled “The predicted promoter sequence” or fragment thereof; and
(3) a polynucleotide having a nucleotide sequence which hybridizes to a sequence as set forth in Table 1, in the section entitled “The predicted promoter sequence” under a condition establishing a Tm-20° C.

Promoter or promoter control element sequences of the present invention are capable of modulating preferential transcription.

In another embodiment, the present promoter control elements are capable of serving as or fulfilling the function, for example, as a core promoter, a TATA box, a polymerase binding site, an initiator site, a transcription binding site, an enhancer, an inverted repeat, a locus control region, or a scaffold/matrix attachment region.

It is yet another object of the present invention to provide a polynucleotide that includes at least a first and a second promoter control element. The first promoter control element is a promoter control element sequence as discussed above, and the second promoter control element is heterologous to the first control element. Moreover, the first and second control elements are operably linked. Such promoters may modulate transcript levels preferentially in a tissue or under particular conditions.

In another embodiment, the present isolated polynucleotide comprises a promoter or a promoter control element as described above, wherein the promoter or promoter control element is operably linked to a polynucleotide to be transcribed.

In another embodiment of the present vector, the promoter and promoter control elements of the instant invention are operably linked to a heterologous polynucleotide that is a regulatory sequence.

It is another object of the present invention to provide a host cell comprising an isolated polynucleotide or vector as described above or fragment thereof. Host cells include, for instance, bacterial, yeast, insect, mammalian, and plant. The host cell can comprise a promoter or promoter control element exogenous to the genome. Such a promoter can modulate transcription in cis- and in trans-.

In yet another embodiment, the present host cell is a plant cell capable of regenerating into a plant.

It is yet another embodiment of the present invention to provide a plant comprising an isolated polynucleotide or vector described above.

It is another object of the present invention to provide a method of modulating transcription in a sample that contains either a cell-free system of transcription or host cell. This method comprises providing a polynucleotide or vector according to the present invention as described above, and contacting the sample of the polynucleotide or vector with conditions that permit transcription.

In another embodiment of the present method, the polynucleotide or vector preferentially modulates
(a) constitutive transcription,
(b) stress induced transcription,
(c) light induced transcription,
(d) dark induced transcription,
(e) leaf transcription,
(f) root transcription,
(g) stem or shoot transcription,
(h) silique transcription,
(i) callus transcription,
(j) flower transcription,
(k) immature bud and inflorescence specific transcription, or
(l) senescing induced transcription
(m) germination transcription.
   Other and further objects of the present invention will be made clear or become apparent from the following description.

### BRIEF DESCRIPTION OF THE TABLES AND FIGURES

### Table 1

Table 1 consists of the Expression Reports for each promoter of the invention providing the nucleotide sequence for each promoter and details for expression driven by each of the nucleic acid promoter sequences as observed in transgenic plants. The results are presented as summaries of the spatial expression, which provides information as to gross and/or specific expression in various plant organs and tissues. The observed expression pattern is also presented, which gives details of expression during different generations or different developmental stages within a generation. Additional information is provided regarding the associated gene, the GenBank reference, the source organism of the promoter, and the vector and marker genes used for the construct. The following symbols are used consistently throughout the Table:
T1: First generation transformant
T2: Second generation transformant
T3: Third generation transformant
(L): low expression level
(M): medium expression level
(H): high expression level

Each row of the table begins with heading of the data to be found in the section. The following provides a description of the data to be found in each section:

| Heading in Table 1 | Description |
|---|---|
| Promoter | Identifies the particular promoter by its construct |
| | ID. |
| Modulates the gene: | This row states the name of the gene modulated by |
| | the promoter |
| The GenBank description of the gene: | This field gives the Locus Number of the gene as |
| | well as the accession number. |
| The promoter sequence: | Identifies the nucleic acid promoter sequence in |
| | question. |
| The promoter was cloned from the organism: | Identifies the source of the DNA template used to |
| | clone the promoter. |
| Alternative nucleotides: | Identifies alternative nucleotides in the promoter |
| | sequence at the base pair positions identified in the |
| | column called “Sequence (bp)” based upon |
| | nucleotide difference between the two species of |
| | Arabidopsis. |
| The promoter was cloned in the vector: | Identifies the vector used into which a promoter was |
| | cloned. |
| When cloned into the vector the promoter was | Identifies the type of marker linked to the promoter. |
| operably linked to a marker, which was the type: | The marker is used to determine patterns of gene |
| | expression in plant tissue. |
| Promoter-marker vector was tested in: | Identifies the organism in which the promoter- |
| | marker vector was tested. |
| Generation screened: T1 Mature T2 | Identifies the plant generation(s) used in the |
| Seedling T2 Mature T3 Seedling | screening process. T1 plants are those plants |
| | subjected to the transformation event while the T2 |
| | generation plants are from the seeds collected from |
| | the T1 plants and T3 plants are from the seeds of T2 |
| | plants. |
| The spatial expression of the promoter-marker | Identifies the specific parts of the plant where |
| vector was found observed in and would be useful in | various levels of GFP expression are observed. |
| expression in any or all of the following: | Expression levels are noted as either low (L), |
| | medium (M), or high (H). |
| Observed expression pattern of the promoter-marker | Identifies a general explanation of where GFP |
| vector was in: | expression in different generations of plants was |
| T1 mature: | observed. |
| T2 seedling: | |
| The promoter can be of use in the following trait | Identifies which traits and subtraits the promoter |
| and sub-trait areas: (search for the trait and sub-trait | cDNA can modulate |
| table) | |
| The promoter has utility in: | Identifies a specific function or functions that can be |
| | modulated using the promoter cDNA. |
| Misc. promoter information: | “Bidirectionality” is determined by the number of |
| Bidirectionality: | base pairs between the promoter and the start codon |
| Exons: | of a neighboring gene. A promoter is considered |
| Repeats: | bidirectional if it is closer than 200 bp to a start |
| | codon of a gene 5′ or 3′ to the promoter. “Exons” |
| | (or any coding sequence) identifies if the promoter |
| | has overlapped with either the modulating gene's or |
| | other neighboring gene's coding sequence. A “fail” |
| | for exons means that this overlap has occurred. |
| | “Repeats” identifies the presence of normally |
| | occurring sequence repeats that randomly exist |
| | throughout the genome. A “pass” for repeats |
| | indicates a lack of repeats in the promoter. |
| Optional Promoter Fragments: An overlap with | Identifies the specific nucleotides overlapping the |
| the UTR/exon region of the endogenous coding | UTR region or exon of a neighboring gene. The |
| sequence to the promoter occurs at base pairs . | orientation relative to the promoter is designated |
| | with a 5′ or 3′. |
| The Ceres cDNA ID of the endogenous coding | Identifies the number associated with the Ceres |
| sequence to the promoter: | cDNA that corresponds to the endogenous cDNA |
| | sequence of the promoter. |
| cDNA nucleotide sequence: | The nucleic acid sequence of the Ceres cDNA |
| | matching the endogenous cDNA region of the |
| | promoter. |
| Coding sequence: | A translated protein sequence of the gene modulated |
| | by a protein encoded by a cDNA |
| Microarray Data: Microarray Data shows that the | Microarray data is identified along with the |
| coding sequence was expressed in the following | corresponding experiments along with the |
| experiments, which shows that the promoter would | corresponding gene expression. Gene expression is |
| useful to modulate expression in situations similar to | identified by a “+” or a “−” in the |
| the following: | “SIGN(LOG_RATIO)” column. A “+” notation |
| | indicates the cDNA is upregulated while a “−” |
| | indicates that the cDNA is downregulated. The |
| | “SHORT_NAME” field describes the experimental |
| | conditions. |
| Microarray Experiment Parameters: The parameters | Parameters for microarray experiments include age, |
| for the microarray experiments listed above by | organism, specific tissues, age, treatments and other |
| EXPT_REP_ID and Short_Name are as follow | distinguishing characteristics or features. |
| below: | |

The section of Table 1 entitled “optional promoter fragments” identifies the co-ordinates of nucleotides of the promoter that represent optional promoter fragments. The optional promoter fragments comprise the 5′ UTR and any exon(s) of the endogenous coding region. The optional promoter fragments may also comprise any exon(s) and the 3′ or 5′ UTR of the gene residing upstream of the promoter (that is, 5′ to the promoter). The optional promoter fragments also include any intervening sequences that are introns or sequence occurring between exons or an exon and the UTR.

The information on optional promoter fragments can be used to generate either reduced promoter sequences or “core” promoters. A reduced promoter sequence is generated when at least one optional promoter fragment is deleted. Deletion of all optional promoter fragments generates a “core” promoter.

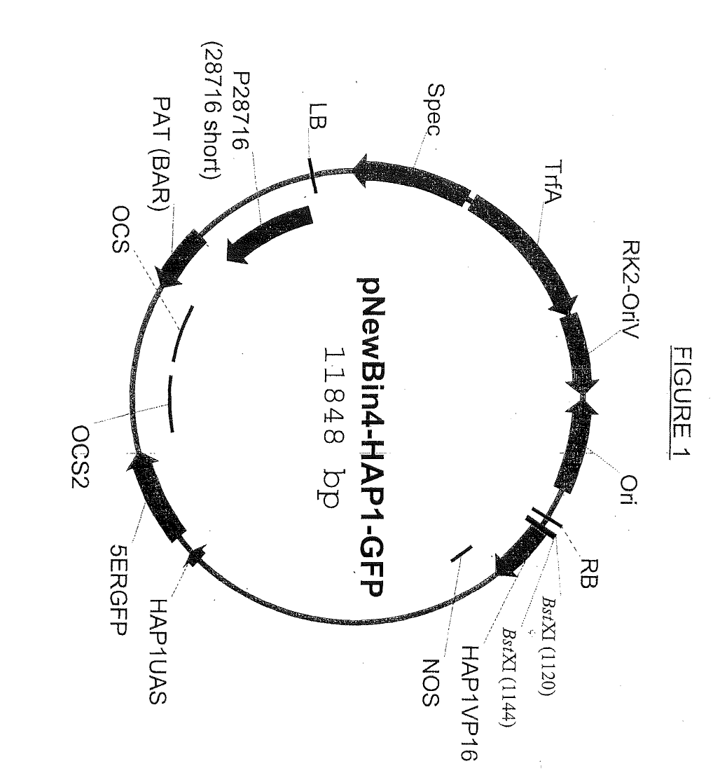

FIG. 1

FIG. 1 is a schematic representation of the vector pNewBin4-HAP1-GFP. The definitions of the abbreviations used in the vector map are as follows:
Ori—the origin of replication used by an E. coli host
RB—sequence for the right border of the T-DNA from pMOG800
BstXI—restriction enzyme cleavage site used for cloning
HAP1VP16—coding sequence for a fusion protein of the HAP1 and VP16 activation domains
NOS—terminator region from the nopaline synthase gene
HAP1UAS—the upstream activating sequence for HAP1
5ERGFP—the green fluorescent protein gene that has been optimized for localization to the endoplasmic reticulum
OCS2—the terminator sequence from the octopine synthase 2 gene
OCS—the terminator sequence from the octopine synthase gene
p28716 (a.k.a 28716 short)—promoter used to drive expression of the PAT (BAR) gene
PAT (BAR)—a marker gene conferring herbicide resistance
LB—sequence for the left border of the T-DNA from pMOG800
Spec—a marker gene conferring spectinomycin resistance
TrfA—transcription repression factor gene
RK2-OriV—origin of replication for Agrobacterium

### DETAILED DESCRIPTION OF THE INVENTION

### 1. Definitions

Chimeric: The term “chimeric” is used to describe polynucleotides or genes, as defined supra, or constructs wherein at least two of the elements of the polynucleotide or gene or construct, such as the promoter and the polynucleotide to be transcribed and/or other regulatory sequences and/or filler sequences and/or complements thereof, are heterologous to each other.

Constitutive Promoter: Promoters referred to herein as “constitutive promoters” actively promote transcription under most, but not necessarily all, environmental conditions and states of development or cell differentiation. Examples of constitutive promoters include the cauliflower mosaic virus (CaMV) 35S transcript initiation region and the 1′ or 2′ promoter derived from T-DNA of Agrobacterium tumefaciens, and other transcription initiation regions from various plant genes, such as the maize ubiquitin-1 promoter, known to those of skill.

Core Promoter: This is the minimal stretch of contiguous DNA sequence that is sufficient to direct accurate initiation of transcription by the RNA polymerase II machinery (for review see: Struhl, 1987, Cell 49: 295-297; Smale, 1994, In Transcription: Mechanisms and Regulation (eds R. C. Conaway and J. W. Conaway), pp 63-81/Raven Press, Ltd., New York; Smale, 1997, Biochim. Biophys. Acta 1351: 73-88; Smale et al., 1998, Cold Spring Harb. Symp. Quant. Biol. 58: 21-31; Smale, 2001, Genes & Dev. 15: 2503-2508; Weis and Reinberg, 1992, FASEB J. 6: 3300-3309; Burke et al., 1998, Cold Spring Harb. Symp. Quant. Biol 63: 75-82). There are several sequence motifs, including the TATA box, initiator (Inr), TFIIB recognition element (BRE) and downstream core promoter element (DPE), that are commonly found in core promoters, however not all of these elements occur in all promoters and there are no universal core promoter elements (Butler and Kadonaga, 2002, Genes & Dev. 16: 2583-2592).

Domain: Domains are fingerprints or signatures that can be used to characterize protein families and/or parts of proteins. Such fingerprints or signatures can comprise conserved (1) primary sequence, (2) secondary structure, and/or (3) three-dimensional conformation. A similar analysis can be applied to polynucleotides. Generally, each domain has been associated with either a conserved primary sequence or a sequence motif. Generally these conserved primary sequence motifs have been correlated with specific in vitro and/or in vivo activities. A domain can be any length, including the entirety of the polynucleotide to be transcribed. Examples of domains include, without limitation, AP2, helicase, homeobox, zinc finger, etc.

Endogenous: The term “endogenous,” within the context of the current invention refers to any polynucleotide, polypeptide or protein sequence which is a natural part of a cell or organisms regenerated from said cell. In the context of promoter, the term “endogenous coding region” or “endogenous cDNA” refers to the coding region that is naturally operably linked to the promoter.

Enhancer/Suppressor: An “enhancer” is a DNA regulatory element that can increase the steady state level of a transcript, usually by increasing the rate of transcription initiation. Enhancers usually exert their effect regardless of the distance, upstream or downstream location, or orientation of the enhancer relative to the start site of transcription. In contrast, a “suppressor” is a corresponding DNA regulatory element that decreases the steady state level of a transcript, again usually by affecting the rate of transcription initiation. The essential activity of enhancer and suppressor elements is to bind a protein factor(s). Such binding can be assayed, for example, by methods described below. The binding is typically in a manner that influences the steady state level of a transcript in a cell or in an in vitro transcription extract.

Exogenous: As referred to within, “exogenous” is any polynucleotide, polypeptide or protein sequence, whether chimeric or not, that is introduced into the genome of a host cell or organism regenerated from said host cell by any means other than by a sexual cross. Examples of means by which this can be accomplished are described below, and include Agrobacterium-mediated transformation (of dicots—e.g. Salomon et al. EMBO J. 3:141 (1984); Herrera-Estrella et al. EMBO J. 2:987 (1983); of monocots, representative papers are those by Escudero et al., Plant J. 10:355 (1996), Ishida et al., Nature Biotechnology 14:745 (1996), May et al., Bio/Technology 13:486 (1995)), biolistic methods (Armaleo et al., Current Genetics 17:97 1990)), electroporation, in planta techniques, and the like. Such a plant containing the exogenous nucleic acid is referred to here as a T₀ for the primary transgenic plant and T₁ for the first generation. The term “exogenous” as used herein is also intended to encompass inserting a naturally found element into a non-naturally found location.

Gene: The term “gene,” as used in the context of the current invention, encompasses all regulatory and coding sequence contiguously associated with a single hereditary unit with a genetic function (see SCHEMATIC 1). Genes can include non-coding sequences that modulate the genetic function that include, but are not limited to, those that specify polyadenylation, transcriptional regulation, DNA conformation, chromatin conformation, extent and position of base methylation and binding sites of proteins that control all of these. Genes encoding proteins are comprised of “exons” (coding sequences), which may be interrupted by “introns” (non-coding sequences). In some instances complexes of a plurality of protein or nucleic acids or other molecules, or of any two of the above, may be required for a gene's function. On the other hand a gene's genetic function may require only RNA expression or protein production, or may only require binding of proteins and/or nucleic acids without associated expression. In certain cases, genes adjacent to one another may share sequence in such a way that one gene will overlap the other. A gene can be found within the genome of an organism, in an artificial chromosome, in a plasmid, in any other sort of vector, or as a separate isolated entity.

Heterologous sequences: “Heterologous sequences” are those that are not operatively linked or are not contiguous to each other in nature. For example, a promoter from corn is considered heterologous to an Arabidopsis coding region sequence. Also, a promoter from a gene encoding a growth factor from corn is considered heterologous to a sequence encoding the corn receptor for the growth factor. Regulatory element sequences, such as UTRs or 3′ end termination sequences that do not originate in nature from the same gene as the coding sequence originates from, are considered heterologous to said coding sequence. Elements operatively linked in nature and contiguous to each other are not heterologous to each other.

Homologous: In the current invention, a “homologous” gene or polynucleotide or polypeptide refers to a gene or polynucleotide or polypeptide that shares sequence similarity with the gene or polynucleotide or polypeptide of interest. This similarity may be in only a fragment of the sequence and often represents a functional domain such as, examples including without limitation a DNA binding domain or a domain with tyrosine kinase activity. The functional activities of homologous polynucleotide are not necessarily the same.

Inducible Promoter: An “inducible promoter” in the context of the current invention refers to a promoter, the activity of which is influenced by certain conditions, such as light, temperature, chemical concentration, protein concentration, conditions in an organism, cell, or organelle, etc. A typical example of an inducible promoter, which can be utilized with the polynucleotides of the present invention, is PARSK1, the promoter from an Arabidopsis gene encoding a serine-threonine kinase enzyme, and which promoter is induced by dehydration, abscissic acid and sodium chloride (Wang and Goodman, Plant J. 8:37 (1995)). Examples of environmental conditions that may affect transcription by inducible promoters include anaerobic conditions, elevated temperature, the presence or absence of a nutrient or other chemical compound or the presence of light.

Modulate Transcription Level: As used herein, the phrase “modulate transcription” describes the biological activity of a promoter sequence or promoter control element. Such modulation includes, without limitation, includes up- and down-regulation of initiation of transcription, rate of transcription, and/or transcription levels.

Mutant: In the current invention, “mutant” refers to a heritable change in nucleotide sequence at a specific location. Mutant genes of the current invention may or may not have an associated identifiable phenotype.

Operable Linkage: An “operable linkage” is a linkage in which a promoter sequence or promoter control element is connected to a polynucleotide sequence (or sequences) in such a way as to place transcription of the polynucleotide sequence under the influence or control of the promoter or promoter control element. Two DNA sequences (such as a polynucleotide to be transcribed and a promoter sequence linked to the 5′ end of the polynucleotide to be transcribed) are said to be operably linked if induction of promoter function results in the transcription of mRNA encoding the polynucleotide and if the nature of the linkage between the two DNA sequences does not (1) result in the introduction of a frame-shift mutation, (2) interfere with the ability of the promoter sequence to direct the expression of the protein, antisense RNA or ribozyme, or (3) interfere with the ability of the DNA template to be transcribed. Thus, a promoter sequence would be operably linked to a polynucleotide sequence if the promoter was capable of effecting transcription of that polynucleotide sequence.

Optional Promoter Fragments: The phrase “optional promoter fragments” is used to refer to any sub-sequence of the promoter that is not required for driving transcription of an operationally linked coding region. These fragments comprise the 5′ UTR and any exon(s) of the endogenous coding region. The optional promoter fragments may also comprise any exon(s) and the 3′ or 5′ UTR of the gene residing upstream of the promoter (that is, 5′ to the promoter). Optional promoter fragments also include any intervening sequences that are introns or sequence that occurs between exons or an exon and the UTR.

Orthologous: “Orthologous” is a term used herein to describe a relationship between two or more polynucleotides or proteins. Two polynucleotides or proteins are “orthologous” to one another if they serve a similar function in different organisms. In general, orthologous polynucleotides or proteins will have similar catalytic functions (when they encode enzymes) or will serve similar structural functions (when they encode proteins or RNA that form part of the ultrastructure of a cell).

Percentage of sequence identity: “Percentage of sequence identity,” as used herein, is determined by comparing two optimally aligned sequences over a comparison window, where the fragment of the polynucleotide or amino acid sequence in the comparison window may comprise additions or deletions (e.g., gaps or overhangs) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by inspection. Given that two sequences have been identified for comparison, GAP and BESTFIT are preferably employed to determine their optimal alignment. Typically, the default values of 5.00 for gap weight and 0.30 for gap weight length are used.

Plant Promoter: A “plant promoter” is a promoter capable of initiating transcription in plant cells and can modulate transcription of a polynucleotide. Such promoters need not be of plant origin. For example, promoters derived from plant viruses, such as the CaMV35S promoter or from Agrobacterium tumefaciens such as the T-DNA promoters, can be plant promoters. A typical example of a plant promoter of plant origin is the maize ubiquitin-1 (ubi-1) promoter known to those of skill.

Plant Tissue: The term “plant tissue” includes differentiated and undifferentiated tissues or plants, including but not limited to roots, stems, shoots, cotyledons, epicotyl, hypocotyl, leaves, pollen, seeds, tumor tissue and various forms of cells in culture such as single cells, protoplast, embryos, and callus tissue. The plant tissue may be in plants or in organ, tissue or cell culture.

Preferential Transcription: “Preferential transcription” is defined as transcription that occurs in a particular pattern of cell types or developmental times or in response to specific stimuli or combination thereof. Non-limitive examples of preferential transcription include: high transcript levels of a desired sequence in root tissues; detectable transcript levels of a desired sequence in certain cell types during embryogenesis; and low transcript levels of a desired sequence under drought conditions. Such preferential transcription can be determined by measuring initiation, rate, and/or levels of transcription.

Promoter: A “promoter” is a DNA sequence that directs the transcription of a polynucleotide. Typically a promoter is located in the 5′ region of a polynucleotide to be transcribed, proximal to the transcriptional start site of such polynucleotide. More typically, promoters are defined as the region upstream of the first exon; more typically, as a region upstream of the first of multiple transcription start sites; more typically, as the region downstream of the preceding gene and upstream of the first of multiple transcription start sites; more typically, the region downstream of the polyA signal and upstream of the first of multiple transcription start sites; even more typically, about 3,000 nucleotides upstream of the ATG of the first exon; even more typically, 2,000 nucleotides upstream of the first of multiple transcription start sites. The promoters of the invention comprise at least a core promoter as defined above. Frequently promoters are capable of directing transcription of genes located on each of the complementary DNA strands that are 3′ to the promoter. Stated differently, many promoters exhibit bidirectionality and can direct transcription of a downstream gene when present in either orientation (i.e. 5′ to 3′ or 3′ to 5′ relative to the coding region of the gene). Additionally, the promoter may also include at least one control element such as an upstream element. Such elements include UARs and optionally, other DNA sequences that affect transcription of a polynucleotide such as a synthetic upstream element.

Promoter Control Element: The term “promoter control element” as used herein describes elements that influence the activity of the promoter. Promoter control elements include transcriptional regulatory sequence determinants such as, but not limited to, enhancers, scaffold/matrix attachment regions, TATA boxes, transcription start locus control regions, UARs, URRs, other transcription factor binding sites and inverted repeats.

Public sequence: The term “public sequence,” as used in the context of the instant application, refers to any sequence that has been deposited in a publicly accessible database prior to the filing date of the present application. This term encompasses both amino acid and nucleotide sequences. Such sequences are publicly accessible, for example, on the BLAST databases on the NCBI FTP web site (accessible at ncbi.nlm.nih.gov/ftp). The database at the NCBI FTP site utilizes “gi” numbers assigned by NCBI as a unique identifier for each sequence in the databases, thereby providing a non-redundant database for sequence from various databases, including GenBank, EMBL, DBBJ, (DNA Database of Japan) and PDB (Brookhaven Protein Data Bank).

Regulatory Sequence: The term “regulatory sequence,” as used in the current invention, refers to any nucleotide sequence that influences transcription or translation initiation and rate, or stability and/or mobility of a transcript or polypeptide product. Regulatory sequences include, but are not limited to, promoters, promoter control elements, protein binding sequences, 5′ and 3′ UTRs, transcriptional start sites, termination sequences, polyadenylation sequences, introns, certain sequences within amino acid coding sequences such as secretory signals, protease cleavage sites, etc.

Related Sequences: “Related sequences” refer to either a polypeptide or a nucleotide sequence that exhibits some degree of sequence similarity with a reference sequence.

Specific Promoters: In the context of the current invention, “specific promoters” refers to a subset of promoters that have a high preference for modulating transcript levels in a specific tissue or organ or cell and/or at a specific time during development of an organism. By “high preference” is meant at least 3-fold, preferably 5-fold, more preferably at least 10-fold still more preferably at least 20-fold, 50-fold or 100-fold increase in transcript levels under the specific condition over the transcription under any other reference condition considered. Typical examples of temporal and/or tissue or organ specific promoters of plant origin that can be used with the polynucleotides of the present invention, are: PTA29, a promoter which is capable of driving gene transcription specifically in tapetum and only during anther development (Koltonow et al., Plant Cell 2:1201 (1990); RCc2 and RCc3, promoters that direct root-specific gene transcription in rice (Xu et al., Plant Mol. Biol. 27:237 (1995); TobRB27, a root-specific promoter from tobacco (Yamamoto et al., Plant Cell 3:371 (1991)). Examples of tissue-specific promoters under developmental control include promoters that initiate transcription only in certain tissues or organs, such as root, ovule, fruit, seeds, or flowers. Other specific promoters include those from genes encoding seed storage proteins or the lipid body membrane protein, oleosin. A few root-specific promoters are noted above. See also “Preferential transcription”.

Stringency: “Stringency” as used herein is a function of probe length, probe composition (G+C content), and salt concentration, organic solvent concentration, and temperature of hybridization or wash conditions. Stringency is typically compared by the parameter Tₘ, which is the temperature at which 50% of the complementary molecules in the hybridization are hybridized, in terms of a temperature differential from Tₘ. High stringency conditions are those providing a condition of Tₘ-5° C. to Tₘ-10° C. Medium or moderate stringency conditions are those providing Tₘ-20° C. to Tₘ-29° C. Low stringency conditions are those providing a condition of Tₘ-40° C. to Tₘ-48° C. The relationship of hybridization conditions to Tₘ (in ° C.) is expressed in the mathematical equation

Tₘ=81.5−16.6(log₁₀[Na⁺])+0.41(% G+C)−(600/N) (1)

where N is the length of the probe. This equation works well for probes 14 to 70 nucleotides in length that are identical to the target sequence. The equation below for Tₘ of DNA-DNA hybrids is useful for probes in the range of 50 to greater than 500 nucleotides, and for conditions that include an organic solvent (formamide).

Tₘ=81.5+16.6 log {[Na⁺]/(1+0.7[Na⁺])}+0.41(% G+C)−500/L 0.63(% formamide) (2)

where L is the length of the probe in the hybrid. (P. Tijessen, “Hybridization with Nucleic Acid Probes” in Laboratory Techniques in Biochemistry and Molecular Biology, P. C. vand der Vliet, ed., c. 1993 by Elsevier, Amsterdam.) The Tₘ of equation (2) is affected by the nature of the hybrid; for DNA-RNA hybrids Tₘ is 10-15° C. higher than calculated, for RNA-RNA hybrids Tₘ is 20-25° C. higher. Because the Tₘ decreases about 1° C. for each 1% decrease in homology when a long probe is used (Bonner et al., J. Mol. Biol. 81:123 (1973)), stringency conditions can be adjusted to favor detection of identical genes or related family members.

Equation (2) is derived assuming equilibrium and therefore, hybridizations according to the present invention are most preferably performed under conditions of probe excess and for sufficient time to achieve equilibrium. The time required to reach equilibrium can be shortened by inclusion of a hybridization accelerator such as dextran sulfate or another high volume polymer in the hybridization buffer.

Stringency can be controlled during the hybridization reaction or after hybridization has occurred by altering the salt and temperature conditions of the wash solutions used. The formulas shown above are equally valid when used to compute the stringency of a wash solution. Preferred wash solution stringencies lie within the ranges stated above; high stringency is 5-8° C. below Tₘ, medium or moderate stringency is 26-29° C. below Tₘ and low stringency is 45-48° C. below Tₘ.

Substantially free of: A composition containing A is “substantially free of” B when at least 85% by weight of the total A+B in the composition is A. Preferably, A comprises at least about 90% by weight of the total of A+B in the composition, more preferably at least about 95% or even 99% by weight. For example, a plant gene can be substantially free of other plant genes. Other examples include, but are not limited to, ligands substantially free of receptors (and vice versa), a growth factor substantially free of other growth factors and a transcription binding factor substantially free of nucleic acids.

Suppressor: See “Enhancer/Suppressor”

TATA to start: “TATA to start” shall mean the distance, in number of nucleotides, between the primary TATA motif and the start of transcription.

Transgenic plant: A “transgenic plant” is a plant having one or more plant cells that contain at least one exogenous polynucleotide introduced by recombinant nucleic acid methods.

Translational start site: In the context of the present invention, a “translational start site” is usually an ATG or AUG in a transcript, often the first ATG or AUG. A single protein encoding transcript, however, may have multiple translational start sites.

Transcription start site: “Transcription start site” is used in the current invention to describe the point at which transcription is initiated. This point is typically located about 25 nucleotides downstream from a TFIID binding site, such as a TATA box. Transcription can initiate at one or more sites within the gene, and a single polynucleotide to be transcribed may have multiple transcriptional start sites, some of which may be specific for transcription in a particular cell-type or tissue or organ. “+1” is stated relative to the transcription start site and indicates the first nucleotide in a transcript.

Upstream Activating Region (UAR): An “Upstream Activating Region” or “UAR” is a position or orientation dependent nucleic acid element that primarily directs tissue, organ, cell type, or environmental regulation of transcript level, usually by affecting the rate of transcription initiation. Corresponding DNA elements that have a transcription inhibitory effect are called herein “Upstream Repressor Regions” or “URR”s. The essential activity of these elements is to bind a protein factor. Such binding can be assayed by methods described below. The binding is typically in a manner that influences the steady state level of a transcript in a cell or in vitro transcription extract.

Untranslated region (UTR): A “UTR” is any contiguous series of nucleotide bases that is transcribed, but is not translated. A 5′ UTR lies between the start site of the transcript and the translation initiation codon and includes the +1 nucleotide. A 3′ UTR lies between the translation termination codon and the end of the transcript. UTRs can have particular functions such as increasing mRNA message stability or translation attenuation. Examples of 3′ UTRs include, but are not limited to polyadenylation signals and transcription termination sequences.

Variant: The term “variant” is used herein to denote a polypeptide or protein or polynucleotide molecule that differs from others of its kind in some way. For example, polypeptide and protein variants can consist of changes in amino acid sequence and/or charge and/or post-translational modifications (such as glycosylation, etc). Likewise, polynucleotide variants can consist of changes that add or delete a specific UTR or exon sequence. It will be understood that there may be sequence variations within sequence or fragments used or disclosed in this application. Preferably, variants will be such that the sequences have at least 80%, preferably at least 90%, 95, 97, 98, or 99% sequence identity. Variants preferably measure the primary biological function of the native polypeptide or protein or polynucleotide.

### 2. Introduction

The polynucleotides of the invention comprise promoters and promoter control elements that are capable of modulating transcription.

Such promoters and promoter control elements can be used in combination with native or heterologous promoter fragments, control elements or other regulatory sequences to modulate transcription and/or translation.

Specifically, promoters and control elements of the invention can be used to modulate transcription of a desired polynucleotide, which includes without limitation:
(a) antisense;
(b) ribozymes;
(c) coding sequences; or
(d) fragments thereof.
   The promoter also can modulate transcription in a host genome in cis- or in trans-.

In an organism, such as a plant, the promoters and promoter control elements of the instant invention are useful to produce preferential transcription which results in a desired pattern of transcript levels in a particular cells, tissues, or organs, or under particular conditions.

### 3. Identifying and Isolating Promoter Sequences of the Invention

The promoters and promoter control elements of the present invention are presented in Table 1 in the section entitled “The predicted promoter” sequence and were identified from Arabidopsis thaliana or Oryza sativa. Additional promoter sequences encompassed by the invention can be identified as described below.

The promoter control elements of the present invention include those that comprise a sequence shown in Table 1 in the section entitled “The predicted promoter sequence” and fragments thereof. The size of the fragments of the row titled “The predicted promoter sequence” can range from 5 bases to 10 kilobases (kb). Typically, the fragment size is no smaller than 8 bases; more typically, no smaller than 12; more typically, no smaller than 15 bases; more typically, no smaller than 20 bases; more typically, no smaller than 25 bases; even more typically, no more than 30, 35, 40 or 50 bases.

Usually, the fragment size in no larger than 5 kb bases; more usually, no larger than 2 kb; more usually, no larger than 1 kb; more usually, no larger than 800 bases; more usually, no larger than 500 bases; even more usually, no more than 250, 200, 150 or 100 bases.

3.1 Cloning Methods

Isolation from genomic libraries of polynucleotides comprising the sequences of the promoters and promoter control elements of the present invention is possible using known techniques.

For example, polymerase chain reaction (PCR) can amplify the desired polynucleotides utilizing primers designed from sequences in the row titled “The spatial expression of the promoter-marker-vector”. Polynucleotide libraries comprising genomic sequences can be constructed according to Sambrook et al., Molecular Cloning: A Laboratory Manual, 2^{nd} Ed. (1989) Cold Spring Harbor Press, Cold Spring Harbor, N.Y.), for example.

Other procedures for isolating polynucleotides comprising the promoter sequences of the invention include, without limitation, tail-PCR, and 5′ rapid amplification of cDNA ends (RACE). See, for tail-PCR, for example, Liu et al., Plant J 8(3): 457-463 (September 1995); Liu et al., Genomics 25: 674-681 (1995); Liu et al., Nucl. Acids Res. 21(14): 3333-3334 (1993); and Zoe et al., BioTechniques 27(2): 240-248 (1999); for RACE, see, for example, PCR Protocols: A Guide to Methods and Applications, (1990) Academic Press, Inc.

3.2 Chemical Synthesis

In addition, the promoters and promoter control elements described in Table 1 in the section entitled “The predicted promoter” sequence can be chemically synthesized according to techniques in common use. See, for example, Beaucage et al., Tet. Lett. (1981) 22: 1859 and U.S. Pat. No. 4,668,777.

Such chemical oligonucleotide synthesis can be carried out using commercially available devices, such as, Biosearch 4600 or 8600 DNA synthesizer, by Applied Biosystems, a division of Perkin-Elmer Corp., Foster City, Calif., USA; and Expedite by Perceptive Biosystems, Framingham, Mass., USA.

Synthetic RNA, including natural and/or analog building blocks, can be synthesized on the Biosearch 8600 machines, see above.

Oligonucleotides can be synthesized and then ligated together to construct the desired polynucleotide.

### 4. Generating Reduced and “Core” Promoter Sequences

Included in the present invention are reduced and “core” promoter sequences. The reduced promoters can be isolated from the promoters of the invention by deleting at least one 5′ UTR, exon or 3′ UTR sequence present in the promoter sequence that is associated with a gene or coding region located 5′ to the promoter sequence or in the promoter's endogenous coding region.

Similarly, the “core” promoter sequences can be generated by deleting all 5′ UTRs, exons and 3′ UTRs present in the promoter sequence and the associated intervening sequences that are related to the gene or coding region 5′ to the promoter region and the promoter's endogenous coding region.

This data is presented in the row titled “Optional Promoter Fragments”.

### 5. Isolating Related Promoter Sequences

Included in the present invention are promoter and promoter control elements that are related to those described in Table 1 in the section entitled “The predicted promoter sequence”. Such related sequence can be isolated utilizing
(a) nucleotide sequence identity;
(b) coding sequence identity; or
(c) common function or gene products.
   Relatives can include both naturally occurring promoters and non-natural promoter sequences. Non-natural related promoters include nucleotide substitutions, insertions or deletions of naturally-occurring promoter sequences that do not substantially affect transcription modulation activity. For example, the binding of relevant DNA binding proteins can still occur with the non-natural promoter sequences and promoter control elements of the present invention.

According to current knowledge, promoter sequences and promoter control elements exist as functionally important regions, such as protein binding sites, and spacer regions. These spacer regions are apparently required for proper positioning of the protein binding sites. Thus, nucleotide substitutions, insertions and deletions can be tolerated in these spacer regions to a certain degree without loss of function.

In contrast, less variation is permissible in the functionally important regions, since changes in the sequence can interfere with protein binding. Nonetheless, some variation in the functionally important regions is permissible so long as function is conserved.

The effects of substitutions, insertions and deletions to the promoter sequences or promoter control elements may be to increase or decrease the binding of relevant DNA binding proteins to modulate transcript levels of a polynucleotide to be transcribed. Effects may include tissue-specific or condition-specific modulation of transcript levels of the polypeptide to be transcribed. Polynucleotides representing changes to the nucleotide sequence of the DNA-protein contact region by insertion of additional nucleotides, changes to identity of relevant nucleotides, including use of chemically-modified bases, or deletion of one or more nucleotides are considered encompassed by the present invention.

5.1 Relatives Based on Nucleotide Sequence Identity

Included in the present invention are promoters exhibiting nucleotide sequence identity to those described in Table 1 in the section entitled “The predicted promoter sequence”.

5.1.1 Definition

Typically, such related promoters exhibit at least 80% sequence identity, preferably at least 85%, more preferably at least 90%, and most preferably at least 95%, even more preferably, at least 96%, 97%, 98% or 99% sequence identity compared to those shown in Table 1 in the section entitled “The predicted promoter” sequence. Such sequence identity can be calculated by the algorithms and computers programs described above.

Usually, such sequence identity is exhibited in an alignment region that is at least 75% of the length of a sequence shown in Table 1 in the section entitled “The predicted promoter” sequence or corresponding full-length sequence; more usually at least 80%; more usually, at least 85%, more usually at least 90%, and most usually at least 95%, even more usually, at least 96%, 97%, 98% or 99% of the length of a sequence shown in Table 1 in the section entitled “The predicted promoter sequence”.

The percentage of the alignment length is calculated by counting the number of residues of the sequence in region of strongest alignment, e.g., a continuous region of the sequence that contains the greatest number of residues that are identical to the residues between two sequences that are being aligned. The number of residues in the region of strongest alignment is divided by the total residue length of a sequence in Table 1 in the section entitled “The predicted promoter sequence”.

These related promoters may exhibit similar preferential transcription as those promoters described in Table 1 in the section entitled “The predicted promoter sequence”.

5.1.2 Construction of Polynucleotides

Naturally occurring promoters that exhibit nucleotide sequence identity to those shown in Table 1 in the section entitled “The predicted promoter sequence” can be isolated using the techniques as described above. More specifically, such related promoters can be identified by varying stringencies, as defined above, in typical hybridization procedures such as Southern blots or probing of polynucleotide libraries, for example.

Non-natural promoter variants of those shown in Table 1 can be constructed using cloning methods that incorporate the desired nucleotide variation. See, for example, Ho, S. N., et al. Gene 77:51-59 1989, describing a procedure site directed mutagenesis using PCR.

Any related promoter showing sequence identity to those shown in Table can be chemically synthesized as described above.

Also, the present invention includes non-natural promoters that exhibit the above-sequence identity to those in Table 1.

The promoters and promoter control elements of the present invention may also be synthesized with 5′ or 3′ extensions, to facilitate additional manipulation, for instance.

The present invention also includes reduced promoter sequences. These sequences have at least one of the optional promoter fragments deleted.

Core promoter sequences are another embodiment of the present invention. The core promoter sequences have all of the optional promoter fragments deleted.

### 6. Testing of Polynucleotides

Polynucleotides of the invention were tested for activity by cloning the sequence into an appropriate vector, transforming plants with the construct and assaying for marker gene expression. Recombinant DNA constructs were prepared which comprise the polynucleotide sequences of the invention inserted into a vector suitable for transformation of plant cells. The construct can be made using standard recombinant DNA techniques (Sambrook et al. 1989) and can be introduced to the species of interest by Agrobacterium-mediated transformation or by other means of transformation as referenced below.

The vector backbone can be any of those typical in the art such as plasmids, viruses, artificial chromosomes, BACs, YACs and PACs and vectors of the sort described by
(a) BAC: Shizuya et al., Proc. Natl. Acad. Sci. USA 89: 8794-8797 (1992); Hamilton et al., Proc. Natl. Acad. Sci. USA 93: 9975-9979 (1996);
(b) YAC: Burke et al., Science 236:806-812 (1987);
(c) PAC: Sternberg N. et al., Proc Natl Acad Sci USA. January; 87(1):103-7 (1990);
(d) Bacteria-Yeast Shuttle Vectors: Bradshaw et al., Nucl Acids Res 23: 4850-4856 (1995);
(e) Lambda Phage Vectors: Replacement Vector, e.g., Frischauf et al., J. Mol Biol 170: 827-842 (1983); or Insertion vector, e.g., Huynh et al., In: Glover N M (ed) DNA Cloning: A practical Approach, Vol. 1 Oxford: IRL Press (1985); T-DNA gene fusion vectors: Walden et al., Mol Cell Biol 1: 175-194 (1990); and
(g) Plasmid vectors: Sambrook et al., infra.

Typically, the construct comprises a vector containing a sequence of the present invention operationally linked to any marker gene. The polynucleotide was identified as a promoter by the expression of the marker gene. Although many marker genes can be used, Green Fluroescent Protein (GFP) is preferred. The vector may also comprise a marker gene that confers a selectable phenotype on plant cells. The marker may encode biocide resistance, particularly antibiotic resistance, such as resistance to kanamycin, G418, bleomycin, hygromycin, or herbicide resistance, such as resistance to chlorosulfuron or phosphinotricin. Vectors can also include origins of replication, scaffold attachment regions (SARs), markers, homologous sequences, introns, etc.

### 7. Promoter Control Element Configuration

A common configuration of the promoter control elements in RNA polymerase II promoters is shown below:

For more description, see, for example, “Models for prediction and recognition of eukaryotic promoters”, T. Werner, Mammalian Genome, 10, 168-175 (1999).

Promoters are generally modular in nature. Promoters can consist of a basal promoter which functions as a site for assembly of a transcription complex comprising an RNA polymerase, for example RNA polymerase II. A typical transcription complex will include additional factors such as TF_{II}B, TF_{II}D, and TF_{II}E. Of these, TF_{II}D appears to be the only one to bind DNA directly. The promoter might also contain one or more promoter control elements such as the elements discussed above. These additional control elements may function as binding sites for additional transcription factors that have the function of modulating the level of transcription with respect to tissue specificity and of transcriptional responses to particular environmental or nutritional factors, and the like.

One type of promoter control element is a polynucleotide sequence representing a binding site for proteins. Typically, within a particular functional module, protein binding sites constitute regions of 5 to 60, preferably 10 to 30, more preferably 10 to 20 nucleotides. Within such binding sites, there are typically 2 to 6 nucleotides which specifically contact amino acids of the nucleic acid binding protein.

The protein binding sites are usually separated from each other by 10 to several hundred nucleotides, typically by 15 to 150 nucleotides, often by 20 to 50 nucleotides.

Further, protein binding sites in promoter control elements often display dyad symmetry in their sequence. Such elements can bind several different proteins, and/or a plurality of sites can bind the same protein. Both types of elements may be combined in a region of 50 to 1,000 base pairs.

Binding sites for any specific factor have been known to occur almost anywhere in a promoter. For example, functional AP-1 binding sites can be located far upstream, as in the rat bone sialoprotein gene, where an AP-1 site located about 900 nucleotides upstream of the transcription start site suppresses expression. Yamauchi et al., Matrix Biol., 15, 119-130 (1996). Alternatively, an AP-1 site located close to the transcription start site plays an important role in the expression of Moloney murine leukemia virus. Sap et al., Nature, 340, 242-244, (1989).

8. Constructing Promoters with Control Elements

8.1 Combining Promoters and Promoter Control Elements

The promoter polynucleotides and promoter control elements of the present invention, both naturally occurring and synthetic, can be combined with each other to produce the desired preferential transcription. Also, the polynucleotides of the invention can be combined with other known sequences to obtain other useful promoters to modulate, for example, tissue transcription specific or transcription specific to certain conditions. Such preferential transcription can be determined using the techniques or assays described above.

Fragments, variants, as well as full-length sequences those shown in Table 1 in the section entitled “The predicted promoter sequence” and relatives are useful alone or in combination.

The location and relation of promoter control elements within a promoter can affect the ability of the promoter to modulate transcription. The order and spacing of control elements is a factor when constructing promoters.

Non-natural control elements can be constructed by inserting, deleting or substituting nucleotides into the promoter control elements described above. Such control elements are capable of transcription modulation that can be determined using any of the assays described above.

8.2 Number of Promoter Control Elements

Promoters can contain any number of control elements. For example, a promoter can contain multiple transcription binding sites or other control elements. One element may confer tissue or organ specificity; another element may limit transcription to specific time periods, etc. Typically, promoters will contain at least a basal or core promoter as described above. Any additional element can be included as desired. For example, a fragment comprising a basal or “core” promoter can be fused with another fragment with any number of additional control elements.

8.3 Spacing Between Control Elements

Spacing between control elements or the configuration or control elements can be determined or optimized to permit the desired protein-polynucleotide or polynucleotide interactions to occur.

For example, if two transcription factors bind to a promoter simultaneously or relatively close in time, the binding sites are spaced to allow each factor to bind without steric hinderance. The spacing between two such hybridizing control elements can be as small as a profile of a protein bound to a control element. In some cases, two protein binding sites can be adjacent to each other when the proteins bind at different times during the transcription process.

Further, when two control elements hybridize the spacing between such elements will be sufficient to allow the promoter polynucleotide to hairpin or loop to permit the two elements to bind. The spacing between two such hybridizing control elements can be as small as a t-RNA loop, to as large as 10 kb.

Typically, the spacing is no smaller than 5 bases; more typically, no smaller than 8; more typically, no smaller than 15 bases; more typically, no smaller than 20 bases; more typically, no smaller than 25 bases; even more typically, no more than 30, 35, 40 or 50 bases.

Usually, the fragment size in no larger than 5 kb bases; more usually, no larger than 2 kb; more usually, no larger than 1 kb; more usually, no larger than 800 bases; more usually, no larger than 500 bases; even more usually, no more than 250, 200, 150 or 100 bases.

Such spacing between promoter control elements can be determined using the techniques and assays described above.

8.4 Other Promoters

The following are promoters that are induced under stress conditions and can be combined with those of the present invention: ldhl (oxygen stress; tomato; see Germain and Ricard. 1997. Plant Mol Biol 35:949-54), GPx and CAT (oxygen stress; mouse; see Franco et al. 1999. Free Radic Biol Med 27:1122-32), ci7 (cold stress; potato; see Kirch et al. 1997. Plant Mol Biol. 33:897-909), Bz2 (heavy metals; maize; see Marrs and Walbot. 1997. Plant Physiol 113:93-102), HSP32 (hyperthermia; rat; see Raju and Maines. 1994. Biochim Biophys Acta 1217:273-80); MAPKAPK-2 (heat shock; Drosophila; see Larochelle and Suter. 1995. Gene 163:209-14).

In addition, the following examples of promoters are induced by the presence or absence of light can be used in combination with those of the present invention: Topoisomerase II (pea; see Reddy et al. 1999. Plant Mol Biol 41:125-37), chalcone synthase (soybean; see Wingender et al. 1989. Mol Gen Genet 218:315-22) mdm2 gene (human tumor; see Saucedo et al. 1998. Cell Growth Differ 9:119-30), Clock and BMAL1 (rat; see Namihira et al. 1999. Neurosci Lett 271:1-4, PHYA (Arabidopsis; see Canton and Quail 1999. Plant Physiol 121:1207-16), PRB-1b (tobacco; see Sessa et al. 1995. Plant Mol Biol 28:537-47) and Ypr10 (common bean; see Walter et al. 1996. Eur J Biochem 239:281-93).

The promoters and control elements of the following genes can be used in combination with the present invention to confer tissue specificity: MipB (iceplant; Yamada et al. 1995. Plant Cell 7:1129-42) and SUCS (root nodules; broadbean; Kuster et al. 1993. Mol Plant Microbe Interact 6:507-14) for roots, OsSUT1 (rice; Hirose et al. 1997. Plant Cell Physiol 38:1389-96) for leaves, Msg (soybean; Stomvik et al. 1999. Plant Mol Biol 41:217-31) for siliques, cell (Arabidopsis; Shani et al. 1997. Plant Mol Biol 34(6):837-42) and ACT11 (Arabidopsis; Huang et al. 1997. Plant Mol Biol 33:125-39) for inflorescence.

Still other promoters are affected by hormones or participate in specific physiological processes, which can be used in combination with those of present invention. Some examples are the ACC synthase gene that is induced differently by ethylene and brassinosteroids (mung bean; Yi et al. 1999. Plant Mol Biol 41:443-54), the TAPG1 gene that is active during abscission (tomato; Kalaitzis et al. 1995. Plant Mol Biol 28:647-56), and the 1-aminocyclopropane-1-carboxylate synthase gene (carnation; Jones et al. 19951 Plant Mol Biol 28:505-12) and the CP-2/cathepsin L gene (rat; Kim and Wright. 1997. Biol Reprod 57:1467-77), both active during senescence.

### 9. Vectors

Vectors are a useful component of the present invention. In particular, the present promoters and/or promoter control elements may be delivered to a system such as a cell by way of a vector. For the purposes of this invention, such delivery may range from simply introducing the promoter or promoter control element by itself randomly into a cell to integration of a cloning vector containing the present promoter or promoter control element. Thus, a vector need not be limited to a DNA molecule such as a plasmid, cosmid or bacterial phage that has the capability of replicating autonomously in a host cell. All other manner of delivery of the promoters and promoter control elements of the invention are envisioned. The various T-DNA vector types are a preferred vector for use with the present invention. Many useful vectors are commercially available.

It may also be useful to attach a marker sequence to the present promoter and promoter control element in order to determine activity of such sequences. Marker sequences typically include genes that provide antibiotic resistance, such as tetracycline resistance, hygromycin resistance or ampicillin resistance, or provide herbicide resistance. Specific selectable marker genes may be used to confer resistance to herbicides such as glyphosate, glufosinate or broxynil (Comai et al., Nature 317: 741-744 (1985); Gordon-Kamm et al., Plant Cell 2: 603-618 (1990); and Stalker et al., Science 242: 419-423 (1988)). Other marker genes exist which provide hormone responsiveness.

9.1 Modification of Transcription by Promoters and Promoter Control Elements

The promoter or promoter control element of the present invention may be operably linked to a polynucleotide to be transcribed. In this manner, the promoter or promoter control element may modify transcription by modulate transcript levels of that polynucleotide when inserted into a genome.

However, prior to insertion into a genome, the promoter or promoter control element need not be linked, operably or otherwise, to a polynucleotide to be transcribed. For example, the promoter or promoter control element may be inserted alone into the genome in front of a polynucleotide already present in the genome. In this manner, the promoter or promoter control element may modulate the transcription of a polynucleotide that was already present in the genome. This polynucleotide may be native to the genome or inserted at an earlier time.

Alternatively, the promoter or promoter control element may be inserted into a genome alone to modulate transcription. See, for example, Vaucheret, H et al. (1998) Plant J 16: 651-659. Rather, the promoter or promoter control element may be simply inserted into a genome or maintained extrachromosomally as a way to divert transcription resources of the system to itself. This approach may be used to downregulate the transcript levels of a group of polynucleotide(s).

9.2 Polynucleotide to be Transcribed

The nature of the polynucleotide to be transcribed is not limited. Specifically, the polynucleotide may include sequences that will have activity as RNA as well as sequences that result in a polypeptide product. These sequences may include, but are not limited to antisense sequences, ribozyme sequences, spliceosomes, amino acid coding sequences, and fragments thereof.

Specific coding sequences may include, but are not limited to endogenous proteins or fragments thereof, or heterologous proteins including marker genes or fragments thereof.

Promoters and control elements of the present invention are useful for modulating metabolic or catabolic processes. Such processes include, but are not limited to, secondary product metabolism, amino acid synthesis, seed protein storage, oil development, pest defense and nitrogen usage. Some examples of genes, transcripts and peptides or polypeptides participating in these processes, which can be modulated by the present invention: are tryptophan decarboxylase (tdc) and strictosidine synthase (str1), dihydrodipicolinate synthase (DHDPS) and aspartate kinase (AK), 2S albumin and alpha-, beta-, and gamma-zeins, ricinoleate and 3-ketoacyl-ACP synthase (KAS), Bacillus thuringiensis (Bt) insecticidal protein, cowpea trypsin inhibitor (CpTI), asparagine synthetase and nitrite reductase. Alternatively, expression constructs can be used to inhibit expression of these peptides and polypeptides by incorporating the promoters in constructs for antisense use, co-suppression use or for the production of dominant negative mutations.

9.3 Other Regulatory Elements

As explained above, several types of regulatory elements exist concerning transcription regulation. Each of these regulatory elements may be combined with the present vector if desired.

9.4 Other Components of Vectors

Translation of eukaryotic mRNA is often initiated at the codon that encodes the first methionine. Thus, when constructing a recombinant polynucleotide according to the present invention for expressing a protein product, it is preferable to ensure that the linkage between the 3′ portion, preferably including the TATA box, of the promoter and the polynucleotide to be transcribed, or a functional derivative thereof, does not contain any intervening codons which are capable of encoding a methionine.

The vector of the present invention may contain additional components. For example, an origin of replication allows for replication of the vector in a host cell. Additionally, homologous sequences flanking a specific sequence allows for specific recombination of the specific sequence at a desired location in the target genome. T-DNA sequences also allow for insertion of a specific sequence randomly into a target genome.

The vector may also be provided with a plurality of restriction sites for insertion of a polynucleotide to be transcribed as well as the promoter and/or promoter control elements of the present invention. The vector may additionally contain selectable marker genes. The vector may also contain a transcriptional and translational initiation region, and a transcriptional and translational termination region functional in the host cell. The termination region may be native with the transcriptional initiation region, may be native with the polynucleotide to be transcribed, or may be derived from another source. Convenient termination regions are available from the Ti-plasmid of A. tumefaciens, such as the octopine synthase and nopaline synthase termination regions. See also, Guerineau et al., (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. 1989) Nucleic Acids Res. 17:7891-7903; Joshi et al. (1987) Nucleic Acid Res. 15:9627-9639.

Where appropriate, the polynucleotide to be transcribed may be optimized for increased expression in a certain host cell. For example, the polynucleotide can be synthesized using preferred codons for improved transcription and translation. See U.S. Pat. Nos. 5,380,831, 5,436,391; see also and Murray et al., (1989) Nucleic Acids Res. 17:477-498.

Additional sequence modifications include elimination of sequences encoding spurious polyadenylation signals, exon intron splice site signals, transposon-like repeats, and other such sequences well characterized as deleterious to expression. The G−C content of the polynucleotide may be adjusted to levels average for a given cellular host, as calculated by reference to known genes expressed in the host cell. The polynucleotide sequence may be modified to avoid hairpin secondary mRNA structures.

A general description of expression vectors and reporter genes can be found in Gruber, et al., “Vectors for Plant Transformation, in Methods in Plant Molecular Biology & Biotechnology” in Glich et al., (Eds. pp. 89-119, CRC Press, 1993). Moreover GUS expression vectors and GUS gene cassettes are available from Clonetech Laboratories, Inc., Palo Alto, Calif. while luciferase expression vectors and luciferase gene cassettes are available from Promega Corp. (Madison, Wis.). GFP vectors are available from Aurora Biosciences.

### 10. Polynucleotide Insertion Into a Host Cell

The polynucleotides according to the present invention can be inserted into a host cell. A host cell includes but is not limited to a plant, mammalian, insect, yeast, and prokaryotic cell, preferably a plant cell.

The method of insertion into the host cell genome is chosen based on convenience. For example, the insertion into the host cell genome may either be accomplished by vectors that integrate into the host cell genome or by vectors which exist independent of the host cell genome.

10.1 Polynucleotides Autonomous of the Host Genome

The polynucleotides of the present invention can exist autonomously or independent of the host cell genome. Vectors of these types are known in the art and include, for example, certain type of non-integrating viral vectors, autonomously replicating plasmids, artificial chromosomes, and the like.

Additionally, in some cases transient expression of a polynucleotide may be desired.

10.2 Polynucleotides Integrated Into the Host Genome

The promoter sequences, promoter control elements or vectors of the present invention may be transformed into host cells. These transformations may be into protoplasts or intact tissues or isolated cells. Preferably expression vectors are introduced into intact tissue. General methods of culturing plant tissues are provided for example by Maki et al. “Procedures for Introducing Foreign DNA into Plants” in Methods in Plant Molecular Biology & Biotechnology, Glich et al. (Eds. pp. 67-88 CRC Press, 1993); and by Phillips et al. “Cell-Tissue Culture and In-Vitro Manipulation” in Corn & Corn Improvement, 3rd Edition 10 Sprague et al. (Eds. pp. 345-387) American Society of Agronomy Inc. et al. 1988.

Methods of introducing polynucleotides into plant tissue include the direct infection or co-cultivation of plant cell with Agrobacterium tumefaciens, Horsch et al., Science, 227:1229 (1985). Descriptions of Agrobacterium vector systems and methods for Agrobacterium-mediated gene transfer provided by Gruber et al. supra.

Alternatively, polynucleotides are introduced into plant cells or other plant tissues using a direct gene transfer method such as microprojectile-mediated delivery, DNA injection, electroporation and the like. More preferably polynucleotides are introduced into plant tissues using the microprojectile media delivery with the biolistic device. See, for example, Tomes et al., “Direct DNA transfer into intact plant cells via microprojectile bombardment” In: Gamborg and Phillips (Eds.) Plant Cell, Tissue and Organ Culture: Fundamental Methods, Springer Verlag, Berlin (1995).

In another embodiment of the current invention, expression constructs can be used for gene expression in callus culture for the purpose of expressing marker genes encoding peptides or polypeptides that allow identification of transformed plants. Here, a promoter that is operatively linked to a polynucleotide to be transcribed is transformed into plant cells and the transformed tissue is then placed on callus-inducing media. If the transformation is conducted with leaf discs, for example, callus will initiate along the cut edges. Once callus growth has initiated, callus cells can be transferred to callus shoot-inducing or callus root-inducing media. Gene expression will occur in the callus cells developing on the appropriate media: callus root-inducing promoters will be activated on callus root-inducing media, etc. Examples of such peptides or polypeptides useful as transformation markers include, but are not limited to barstar, glyphosate, chloramphenicol acetyltransferase (CAT), kanamycin, spectinomycin, streptomycin or other antibiotic resistance enzymes, green fluorescent protein (GFP), and β-glucuronidase (GUS), etc. Some of the exemplary promoters of the row titled “The predicted promoter sequence” will also be capable of sustaining expression in some tissues or organs after the initiation or completion of regeneration. Examples of these tissues or organs are somatic embryos, cotyledon, hypocotyl, epicotyl, leaf, stems, roots, flowers and seed.

Integration into the host cell genome also can be accomplished by methods known in the art, for example, by the homologous sequences or T-DNA discussed above or using the cre-lox system (A. C. Vergunst et al., Plant Mol. Biol. 38:393 (1998)).

### 11. Additional Uses for Promoters of the Invention

In yet another embodiment, the promoters of the present invention can be used to further understand developmental mechanisms. For example, promoters that are specifically induced during callus formation, somatic embryo formation, shoot formation or root formation can be used to explore the effects of overexpression, repression or ectopic expression of target genes, or for isolation of trans-acting factors.

The vectors of the invention can be used not only for expression of coding regions but may also be used in exon-trap cloning, or promoter trap procedures to detect differential gene expression in various tissues, K. Lindsey et al., 1993 “Tagging Genomic Sequences That Direct Transgene Expression by Activation of a Promoter Trap in Plants”, Transgenic Research 2:3347. D. Auch & Reth, et al., “Exon Trap Cloning: Using PCR to Rapidly Detect and Clone Exons from Genomic DNA Fragments”, Nucleic Acids Research, Vol. 18, No. 22, p. 674.

Entrapment vectors, first described for use in bacteria (Casadaban and Cohen, 1979, Proc. Nat. Aca. Sci. U.S.A., 76: 4530; Casadaban et al., 1980, J. Bacteriol., 143: 971) permit selection of insertional events that lie within coding sequences. Entrapment vectors can be introduced into pluripotent ES cells in culture and then passed into the germline via chimeras (Gossler et al., 1989, Science, 244: 463; Skarnes, 1990, Biotechnology, 8: 827). Promoter or gene trap vectors often contain a reporter gene, e.g., lacZ, lacking its own promoter and/or splice acceptor sequence upstream. That is, promoter gene traps contain a reporter gene with a splice site but no promoter. If the vector lands in a gene and is spliced into the gene product, then the reporter gene is expressed.

Recently, the isolation of preferentially-induced genes has been made possible with the use of sophisticated promoter traps (e.g. IVET) that are based on conditional auxotrophy complementation or drug resistance. In one IVET approach, various bacterial genome fragments are placed in front of a necessary metabolic gene coupled to a reporter gene. The DNA constructs are inserted into a bacterial strain otherwise lacking the metabolic gene, and the resulting bacteria are used to infect the host organism. Only bacteria expressing the metabolic gene survive in the host organism; consequently, inactive constructs can be eliminated by harvesting only bacteria that survive for some minimum period in the host. At the same time, constitutively active constructs can be eliminated by screening only bacteria that do not express the reporter gene under laboratory conditions. The bacteria selected by such a method contain constructs that are selectively induced only during infection of the host. The IVET approach can be modified for use in plants to identify genes induced in either the bacteria or the plant cells upon pathogen infection or root colonization. For information on IVET see the articles by Mahan et al. in Science 259:686-688 (1993), Mahan et al. in PNAS USA 92:669-673 (1995), Heithoff et al. in PNAS USA 94:934-939 (1997), and Wanget al. in PNAS USA. 93:10434 (1996).

11.1 Constitutive Transcription

Use of promoters and control elements providing constitutive transcription is desired for modulation of transcription in most cells of an organism under most environmental conditions. In a plant, for example, constitutive transcription is useful for modulating genes involved in defense, pest resistance, herbicide resistance, etc.

Constitutive up-regulation and transcription down-regulation is useful for these applications. For instance, genes, transcripts, and/or polypeptides that increase defense, pest and herbicide resistance may require constitutive up-regulation of transcription. In contrast, constitutive transcriptional down-regulation may be desired to inhibit those genes, transcripts, and/or polypeptides that lower defense, pest and herbicide resistance.

Typically, promoter or control elements that provide constitutive transcription produce transcription levels that are statistically similar in many tissues and environmental conditions observed.

Calculation of P-value from the different observed transcript levels is one means of determining whether a promoter or control element is providing constitutive up-regulation. P-value is the probability that the difference of transcript levels is not statistically significant. The higher the P-value, the more likely the difference of transcript levels is not significant. One formula used to calculate P-value is as follows:

$$\int ϕ ( x ) d x , integrated from a to ∞ , \\ where ϕ ( x ) is a normal distribution ;$$

$$where a = \frac{\vert Sx - μ \vert}{σ ( all Samples except Sx )} ;$$

$$where Sx = the intensity of the sample of interest$$

$$\begin{matrix}where μ = is the average of the intensities of all \\ samples except Sx , \\ = \frac{( Σ S 1 … Sn ) - Sx}{n - 1}\end{matrix}$$

where σ(S1 . . . S11, not including Sx)=the standard deviation of all sample intensities except Sx.

The P-value from the formula ranges from 1.0 to 0.0.

Usually, each P-value of the transcript levels observed in a majority of cells, tissues, or organs under various environmental conditions produced by the promoter or control element is greater than 10⁻⁸; more usually, greater than 10⁻⁷; even more usually, greater than 10⁻⁶; even more usually, greater than 10⁻⁵ or 10⁻⁴.

For up-regulation of transcription, promoter and control elements produce transcript levels that are above background of the assay.

11.2 Stress Induced Preferential Transcription

Promoters and control elements providing modulation of transcription under oxidative, drought, oxygen, wound, and methyl jasmonate stress are particularly useful for producing host cells or organisms that are more resistant to biotic and abiotic stresses. In a plant, for example, modulation of genes, transcripts, and/or polypeptides in response to oxidative stress can protect cells against damage caused by oxidative agents, such as hydrogen peroxide and other free radicals.

Drought induction of genes, transcripts, and/or polypeptides are useful to increase the viability of a plant, for example, when water is a limiting factor. In contrast, genes, transcripts, and/or polypeptides induced during oxygen stress can help the flood tolerance of a plant.

The promoters and control elements of the present invention can modulate stresses similar to those described in, for example, stress conditions are VuPLD1 (drought stress; Cowpea; see Pham-Thi et al. 1999. Plant molecular Biology. 1257-65), pyruvate decarboxylase (oxygen stress; rice; see Rivosal et al. 1997. Plant Physiol. 114(3): 1021-29), chromoplast specific carotenoid gene (oxidative stress; capsicum; see Bouvier et al. 1998. Journal of Biological Chemistry 273: 30651-59).

Promoters and control elements providing preferential transcription during wounding or induced by methyl jasmonate can produce a defense response in host cells or organisms. In a plant, for example, preferential modulation of genes, transcripts, and/or polypeptides under such conditions is useful to induce a defense response to mechanical wounding, pest or pathogen attack or treatment with certain chemicals.

Promoters and control elements of the present invention also can trigger a response similar to those described for cf9 (viral pathogen; tomato; see O'Donnell et al. 1998. The Plant journal: for cell and molecular biology 14(1): 137-42), hepatocyte growth factor activator inhibitor type 1 (HAI-1), which enhances tissue regeneration (tissue injury; human; Koono et al. 1999. Journal of Histochemistry and Cytochemistry 47: 673-82), copper amine oxidase (CuAO), induced during ontogenesis and wound healing (wounding; chick-pea; Rea et al. 1998. FEBS Letters 437: 177-82), proteinase inhibitor II (wounding; potato; see Pena-Cortes et al. 1988. Planta 174: 84-89), protease inhibitor II (methyl jasmonate; tomato; see Farmer and Ryan. 1990. Proc Natl Acad Sci USA 87: 7713-7716), two vegetative storage protein genes VspA and VspB (wounding, jasmonic acid, and water deficit; soybean; see Mason and Mullet. 1990. Plant Cell 2: 569-579).

Up-regulation and transcription down-regulation are useful for these applications. For instance, genes, transcripts, and/or polypeptides that increase oxidative, flood, or drought tolerance may require up-regulation of transcription. In contrast, transcriptional down-regulation may be desired to inhibit those genes, transcripts, and/or polypeptides that lower such tolerance.

Typically, promoter or control elements, which provide preferential transcription in wounding or under methyl jasmonate induction, produce transcript levels that are statistically significant as compared to cell types, organs or tissues under other conditions.

For preferential up-regulation of transcription, promoter and control elements produce transcript levels that are above background of the assay.

11.3 Light Induced Preferential Transcription

Promoters and control elements providing preferential transcription when induced by light exposure can be utilized to modulate growth, metabolism, and development; to increase drought tolerance; and decrease damage from light stress for host cells or organisms. In a plant, for example, modulation of genes, transcripts, and/or polypeptides in response to light is useful
(1) to increase the photosynthetic rate;
(2) to increase storage of certain molecules in leaves or green parts only, e.g., silage with high protein or starch content;
(3) to modulate production of exogenous compositions in green tissue, e.g., certain feed enzymes;
(4) to induce growth or development, such as fruit development and maturity, during extended exposure to light;
(5) to modulate guard cells to control the size of stomata in leaves to prevent water loss, or
(6) to induce accumulation of beta-carotene to help plants cope with light induced stress.
   The promoters and control elements of the present invention also can trigger responses similar to those described in: abscisic acid insensitive3 (ABI3) (dark-grown Arabidopsis seedlings, see Rohde et al. 2000. The Plant Cell 12: 35-52), asparagine synthetase (pea root nodules, see Tsai, F. Y.; Coruzzi, G. M. 1990. EMBO J 9: 323-32), mdm2 gene (human tumor; see Saucedo et al. 1998. Cell Growth Differ 9: 119-30).

Up-regulation and transcription down-regulation are useful for these applications. For instance, genes, transcripts, and/or polypeptides that increase drought or light tolerance may require up-regulation of transcription. In contrast, transcriptional down-regulation may be desired to inhibit those genes, transcripts, and/or polypeptides that lower such tolerance.

Typically, promoter or control elements, which provide preferential transcription in cells, tissues or organs exposed to light, produce transcript levels that are statistically significant as compared to cells, tissues, or organs under decreased light exposure (intensity or length of time).

For preferential up-regulation of transcription, promoter and control elements produce transcript levels that are above background of the assay.

11.4 Dark Induced Preferential Transcription

Promoters and control elements providing preferential transcription when induced by dark or decreased light intensity or decreased light exposure time can be utilized to time growth, metabolism, and development, to modulate photosynthesis capabilities for host cells or organisms. In a plant, for example, modulation of genes, transcripts, and/or polypeptides in response to dark is useful, for example,
(1) to induce growth or development, such as fruit development and maturity, despite lack of light;
(2) to modulate genes, transcripts, and/or polypeptide active at night or on cloudy days; or
(3) to preserve the plastid ultra structure present at the onset of darkness.
   The present promoters and control elements can also trigger response similar to those described in the section above.

Up-regulation and transcription down-regulation is useful for these applications. For instance, genes, transcripts, and/or polypeptides that increase growth and development may require up-regulation of transcription. In contrast, transcriptional down-regulation may be desired to inhibit those genes, transcripts, and/or polypeptides that modulate photosynthesis capabilities.

Typically, promoter or control elements, which provide preferential transcription under exposure to dark or decrease light intensity or decrease exposure time, produce transcript levels that are statistically significant.

For preferential up-regulation of transcription, promoter and control elements produce transcript levels that are above background of the assay.

11.5 Leaf Preferential Transcription

Promoters and control elements providing preferential transcription in a leaf can modulate growth, metabolism, and development or modulate energy and nutrient utilization in host cells or organisms. In a plant, for example, preferential modulation of genes, transcripts, and/or polypeptide in a leaf, is useful, for example,
(1) to modulate leaf size, shape, and development;
(2) to modulate the number of leaves; or
(3) to modulate energy or nutrient usage in relation to other organs and tissues

Up-regulation and transcription down-regulation is useful for these applications. For instance, genes, transcripts, and/or polypeptides that increase growth, for example, may require up-regulation of transcription. In contrast, transcriptional down-regulation may be desired to inhibit energy usage in a leaf to be directed to the fruit instead, for instance.

Typically, promoter or control elements, which provide preferential transcription in the cells, tissues, or organs of a leaf, produce transcript levels that are statistically significant as compared to other cells, organs or tissues.

For preferential up-regulation of transcription, promoter and control elements produce transcript levels that are above background of the assay.

11.6 Root Preferential Transcription

Promoters and control elements providing preferential transcription in a root can modulate growth, metabolism, development, nutrient uptake, nitrogen fixation, or modulate energy and nutrient utilization in host cells or organisms. In a plant, for example, preferential modulation of genes, transcripts, and/or in a leaf, is useful
(1) to modulate root size, shape, and development;
(2) to modulate the number of roots, or root hairs;
(3) to modulate mineral, fertilizer, or water uptake;
(4) to modulate transport of nutrients; or
(4) to modulate energy or nutrient usage in relation to other organs and tissues.

Up-regulation and transcription down-regulation is useful for these applications. For instance, genes, transcripts, and/or polypeptides that increase growth, for example, may require up-regulation of transcription. In contrast, transcriptional down-regulation may be desired to inhibit nutrient usage in a root to be directed to the leaf instead, for instance.

Typically, promoter or control elements, which provide preferential transcription in cells, tissues, or organs of a root, produce transcript levels that are statistically significant as compared to other cells, organs or tissues.

For preferential up-regulation of transcription, promoter and control elements produce transcript levels that are above background of the assay.

11.7 Stem/Shoot Preferential Transcription

Promoters and control elements providing preferential transcription in a stem or shoot can modulate growth, metabolism, and development or modulate energy and nutrient utilization in host cells or organisms. In a plant, for example, preferential modulation of genes, transcripts, and/or polypeptide in a stem or shoot, is useful, for example,
(1) to modulate stem/shoot size, shape, and development; or
(2) to modulate energy or nutrient usage in relation to other organs and tissues

Up-regulation and transcription down-regulation is useful for these applications. For instance, genes, transcripts, and/or polypeptides that increase growth, for example, may require up-regulation of transcription. In contrast, transcriptional down-regulation may be desired to inhibit energy usage in a stem/shoot to be directed to the fruit instead, for instance.

Typically, promoter or control elements, which provide preferential transcription in the cells, tissues, or organs of a stem or shoot, produce transcript levels that are statistically significant as compared to other cells, organs or tissues.

For preferential up-regulation of transcription, promoter and control elements produce transcript levels that are above background of the assay.

11.8 Fruit and Seed Preferential Transcription

Promoters and control elements providing preferential transcription in a silique or fruit can time growth, development, or maturity; or modulate fertility; or modulate energy and nutrient utilization in host cells or organisms. In a plant, for example, preferential modulation of genes, transcripts, and/or polypeptides in a fruit, is useful
(1) to modulate fruit size, shape, development, and maturity;
(2) to modulate the number of fruit or seeds;
(3) to modulate seed shattering;
(4) to modulate components of seeds, such as, storage molecules, starch, protein, oil, vitamins, anti-nutritional components, such as phytic acid;
(5) to modulate seed and/or seedling vigor or viability;
(6) to incorporate exogenous compositions into a seed, such as lysine rich proteins;
(7) to permit similar fruit maturity timing for early and late blooming flowers; or
(8) to modulate energy or nutrient usage in relation to other organs and tissues.

Up-regulation and transcription down-regulation is useful for these applications. For instance, genes, transcripts, and/or polypeptides that increase growth, for example, may require up-regulation of transcription. In contrast, transcriptional down-regulation may be desired to inhibit late fruit maturity, for instance.

Typically, promoter or control elements, which provide preferential transcription in the cells, tissues, or organs of siliques or fruits, produce transcript levels that are statistically significant as compared to other cells, organs or tissues.

For preferential up-regulation of transcription, promoter and control elements produce transcript levels that are above background of the assay.

11.9 Callus Preferential Transcription

Promoters and control elements providing preferential transcription in a callus can be useful to modulating transcription in dedifferentiated host cells. In a plant transformation, for example, preferential modulation of genes, transcripts, in callus is useful to modulate transcription of a marker gene, which can facilitate selection of cells that are transformed with exogenous polynucleotides.

Up-regulation and transcription down-regulation is useful for these applications. For instance, genes, transcripts, and/or polypeptides that increase marker gene detectability, for example, may require up-regulation of transcription. In contrast, transcriptional down-regulation may be desired to increase the ability of the calluses to later differentiate, for instance.

Typically, promoter or control elements, which provide preferential transcription in callus, produce transcript levels that are statistically significant as compared to other cell types, tissues, or organs. Calculation of P-value from the different observed transcript levels is one means of determining whether a promoter or control element is providing such preferential transcription.

Usually, each P-value of the transcript levels observed in callus as compared to, at least one other cell type, tissue or organ, is less than 10⁻⁴; more usually, less than 10⁻⁵; even more usually, less than 10⁻⁶; even more usually, less than 10⁻⁷ or 10⁻⁸.

For preferential up-regulation of transcription, promoter and control elements produce transcript levels that are above background of the assay.

11.10 Flower Specific Transcription

Promoters and control elements providing preferential transcription in flowers can modulate pigmentation; or modulate fertility in host cells or organisms. In a plant, for example, preferential modulation of genes, transcripts, and/or polypeptides in a flower, is useful,
(1) to modulate petal color; or
(2) to modulate the fertility of pistil and/or stamen.

Up-regulation and transcription down-regulation is useful for these applications. For instance, genes, transcripts, and/or polypeptides that increase pigmentation, for example, may require up-regulation of transcription. In contrast, transcriptional down-regulation may be desired to inhibit fertility, for instance.

Typically, promoter or control elements, which provide preferential transcription in flowers, produce transcript levels that are statistically significant as compared to other cells, organs or tissues.

For preferential up-regulation of transcription, promoter and control elements produce transcript levels that are above background of the assay.

11.11 Immature Bud and Inflorescence Preferential Transcription

Promoters and control elements providing preferential transcription in a immature bud or inflorescence can time growth, development, or maturity; or modulate fertility or viability in host cells or organisms. In a plant, for example, preferential modulation of genes, transcripts, and/or polypeptide in a fruit, is useful,
(1) to modulate embryo development, size, and maturity;
(2) to modulate endosperm development, size, and composition;
(3) to modulate the number of seeds and fruits; or
(4) to modulate seed development and viability.

Up-regulation and transcription down-regulation is useful for these applications. For instance, genes, transcripts, and/or polypeptides that increase growth, for example, may require up-regulation of transcription. In contrast, transcriptional down-regulation may be desired to decrease endosperm size, for instance.

Typically, promoter or control elements, which provide preferential transcription in immature buds and inflorescences, produce transcript levels that are statistically significant as compared to other cell types, organs or tissues.

For preferential up-regulation of transcription, promoter and control elements produce transcript levels that are above background of the assay.

11.12 Senescence Preferential Transcription

Promoters and control elements providing preferential transcription during senescence can be used to modulate cell degeneration, nutrient mobilization, and scavenging of free radicals in host cells or organisms. Other types of responses that can be modulated include, for example, senescence associated genes (SAG) that encode enzymes thought to be involved in cell degeneration and nutrient mobilization (Arabidopsis; see Hensel et al. 1993. Plant Cell 5: 553-64), and the CP-2/cathepsin L gene (rat; Kim and Wright. 1997. Biol Reprod 57: 1467-77), both induced during senescence.

In a plant, for example, preferential modulation of genes, transcripts, and/or polypeptides during senescencing is useful to modulate fruit ripening.

Up-regulation and transcription down-regulation is useful for these applications. For instance, genes, transcripts, and/or polypeptides that increase scavenging of free radicals, for example, may require up-regulation of transcription. In contrast, transcriptional down-regulation may be desired to inhibit cell degeneration, for instance.

Typically, promoter or control elements, which provide preferential transcription in cells, tissues, or organs during senescence, produce transcript levels that are statistically significant as compared to other conditions.

For preferential up-regulation of transcription, promoter and control elements produce transcript levels that are above background of the assay.

11.13 Germination Preferential Transcription

Promoters and control elements providing preferential transcription in a germinating seed can time growth, development, or maturity; or modulate viability in host cells or organisms. In a plant, for example, preferential modulation of genes, transcripts, and/or polypeptide in a germinating seed, is useful,
(1) to modulate the emergence of they hypocotyls, cotyledons and radical; or
(2) to modulate shoot and primary root growth and development;

Up-regulation and transcription down-regulation is useful for these applications. For instance, genes, transcripts, and/or polypeptides that increase growth, for example, may require up-regulation of transcription. In contrast, transcriptional down-regulation may be desired to decrease endosperm size, for instance.

Typically, promoter or control elements, which provide preferential transcription in a germinating seed, produce transcript levels that are statistically significant as compared to other cell types, organs or tissues.

For preferential up-regulation of transcription, promoter and control elements produce transcript levels that are above background of the assay.

### 12. GFP Experimental Procedures and Results

12.1 Procedures

The polynucleotide sequences of the present invention were tested for promoter activity using Green Fluorescent Protein (GFP) assays in the following manner.

Approximately 1-2 kb of genomic sequence occurring immediately upstream of the ATG translational start site of the gene of interest was isolated using appropriate primers tailed with BstXI restriction sites. Standard PCR reactions using these primers and genomic DNA were conducted. The resulting product was isolated, cleaved with BstXI and cloned into the BstXI site of an appropriate vector, such as pNewBin4-HAP1-GFP (see FIG. 1).

Transformation

The following procedure was used for transformation of plants

### 1. Stratification of WS-2 Seed.

Add 0.5 ml WS-2 (CS2360) seed to 50 ml of 0.2% Phytagar in a 50 ml Corning tube and vortex until seeds and Phytagar form a homogenous mixture.
Cover tube with foil and stratify at 4° C. for 3 days.

### 2. Preparation of Seed Mixture.

Obtain stratified seed from cooler.
Add seed mixture to a 1000 ml beaker.
Add an additional 950 ml of 0.2% Phytagar and mix to homogenize.

### 3. Preparation of Soil Mixture.

Mix 24 L SunshineMix #5 soil with 16 L Therm-O-Rock vermiculite in cement mixer to make a 60:40 soil mixture.
Amend soil mixture by adding 2 Tbsp Marathon and 3 Tbsp Osmocote and mix contents thoroughly.
Add 1 Tbsp Peters fertilizer to 3 gallons of water and add to soil mixture and mix thoroughly.
Fill 4-inch pots with soil mixture and round the surface to create a slight dome.
Cover pots with 8-inch squares of nylon netting and fasten using rubber bands.
Place 14 4-inch pots into each no-hole utility flat.

### 4. Planting.

Using a 60 ml syringe, aspirate 35 ml of the seed mixture.
Exude 25 drops of the seed mixture onto each pot.
Repeat until all pots have been seeded.
Place flats on greenhouse bench, cover flat with clear propagation domes, place 55% shade cloth on top of flats and subirrigate by adding 1 inch of water to bottom of each flat.

### 5. Plant Maintenance.

3 to 4 days after planting, remove clear lids and shade cloth.
Subirrigate flats with water as needed.
After 7-10 days, thin pots to 20 plants per pot using forceps.
After 2 weeks, subirrigate all plants with Peters fertilizer at a rate of 1 Tsp per gallon water.
When bolts are about 5-10 cm long, clip them between the first node and the base of stem to induce secondary bolts.
6 to 7 days after clipping, perform dipping infiltration.

### 6. Preparation of Agrobacterium.

Add 150 ml fresh YEB to 250 ml centrifuge bottles and cap each with a foam plug (Identi-Plug).
Autoclave for 40 min at 121° C.
After cooling to room temperature, uncap and add 0.1 ml each of carbenicillin, spectinomycin and rifampicin stock solutions to each culture vessel.
Obtain Agrobacterium starter block (96-well block with Agrobacterium cultures grown to an OD₆₀₀ of approximately 1.0) and inoculate one culture vessel per construct by transferring 1 ml from appropriate well in the starter block.
Cap culture vessels and place on Lab-Line incubator shaker set at 27° C. and 250 RPM.
Remove after Agrobacterium cultures reach an OD₆₀₀ of approximately 1.0 (about 24 hours), cap culture vessels with plastic caps, place in Sorvall SLA 1500 rotor and centrifuge at 8000 RPM for 8 min at 4° C.
Pour out supernatant and put bottles on ice until ready to use.
Add 200 ml Infiltration Media (IM) to each bottle, resuspend Agrobacterium pellets and store on ice.

### 7. Dipping Infiltration.

Pour resuspended Agrobacterium into 16 oz polypropylene containers.
Invert 4-inch pots and submerge the aerial portion of the plants into the Agrobacterium suspension and let stand for 5 min.
Pour out Agrobacterium suspension into waste bucket while keeping polypropylene container in place and return the plants to the upright position.
Place 10 covered pots per flat.
Fill each flat with 1-inch of water and cover with shade cloth.
Keep covered for 24 hr and then remove shade cloth and polypropylene containers.
Resume normal plant maintenance.
When plants have finished flowering cover each pot with a ciber plant sleeve.
After plants are completely dry, collect seed and place into 2.0 ml micro tubes and store in 100-place cryogenic boxes.

### Recipes:

### 0.2% Phytagar

2 g Phytagar

1 L nanopure water
Shake until Phytagar suspended
Autoclave 20 min

### YEB (for 1 L)

5 g extract of meat

5 g Bacto peptone

1 g yeast extract

5 g sucrose

0.24 g magnesium sulfate
While stirring, add ingredients, in order, to 900 ml nanopure water
When dissolved, adjust pH to 7.2
Fill to 1 L with nanopure water
Autoclave 35 min

### Infiltration Medium (IM) (for 1 L)

2.2 g MS salts

50 g sucrose

5 ul BAP solution (stock is 2 mg/ml)
While stirring, add ingredients in order listed to 900 ml nanopure water
When dissolved, adjust pH to 5.8.
Volume up to 1 L with nanopure water.
Add 0.02% Silwet L-77 just prior to resuspending Agrobacterium

High Throughput Screening—T1 Generation

1. Soil Preparation. Wear gloves at all times.
In a large container, mix 60% autoclaved SunshineMix #5 with 40% vermiculite.
Add 2.5 Tbsp of Osmocote, and 2.5 Tbsp of 1% granular Marathon per 25 L of soil.
Mix thoroughly.

### 2. Fill Com-Packs With Soil.

Loosely fill D601 Com-Packs level to the rim with the prepared soil.
Place filled pot into utility flat with holes, within a no-hole utility flat.
Repeat as necessary for planting. One flat set should contain 6 pots.

### 3. Saturate Soil.

Evenly water all pots until the soil is saturated and water is collecting in the bottom of the flats.
After the soil is completely saturated, dump out the excess water.

### 4. Plant the Seed.

### 5. Stratify the Seeds.

After sowing the seed for all the flats, place them into a dark 4° C. cooler.
Keep the flats in the cooler for 2 nights for WS seed. Other ecotypes may take longer. This cold treatment will help promote uniform germination of the seed.
   6. Remove Flats From Cooler and Cover With Shade Cloth. (Shade cloth is only needed in the greenhouse)
After the appropriate time, remove the flats from the cooler and place onto growth racks or benches.
Cover the entire set of flats with 55% shade cloth. The cloth is necessary to cut down the light intensity during the delicate germination period.
The cloth and domes should remain on the flats until the cotyledons have fully expanded. This usually takes about 4-5 days under standard greenhouse conditions.

### 7. Remove 55% Shade Cloth and Propagation Domes.

After the cotyledons have fully expanded, remove both the 55% shade cloth and propagation domes.
   8. Spray Plants With Finale Mixture. Wear gloves and protective clothing at all times.
Prepare working Finale mixture by mixing 3 ml concentrated Finale in 48 oz of water in the Poly-TEK sprayer.
Completely and evenly spray plants with a fine mist of the Finale mixture.
Repeat Finale spraying every 3-4 days until only transformants remain. (Approximately 3 applications are necessary.)
When satisfied that only transformants remain, discontinue Finale spraying.

### 9. Weed Out Excess Transformants.

Weed out excess transformants such that a maximum number of five plants per pot exist evenly spaced throughout the pot.

12.2 GFP Assay

Tissues are dissected by eye or under magnification using INOX 5 grade forceps and placed on a slide with water and coversliped. An attempt is made to record images of observed expression patterns at earliest and latest stages of development of tissues listed below. Specific tissues will be preceded with High (H), Medium (M), Low (L) designations.

| | |
|---|---|
| [symbol] Flower | [symbol] pedicel [symbol] receptacle [symbol] nectary [symbol] sepal [symbol] petal [symbol] filament [symbol] anther [symbol] pollen [symbol] carpel [symbol] style [symbol] papillae [symbol] vascular |
| | [symbol] epidermis [symbol] stomata [symbol] trichome |
| [symbol] Silique | [symbol] stigma [symbol] style [symbol] carpel [symbol] septum [symbol] placentae [symbol] transmitting tissue [symbol] vascular [symbol] epidermis [symbol] stomata |
| | [symbol] abscission zone [symbol] ovule |
| [symbol] Ovule | Pre-fertilization: [symbol] inner integument [symbol] outer integument [symbol] embryo sac [symbol] funiculus [symbol] chalaza [symbol] micropyle |
| | [symbol] gametophyte |
| [symbol] Embryo | Post-fertilization: [symbol] zygote [symbol] inner integument [symbol] outer integument [symbol] seed coat [symbol] primordia [symbol] chalaza |
| | [symbol] micropyle [symbol] early endosperm [symbol] mature endosperm [symbol] embryo |
| | [symbol] suspensor [symbol] preglobular [symbol] globular [symbol] heart [symbol] torpedo [symbol] late [symbol] mature [symbol] provascular [symbol] hypophysis [symbol] radicle |
| | [symbol] cotyledons [symbol] hypocotyl |
| [symbol] Stem | [symbol] epidermis [symbol] cortex [symbol] vascular [symbol] xylem [symbol] phloem [symbol] pith [symbol] stomata [symbol] trichome |
| [symbol] Leaf | [symbol] petiole [symbol] mesophyll [symbol] vascular [symbol] epidermis [symbol] trichome [symbol] primordia [symbol] stomata [symbol] stipule [symbol] margin |

T1 Mature: These are the T1 plants resulting from independent transformation events. These are screened between stage 6.50-6.90 (means the plant is flowering and that 50-90% of the flowers that the plant will make have developed) which is 4-6 weeks of age. At this stage the mature plant possesses flowers, siliques at all stages of development, and fully expanded leaves. We do not generally differentiate between 6.50 and 6.90 in the report but rather just indicate 6.50. The plants are initially imaged under UV with a Leica Confocal microscope. This allows examination of the plants on a global level. If expression is present, they are imaged using scanning laser confocal microscopy.

T2 Seedling: Progeny are collected from the T1 plants giving the same expression pattern and the progeny (T2) are sterilized and plated on agar-solidified medium containing M&S salts. In the event that there was no expression in the T1 plants, T2 seeds are planted from all lines. The seedlings are grown in Percival incubators under continuous light at 22° C. for 10-12 days. Cotyledons, roots, hypocotyls, petioles, leaves, and the shoot meristem region of individual seedlings were screened until two seedlings were observed to have the same pattern. Generally found the same expression pattern was found in the first two seedlings. However, up to 6 seedlings were screened before “no expression pattern” was recorded. All constructs are screened as T2 seedlings even if they did not have an expression pattern in the T1 generation.

T2 Mature: The T2 mature plants were screened in a similar manner to the T1 plants. The T2 seeds were planted in the greenhouse, exposed to selection and at least one plant screened to confirm the T1 expression pattern. In instances where there were any subtle changes in expression, multiple plants were examined and the changes noted in the tables.

T3 Seedling: This was done similar to the T2 seedlings except that only the plants for which we are trying to confirm the pattern are planted.

12.3 Image Data:

Images are collected by scanning laser confocal microscopy. Scanned images are taken as 2-D optical sections or 3-D images generated by stacking the 2-D optical sections collected in series. All scanned images are saved as TIFF files by imaging software, edited in Adobe Photoshop, and labeled in Powerpoint specifying organ and specific expressing tissues.

### Instrumentation:

### Microscope

Inverted Leica DM IRB
   Fluorescence filter blocks:
Blue excitation BP 450-490; long pass emission LP 515.
Green excitation BP 515-560; long pass emission LP 590

### Objectives

HC PL FLUOTAR 5×/0.5
HCPL APO 10×/0.4 IMM water/glycerol/oil
HCPL APO 20×/0.7 IMM water/glycerol/oil
HCXL APO 63×/1.2 IMM water/glycerol/oil

### Leica TCS SP2 Confocal Scanner

Spectral range of detector optics 400-850 nm.
Variable computer controlled pinhole diameter.
Optical zoom 1-32×.
   Four simultaneous detectors:
Three channels for collection of fluorescence or reflected light.
One channel for transmitted light detector.
   Laser sources:
Blue Ar 458/5 mW, 476 nm/5 mW, 488 nm/20 mW, 514 nm/20 mW.
Green HeNe 543 nm/1.2 mW
Red HeNe 633 nm/10 mW

12.4 Results

The section in Table 1 entitled “The spatial expression of the promoter-marker-vector” presents the results of the GFP assays as reported by their corresponding cDNA ID number, construct number and line number. Table 1 includes various information about each promoter or promoter control element of the invention including the nucleotid sequence, the spatial expression promoted by each promoter, and the corresponding results from different expression experiments. GFP data gives the location of expression that is visible under the imaging parameters. Table 2 summarizes the results of the spatial expression results for the promoters.

TABLE 1
Promoter Sequences and Related Information

Promoter YP0396
Modulates the gene: PAR-related protein
The GenBank description of the gene: : NM_124618 Arabidopsis thaliana photoassimilate-
responsive protein PAR-related protein (At5g52390) mRNA, complete cds
gi|30696178|ref|NM_124618.2|[30696178]
The promoter sequence (SEQ ID NO: 1):
5′ctaagtaaaataagataaaacatgttatttgaatttgaatatcgtgggatgcgtatttcggtatttgat
taaaggtctggaaaccggagctcctataacccgaataaaaatgcataacatgttcttccccaacgaggcga
gcgggtcagggcactagggtcattgcaggcagctcataaagtcatgatcatctaggagatcaaattgtatg
tcggccttctcaaaattacctctaagaatctcaaacccaatcatagaacctctaaaaagacaaagtcgtcg
ctttagaatgggttcggtttttggaaccatatttcacgtcaatttaatgtttagtataatttctgaacaac
agaattttggatttatttgcacgtatacaaatatctaattaataaggacgactcgtgactatccttacatt
aagtttcactgtcgaaataacatagtacaatacttgtcgttaatttccacgtctcaagtctataccgtcat
ttacggagaaagaacatctctgtttttcatccaaactactattctcactttgtctatatatttaaaattaa
gtaaaaaagactcaatagtccaataaaatgatgaccaaatgagaagatggttttgtgccagattttaggaa
aagtgagtcaaggtttcacatctcaaatttgactgcataatcttcgccattaacaacggcattatatatgt
caagccaattttccatgttgcgtacttttctattgaggtgaaaatatgggtttgttgattaatcaaagagt
ttgcctaactaatataactacgactttttcagtgaccattccatgtaaactctgcttagtgtttcatttgt
caacaatattgtcgttactcattaaatcaaggaaaaatatacaattgtataattttcttatattttaaaat
taattttga 3′

(SEQ ID NO: 2)

ccaaaagaacatctttccttcgaattttctttcattaacatttcttttacttgtctccttgtgtcttcact
tcacatcacaacATG
The promoter was cloned from the organism: Arabidopsis thaliana, Columbia ecotype

Alternative nucleotides:

| Predicted Position (bp) | Mismatch | Predicted/Experimental |
|---|---|---|
| 1-1000 | None | Identities = 1000/1000 (100%) |

The promoter was cloned in the vector: pNewbin4-HAP1-GFP
When cloned into the vector the promoter was operably linked to a marker, which was
the type: GFP-ER
Promoter-marker vector was tested in: Arabidopsis thaliana, WS ecotype
Generation screened: XT1 Mature XT2 Seedling T2 Mature T3 Seedling
The spatial expression of the promoter-marker vector was found observed in and would
be useful in expression in any or all of the following:

| | |
|---|---|
| Flower | H sepal H petal H anther H style |
| Silique | H style H ovule |
| Ovule | H outer integument H outer integument L seed coat |
| Leaf | H vascular |
| Primary Root | H epidermis |

Observed expression pattern:
T1 mature: High GFP expression in the style, sepals, petals, and anthers in flowers.
Expressed in outer integuments of ovule primordia through developing seed stages and
in remnants of aborted ovules. High vasculature expression in leaf
T2 seedling: Medium to low root epidermal expression at root transition zone
decreasing toward root tip.
Specific to epidermal cells flanking lateral roots.

| | | |
|---|---|---|
| Misc. promoter information: Bidirectionality: Pass | Exons: Pass | Repeats: No |

The Ceres cDNA ID of the endogenous coding sequence to the promoter: 12646726
cDNA nucleotide sequence (SEQ ID NO: 3):
ACTACACCCAAAAGAACATCTTTCCTTCGAATTTTCTTTCAATTAACATTTCTTTTACTTGTCTC
CTTGTGTCTTCACTTCACATCACAACATGGCTTTGAAGACAGTTTTCGTAGCTTTTATGATTCT
CCTTGCCATCTATTCGCAAACGACGTTTGGGGACGATGTGAAGTGCGAGAATCTGGATGAAAA
CACGTGTGCCTTCGCGGTCTCGTCCACTGGAAAACGTTGCGTTTTGGAGAAGAGCATGAAGAG
GAGCGGGATCGAGGTGTACACATGTCGATCATCGGAGATAGAAGCTAACAAGGTCACAAACA
TTATTGAATCGGACGAGTGCATTAAAGCGTGTGGTCTAGACCGGAAAGCTTTAGGTATATCTT
CGGACGCATTGTTGGAATCTCAGTTCACACATAAACTCTGCTCGGTTAAATGCTTAAACCAAT
GTCCTAACGTAGTCGATCTCTACTTCAACCTTGCTGCTGGTGAAGGAGTGTATTTACCAAAGCT
ATGTGAATCACAAGAAGGGAAGTCAAGAAGAGCAATGTCGGAAATTAGGAGCTCGGGAATTG
CAATGGACACTCTTGCACCGGTTGGACCAGTCATGTTGGGCGAGATAGCACCTGAGCCGGCTA
CTTCAATGGACAACATGCCTTACGTGCCGGCACCTTCACCGTATTAATTAAGGCAAGGGAAAA
TGGAGAGGACACGTATGATATCATGAGTTTTCGACGAGAATAATTAAGAGATTTATGTTTAGT
TCGACGGTTTTAGTATTACATCGTTTATTGCGTCCTTATATATATGTACTTCATAAAAACACAC
CACGACACATTAAGAGATGGTGAAAGTAGGCTGCGTTCTGGTGTAACTTTTACACAAGTAACG
TCTTATAATATATATGATTCGAATAAAATGTTGAGTTTTGGTGAAAATATATAATATGTTTCTG
Coding sequence (SEQ ID NO: 4):
MALKTVFVAFMILLAIYSQTTFGDDVKCENLDENTCAFAVSSTGKRCVLEKSMKRSGIEVYTCRSS
EIEANKVTNIIESDECIKACGLDRKALGISSDALLESQFTHKLCSVKCLNQCPNVVDLYFNLAAGEG
VYLPKLCESQEGKSRRAMSEIRSSGIAMDTLAPVGPVMLGEIAPEPATSMDNMPYVPAPSPY*
Promoter YP0388
Modulates the gene: protein phosphatase 2C (PP2C), putative
The GenBank description of the gene: NM_125312 Arabidopsis thaliana protein
phosphatase 2C (PP2C), putative (At5g59220) mRNA, complete cds
gi|30697191|ref|NM_25312.2|[30697191]
The promoter sequence (SEQ ID NO: 5):
5′tatttgtagtgacatattctacaattatcacatttttctcttatgtttcgtagtcgcagatggtca
attttttctataataatttgtccttgaacacaccaaactttagaaacgatgatatataccgtattgtc
acgctcacaatgaaacaaacgcgatgaatcgtcatcaccagctaaaagcctaaaacaccatcttagtt
ttcactcagataaaaagattatttgtttccaacctttctattgaattgattagcagtgatgacgtaat
tagtgatagtttatagtaaaacaaatggaagtggtaataaatttacacaacaaaatatggtaagaatc
tataaaataagaggttaagagatctcatgttatattaaatgattgaaagaaaaacaaactattggttg
atttccatatgtaatagtaagttgtgatgaaagtgatgacgtaattagttgtatttatagtaaaacaa
attaaaatggtaaggtaaatttccacaacaaaacttggtaaaaatcttaaaaaaaaaaaaagaggttt
agagatcgcatgcgtgtcatcaaaggttctttttcactttaggtctgagtagtgttagactttgattg
gtgcacgtaagtgtttcgtatcgcgatttaggagaagtacgttttacacgtggacacaatcaacggtc
aagatttcgtcgtccagatagaggagcgatacgtcacgccattcaacaatctcctcttcttcattcct
tcattttgattttgagttttgatctgcccgttcaaaagtctcggtcatctgcccgtaaatataaagat
gattatatttatttatatcttctggtgaaagaagctaaTATAaagcttccatggctaatcttgtttaa
gcttctcttcttcttctctctcctgtgtctcgttcactagttttttttcgggggagagtgatggagtg
tgtttgttgaata 3′ cATG
The promoter was cloned from the organism: Arabidopsis thaliana, Columbia ecotype

Alternative nucleotides:

| Predicted Position (bp) | Mismatch | Predicted/Experimental |
|---|---|---|
| 1-1000 | None | Identities = 1000/1000 (100%) |

The promoter was cloned in the vector: pNewbin4-HAP1-GFP
When cloned into the vector the promoter was operably linked to a marker, which was
the type: GFP-ER
Promoter-marker vector was tested in: Arabidopsis thaliana, WS ecotype
Generation screened: XT1 Mature XT2 Seedling T2 Mature T3 Seedling
The spatial expression of the promoter-marker vector was found observed in and would
be useful in expression in any or all of the following:

| | |
|---|---|
| Flower | H filament H anther H stomata |
| Silique | H ovule |
| Ovule | Post-fertilization: H outer H seed coat H chalaza |
| Leaf | L vascular H stomata |
| Primary Root | H epidermis |

Observed expression pattern:
T1 mature: Very high GFP expression levels in stamens of developing flowers. Low
expression in vasculature of leaves and guard cells throughout plant. High expression
in outer integument of ovules and in seed coats. High incidence of aborted ovules.
T2 seedling: Low expression in root epidermal cells.

| | | |
|---|---|---|
| Misc. promoter information: Bidirectionality: Pass | Exons: Pass | Repeats: No |

Optional Promoter Fragments: 5′ UTR region at base pairs 880-987.
The Ceres cDNA ID of the endogenous coding sequence to the promoter: 13593066
cDNA nucleotide sequence (SEQ ID NO: 6):
AAAGCTTCCATGGCTAATCTTGTTTAAGCTTCTCTTCTTCTTCTCTCTCCTGTGTCTCGTTCACT
AGTTTTTTTTCGGGGGAGAGTGATGGAGTGTGTTTGTTGAATAGTTTTGACGATCACATGGCT
GAGATTTGTTACGAGAACGAGACTATGATGATTGAAACGACGGCGACGGTGGTGAAGAAGGC
AACGACGACAACGAGGAGACGAGAACGGAGCTCGTCTCAAGCAGCGAGAAGAAGGAGAATG
GAGATCCGGAGGTTTAAGTTTGTTTCCGGCGAACAAGAACCTGTCTTCGTCGACGGTGACTTA
CAGAGGCGGAGGAGAAGAGAATCCACCGTCGCAGCCTCCACCTCCACCGTGTTTTACGAAACG
GCGAAGGAAGTTGTCGTCCTATGCGAGTCTCTTAGTTCAACGGTTGTGGCATTGCCTGATCCT
GAAGCTTATCCTAAATACGGCGTCGCTTCAGTCTGTGGAAGAAGACGTGAAATGGAAGACGCC
GTCGCTGTGCATCCGTTTTTTTCCCGTCATCAGACGGAATATTCATCCACCGGATTTCACTATT
GCGGCGTTTACGATGGCCATGGCTGTTCCCATGTAGCGATGAAATGTAGAGAAAGACTACACG
AGCTAGTCCGTGAAGAGTTTGAAGCTGATGCTGACTGGGAAAAGTCAATGGCGCGTAGCTTCA
CGCGCATGGACATGGAGGTTGTTGCGTTGAACGCCGATGGTGCGGCAAAATGCCGGTGCGAG
CTTCAGAGGCCGGACTGCGACGCGGTGGGATCCACTGCGGTTGTGTCTGTCCTTACGCCGGAG
AAAATCATCGTGGCGAATTGCGGTGACTCACGTGCCGTTCTCTGTCGTAACGGCAAAGCCATT
GCTTTATCCTCCGATCATAAGCCAGACCGTCCGGACGAGCTAGACCGGATTCAAGCAGCGGGT
GGTCGTGTTATCTACTGGGATGGCCCACGTGTCCTTGGAGTACTTGCAATGTCACGAGCCATT
GGAGATAATTACTTGAAGCCGTATGTAATCAGCAGACCGGAGGTAACCGTGACGGACCGGGC
CAACGGAGACGATTTTCTTATTCTCGCAAGTGACGGTCTTTGGGACGTTGTTTCAAACGAAAC
TGCATGTAGCGTCGTTCGAATGTGTTTGAGAGGAAAAGTCAATGGTCAAGTATCATCATCACC
GGAAAGGGAAATGACAGGTGTCGGCGCCGGGAATGTGGTGGTTGGAGGAGGAGATTTGCCAG
ATAAAGCGTGTGAGGAGGCGTCGCTGTTGCTGACGAGGCTTGCGTTGGCTAGACAAAGTTCGG
ACAACGTAAGTGTTGTGGTGGTTGATCTACGACGAGACACGTAGTTGTATTTGTCTCTCTCGT
AATGTTTGTTGTTTTTTGTCCTGAGTCATCGACTTTTGGGCTTTTTCTTTTAACCTTTTTTGCTC
TTCGGTGTAAGACAACGAAGGGTTTTTAATTTAGCTTGACTATGGGTTATGTCAGTCACTGTGT
TGAATCGCGGTTTAGATCTACAAAGATTTTCACCAGTAGTGAAAATGGTAAAAAGCCGTGAAA
TGTGAAAGACTTGAGTTCAATTTAATTTTAAATTTAATAGAATCAGTTGATC
Coding sequence (SEQ ID NO: 7):
MAEICYENETMMIETTATVVKKATTTTRRRERSSSQAARRRRMEIRRFKFVSGEQEPVFVDGDLQ
RRRRRESTVAASTSTVFYETAKEVVVLCESLSSTVVALPDPEAYPKYGVASVCGRRREMEDAVAV
HPFFSRHQTEYSSTGFHYCGVYDGHGCSHVAMKCRERLHELVREEFEADADWEKSMARSFTRMD
MEVVALNADGAAKCRCELQRPDCDAVGSTAVVSVLTPEKIIVANCGDSRAVLCRNGKAIALSSDH
KPDRPDELDRIQAAGGRVIYWDGPRVLGVLAMSRATGDNYLKPYVISRPEVTVTDRANGDDFLILA
SDGLWDVVSNETACSVVRMCLRGKVNGQVSSSPEREMTGVGAGNVVVGGGDLPDKACEEASLL
LTRLALARQSSDNVSVVVVDLRRDT*
Promoter YP0385
Modulates the gene: Neoxanthin cleavage enzyme.
The GenBank description of the gene: NM_112304 Arabidopsis thaliana 9-cis-
epoxycarotenoid dioxygenase [neoxanthin cleavage enzyme](NC1)(NCED1), putative
(At3g14440) mRNA, complete cds gi|30683162|ref|NM_112304.2|[30683162].
The promoter sequence (SEQ ID NO: 8):
5′aaaartccaattattgtgttactctattcttctaaatttgaacactaatagactatgacatatgagtat
ataatgtgaagtcttaagatattttcatgtgggagatgaataggccaagttggagtctgcaaacaagaagc
tcttgagccacgacataagccaagttgatgaccgtaattaatgaaactaaatgtgtgtggttatatattag
ggacccatggccatatacacaatttttgtttctgtcgatagcatgcgtttatatatatttctaaaaaaact
aacatatttactggatttgagttcgaatattgacactaatataaactacgtaccaaactacatatgtttat
ctatatttgattgatcgaagaattctgaactgttttagaaaatttcaatacacttaacttcatcttacaac
ggtaaaagaaatcaccactagacaaacaatgcctcataatgtctcgaaccctcaaactcaagagtatacat
tttactagattagagaatttgatatcctcaagttgccaaagaattggaagcttttgttaccaaacttagaa
acagaagaagccacaaaaaaagacaaagggagttaaagattgaagtgatgcatttgtctaagtgtgaaagg
tctcaagtctcaactttgaaccataataacattactcacactccctttttttttctttttttttcccaaag
taccctttttaattccctctataacccactcactccattccctctttctgtcactgattcaacacgtggcc
acactgatgggatccacctttcctcttacccacctcccggttTATAtaaacccttcacaacacttcatcgc
tctcaaaccaactctctcttctctcttctctcctctcttctacaagaagaaaaaaaacagagcctttacac
atctcaaaatcgaacttactttaaccacc 3′-aATG
The promoter was cloned from the organism: Arabidopsis thaliana, Columbia ecotype

Alternative nucleotides:

| | | |
|---|---|---|
| Predicted Position (bp) | Mismatch | Predicted/Experimental |

| | | |
|---|---|---|
| 7 | PCR error or ecotype variant SNP | g/- |
| 28 | Read error | a/a corrected |
| 29 | PCR error or ecotype variant SNP | a/- |

The promoter was cloned in the vector: pNewbin4-HAP1-GFP
When cloned into the vector the promoter was operably linked to a marker, which was
the type: GFP-ER
Promoter-marker vector was tested in: Arabidopsis thaliana, WS ecotype
Generation screened: XT1 Mature XT2 Seedling T2 Mature T3 Seedling
The spatial expression of the promoter-marker vector was found observed in and would
be useful in expression in any or all of the following:

| | |
|---|---|
| Flower | L receptacle |
| Silique | L abscission zone |
| Primary Root | H epidermis |

Observed expression pattern of the promoter-marker vector was in:
T1 mature: Expression specific to abscission zone of mature flowers.
T2 seedling: Expression in root epidermal cells. Expression rapidly decreases from
root transition zone to mid root.

| | | |
|---|---|---|
| Misc. promoter information: Bidirectionality: Pass | Exons: Pass | Repeats: No |

Optional Promoter Fragments: 5′ UTR region at base pairs 880-999.
The Ceres cDNA ID of the endogenous coding sequence to the promoter: 12658348
cDNA nucleotide sequence (SEQ ID NO: 9):
AAACCAACTCTCTCTTCTCTCTTCTCTCCTCTCTTCTACAAGAAGAAAAAAAACAGAGCCTTTA
CACATCTCAAAATCGAACTTACTTTAACCACCAAATACTGATTGAACACACTTGAAAAATGGC
TTCTTTCACGGCAACGGCTGCGGTTTCTGGGAGATGGCTTGGTGGCAATCATACTCAGCCGCC
ATTATCGTCTTCTCAAAGCTCCGACTTGAGTTATTGTAGCTCCTTACCTATGGCCAGTCGTGTC
ACACGTAAGCTCAATGTTTCATCTGCGCTTCACACTCCTCCAGCTCTTCATTTCCCTAAGCAAT
CATCAAACTCTCCCGCCATTGTTGTTAAGCCCAAAGCCAAAGAATCCAACACTAAACAGATGA
ATTTGTTCCAGAGAGCGGCGGCGGCAGCGTTGGACGCGGCGGAGGGTTTCCTTGTCAGCCACG
AGAAGCTACACCCGCTTCCTAAAACGGCTGATCCTAGTGTTCAGATCGCCGGAAATTTTGCTC
CGGTGAATGAACAGCCCGTCCGGCGTAATCTTCCGGTGGTCGGAAAACTTCCCGATTCCATCA
AAGGAGTGTATGTGCGCAACGGAGCTAACCCACTTCACGAGCCGGTGACAGGTCACCACTTCT
TCGACGGAGACGGTATGGTTCACGCCGTCAAATTCGAACACGGTTCAGCTAGCTACGCTTGCC
GGTTTACTCAGACTAACCGGTTTGTTCAGGAACGTCAATTGGGTCGACCGGTTTTCCCCAAAG
CCATCGGTGAGCTTCACGGCCACACCGGTATTGCCCGACTCATGCTATTCTACGCCAGAGCTG
CAGCCGGTATAGTCGACCCGGCACACGGAACCGGTGTAGCTAACGCCGGTTTGGTCTATTTCA
ATGGCCGGTTATTGGCTATGTCGGAGGATGATTTACCTTACCAAGTTCAGATCACTCCCAATG
GAGATTTAAAAACCGTTGGTCGGTTCGATTTTGATGGACAATTAGAATCCACAATGATTGCCC
ACCCGAAAGTCGACCCGGAATCCGGTGAACTCTTCGCTTTAAGCTACGACGTCGTTTCAAAGC
CTTACCTAAAATACTTCCGATTCTCACCGGACGGAACTAAATCACCGGACGTCGAGATTCAGC
TTGATCAGCCAACGATGATGCACGATTTCGCGATTACAGAGAACTTCGTCGTCGTACCTGACC
AGCAAGTCGTTTTCAAGCTGCCGGAGATGATCCGCGGTGGGTCTCCGGTGGTTTACGACAAGA
ACAAGGTCGCAAGATTCGGGATTTTAGACAAATACGCCGAAGATTCATCGAACATTAAGTGGA
TTGATGCTCCAGATTGCTTCTGCTTCCATCTCTGGAACGCTTGGGAAGAGCCAGAAACAGATG
AAGTCGTCGTGATAGGGTCCTGTATGACTCCACCAGACTCAATTTTCAACGAGTCTGACGAGA
ATCTCAAGAGTGTCCTGTCTGAAATCCGCCTGAATCTCAAAACCGGTGAATCAACTCGCCGTC
CGATCATCTCCAACGAAGATCAACAAGTCAACCTCGAAGCAGGGATGGTCAACAGAAACATG
CTCGGCCGTAAAACCAAATTCGCTTACTTGGCTTTAGCCGAGCCGTGGCCTAAAGTCTCAGGA
TTCGCTAAAGTTGATCTCACTACTGGAGAAGTTAAGAAACATCTTTACGGCGATAACCGTTAC
GGAGGAGAGCCTCTGTTTCTCCCCGGAGAAGGAGGAGAGGAAGACGAAGGATACATCCTCTG
TTTCGTTCACGACGAGAAGACATGGAAATCGGAGTTACAGATAGTTAACGCCGTTAGCTTAGA
GGTTGAAGCAACGGTTAAACTTCCGTCAAGGGTTCCGTACGGATTTCACGGTACATTCATCGG
AGCCGATGATTTGGCGAAGCAGGTCGTGTGAGTTCTTATGTGTAAATACGCACAAAATACATA
TACGTGATGAAGAAGCTTCTAGAAGGAAAAGAGAGAGCGAGATTTACCAGTGGGATGCTCTG
CATATACGTCCCCGGAATCTGCTCCTCTGTTTTTTTTTTTTTGCTCTGTTTCTTGTTTGTTGTTTC
TTTTGGGGTGCGGTTTGCTAGTTCCCTTTTTTTTGGGGTCAATCTAGAAATCTGAAAGATTTTG
AGGGACCAGCTTGTAGCTTTTGGGCTGTAGGGTAGCCTAGCCGTTCGAGCTCAGCTGGTTTCT
GTTATTCTTTCACTTATTGTTCATCGTAATGAGAAGTATATAAAATATTAAACAACAAAGATAT
GTTTGTATATGTGCATGAATTAAGGAACATTTTTTTT
Coding sequence (SEQ ID NO: 10):
MASFTATAAVSGRWLGGNHTQPPLSSSQSSDLSYCSSLPMASRVTRKLNVSSALHTPPALHFPKQS
SNSPAIVVKPKAKESNTKQMNLFQRAAAAALDAAEGFLVSHEKLHPLPKTADPSVQIAGNFAPVN
EQPVRRNLPVVGKLPDSIKGVYVRNGANPLHEPVTGHHFFDGDGMVHAVKFEHGSASYACRFTQ
TNRFVQERQLGRPVFPKAIGELHGHTGIARLMLFYARAAAGIVDPAHGTGVANAGLVYFNGRLLA
MSEDDLPYQVQITPNGDLKTVGRFDFDGQLESTMIAHPKVDPESGELFALSYDVVSKPYLKYFRFS
PDGTKSPDVEIQLDQPTMMHDFAITENFVVVPDQQVVFKLPEMIRGGSPVVYDKNKVARFGILDK
YAEDSSNIKWIDAPDCFCFHLWNAWEEPETDEVVVIGSCMTPPDSIFNESDENLKSVLSEIRLNLKT
GESTRRPIISNEDQQVNLEAGMVNRNMLGRKTKFAYLALAEPWPKVSGFAKVDLTTGEVKKHLY
GDNRYGGEPLFLPGEGGEEDEGYILCFVHDEKTWKSELQIVNAVSLEVEATVKLPSRVPYGFHGTF
IGADDLAKQVV*
Promoter YP0384
Modulates the gene: Heat shock transcription factor family.
The GenBank description of the gene: NM_113182 Arabidopsis thaliana heat shock
transcription factor family (At3g22830) mRNA, complete cds
gi|18403537|ref|NM_113182.1|[18403537]
The promoter sequence (SEQ ID NO: 11):
5′ataaaaattcacatttgcaaattttattcagtcggaatatatatttgaaacaagttttgaaatccattg
gacgattaaaattcattgttgagaggataaatatggatttgttcatctgaaccatgtcgttgattagtgat
tgactaccatgaaaaatatgttatgaaaagtataacaacttttgataaatcacatttattaacaataaatc
aagacaaaatatgtcaacaataatagtagtagaagatattaattcaaattcatccgtaacaacaaaaaatc
ataccacaattaagtgtacagaaaaaccttttggatatatttattgtcgcttttcaatgattttcgtgaaa
aggatatatttgtgtaaaataagaaggatcttgacgggtgtaaaaacatgcacaattcttaatttagacca
atcagaagacaacacgaacacttctttattataagctattaaacaaaatcttgcctattttgcttagaata
atatgaagagtgactcatcagggagtggaaaatatctcaggatttgcttttagctctaacatgtcaaacta
tctagatgccaacaacacaaagtgcaaattcttttaatatgaaaacaacaataatatttctaatagaaaat
taaaaagggaaataaaatatttttttaaaatatacaaaagaagaaggaatccatcatcaaagttttataaa
attgtaatataatacaaacttgtttgcttccttgtctctccctctgtctctctcatctctcctatcttctc
catatatacttcatcttcacacccaaaactccacacaaaatatctctccctctatctgcaaattttccaaa
gttgcatcctttcaatttccactcctctctaaTATAattcacattttcccactattgctgattcatttttt
tttgtgaattatttcaaacccacataaaa 3′-TG
The promoter was cloned from the organism: Arabidopsis thaliana, Columbia ecotype

Alternative nucleotides:

| Predicted Position (bp) | Mismatch | Predicted/Experimental |
|---|---|---|
| 18 | SNP | c/- |

The promoter was cloned in the vector: pNewbin4-HAP1-GFP
When cloned into the vector the promoter was operably linked to a marker, which was
the type: GFP-ER
Promoter-marker vector was tested in: Arabidopsis thaliana, WS ecotype
Generation screened: XT1 Mature XT2 Seedling T2 Mature T3 Seedling
The spatial expression of the promoter-marker vector was found observed in and would
be useful in expression in any or all of the following:

| | |
|---|---|
| Primary Root | H epidermis H trichoblast H atrichoblast |

Observed expression pattern of the promoter-marker vector was in:
T1 mature: No expression.
T2 seedling: High expression throughout root epidermal cells.

| | | |
|---|---|---|
| Misc. promoter information: Bidirectionality: Pass | Exons: Pass | Repeats: No |

Optional Promoter Fragments: 5′ UTR region at base pairs 839-999.
The Ceres cDNA ID of the endogenous coding sequence to the promoter: 12730108
cDNA nucleotide sequence (SEQ ID NO: 12):
ACAAAATATCTCTCCCTCTATCTGCAAATTTTCCAAAGTTGCATCCTTTCAATTTCCACTCCTCT
CTAATATAATTCACATTTTCCCACTATTGCTGATTCATTTTTTTTTGTGAATTATTTCAAACCCA
CATAAAAAAATCTTTGTTTAAATTTAAAACCATGGATCCTTCATTTAGGTTCATTAAAGAGGA
GTTTCCTGCTGGATTCAGTGATTCTCCATCACCACCATCTTCTTCTTCATACCTTTATTCATCTT
CCATGGCTGAAGCAGCCATAAATGATCCAACAACATTGAGCTATCCACAACCATTAGAAGGTC
TCCATGAATCAGGGCCACCTCCATTTTTGACAAAGACATATGACTTGGTGGAAGATTCAAGAA
CCAATCATGTCGTGTCTTGGAGCAAATCCAATAACAGCTTCATTGTCTGGGATCCACAGGCCT
TTTCTGTAACTCTCCTTCCCAGATTCTTCAAGCACAATAACTTCTCCAGTTTTGTCCGCCAGCTC
AACACATATGGTTTCAGAAAGGTGAATCCGGATCGGTGGGAGTTTGCAAACGAAGGGTTTCTT
AGAGGGCAAAAGCATCTCCTCAAGAACATAAGGAGAAGAAAAACAAGTAATAATAGTAATCA
AATGCAACAACCTCAAAGTTCTGAACAACAATCTCTAGACAATTTTTGCATAGAAGTGGGTAG
GTACGGTCTAGATGGAGAGATGGACAGCCTAAGGCGAGACAAGCAAGTGTTGATGATGGAGC
TAGTGAGACTAAGACAGCAACAACAAAGCACCAAAATGTATCTCACATTGATTGAAGAGAAG
CTCAAGAAGACCGAGTCAAAACAAAAACAAATGATGAGCTTCCTTGCCCGCGCAATGCAGAA
TCCAGATTTTATTCAGCAGCTAGTAGAGCAGAAGGAAAAGAGGAAAGAGATCGAAGAGGCGA
TCAGCAAGAAGAGACAAAGACCGATCGATCAAGGAAAAAGAAATGTGGAAGATTATGGTGAT
GAAAGTGGTTATGGGAATGATGTTGCAGCCTCATCCTCAGCATTGATTGGTATGAGTCAGGAA
TATACATATGGAAACATGTCTGAATTCGAGATGTCGGAGTTGGACAAACTTGCTATGCACATT
CAAGGACTTGGAGATAATTCCAGTGCTAGGGAAGAAGTCTTGAATGTGGAAAAAGGAAATGA
TGAGGAAGAAGTAGAAGATCAACAACAAGGGTACCATAAGGAGAACAATGAGATTTATGGTG
AAGGTTTTTGGGAAGATTTGTTAAATGAAGGTCAAAATTTTGATTTTGAAGGAGATCAAGAAA
ATGTTGATGTGTTAATTCAGCAACTTGGTTATTTGGGTTCTAGTTCACACACTAATTAAGAAGA
AATTGAAATGATGACTACTTTAAGCATTTGAATCAACTTGTTTCCTATTAGTAATTTGGCTTTG
TTTCAATCAAGTGAGTCGTGGACTAACTTATTGAATTTGGGGGTTAAATCCGTTTCTTATTTTT
GGAAATAAAATTGCTTTTTGTTT
Coding sequence (SEQ ID NO: 13):
MDPSFRFIKEEFPAGFSDSPSPPSSSSYLYSSSMAEAAINDPTTLSYPQPLEGLHESGPPPFLTKTYDL
VEDSRTNHVVSWSKSNNSFIVWDPQAFSVTLLPRFFKHNNFSSFVRQLNTYGFRKVNPDRWEFAN
EGFLRGQKHLLKNIRRRKTSNNSNQMQQPQSSEQQSLDNECIEVGRYGLDGEMDSLRRDKQVLM
MELVRLRQQQQSTKMYLTLIEEKLKKTESKQKQMMSFLARAMQNPDFIQQLVEQKEKRKEIEEAI
SKKRQRPIDQGKRNVEDYGDESGYGNDVAASSSALIGMSQEYTYGNMSEFEMSELDKLAMHIQG
LGDNSSAREEVLNVEKGNDEEEVEDQQQGYHKENNEIYGEGFWEDLLNEGQNEDFEGDQENVDV
LIQQLGYLGSSSHTN*
Promoter YP0382
Modulates the gene: product = “expressed protein”
The GenBank description of the gene: NM_129727 Arabidopsis thaliana expressed protein
(At2g41640) mRNA, complete cds gi|30688728|ref|NM_129727.2|[30688728]
The promoter sequence (SEQ ID NO: 14):
5′ttttttaaaattcgttggaacttggaagggattttaaatattattttgttttccttcatttttataggt
taataattgtcaaagatacaactcgatggaccaaaataaaataataaaattcgtcgaatttggtaaagcaa
aacggtcgaggatagctaatatttatgcgaaacccgttgtcaaagcagatgttcagcgtcacgcacatgcc
gcaaaaagaatatacatcaacctcttttgaacttcacgccgttttttaggcccacaataatgctacgtcgt
cttctgggttcaccctcgttttttttttaaacttctaaccgataaaataaatggtccactatttcttttct
tctctgtgtattgtcgtcagagatggtttaaaagttgaaccgaactataacgattctcttaaaatctgaaa
accaaactgaccgattttcttaactgaaaaaaaaaaaaaaaaaaactgaatttaggccaacttgttgtaat
atcacaaagaaaattctacaatttaattcatttaaaaataaagaaaaatttaggtaacaatttaactaagt
ggtctatctaaatcttgcaaattctttgactttgaccaaacacaacttaagttgacagccgtctcctctct
gttgtttccgtgttattaccgaaatatcagaggaaagtccactaaaccccaaatattaaaaatagaaacat
tactttctttacaaaaggaatctaaattgatccctttcattcgtttcactcgtttcatatagttgtatgta
tatatgcgtatgcatcaaaaagtctcttTATAtcctcagagtcacccaatcttatctctctctccttcgtc
ctcaagaaaagtaattctctgtttgtgtagttttctttaccggtgaattttctcttcgttttgtgcttcaa
acgtcacccaaatcaccaagatcgatcaa 3′-TG
The promoter was cloned from the organism: Arabidopsis thaliana, Columbia ecotype

Alternative nucleotides:

| Predicted Position (bp) | Mismatch | Predicted/Experimental |
|---|---|---|
| 484 | Sequence resolution | a/- |

The promoter was cloned in the vector: pNewbin4-HAP1-GFP
When cloned into the vector the promoter was operably linked to a marker, which was
the type: GFP-ER
Promoter-marker vector was tested in: Arabidopsis thaliana, WS ecotype
Generation screened: XT1 Mature XT2 Seedling T2 Mature T3 Seedling
The spatial expression of the promoter-marker vector was found observed in and would
be useful in expression in any or all of the following:

| | |
|---|---|
| Flower | H nectary M sepal M vascular |
| Primary Root | H epidermis H root cap |

Observed expression pattern:
T1 mature: Expressed in nectary glands of flowers and vasculature of sepals
(see Report 129, Table 1B.).
T2 seedling: High root epidermal expression through to root cap.

| | | |
|---|---|---|
| Misc. promoter information: Bidirectionality: Pass | Exons: Pass | Repeats: No |

Optional Promoter Fragments: 5′ UTR region at base pairs 842-999.
The Ceres cDNA ID of the endogenous coding sequence to the promoter: 12735575
cDNA nucleotide sequence (SEQ ID NO: 15):
AGAGTCACCCAATCTTATCTCTCTCTCCTTCGTCCTCAAGAAAAGTAATTCTCTGTTTGTGTAG
TTTTCTTTACCGGTGAATTTTCTCTTCGTTTTGTGCTTCAAACGTCACCCAAATCACCAAGATC
GATCAAAATCGAAACTTAACGTTTCAGAAGATGGTGCAGTACCAGAGATTAATCATCCACCAT
GGAAGAAAAGAAGATAAGTTTAGAGTTTCTTCAGCAGAGGAAAGTGGTGGAGGTGGTTGTTG
CTACTCCAAGAGAGCTAAACAAAAGTTTCGTTGTCTTCTCTTTCTCTCTATCCTCTCTTGCTGTT
TCGTCTTGTCTCCTTATTACCTCTTCGGCTTCTCTACTCTCTCCCTCCTAGATTCGTTTCGCAGA
GAAATCGAAGGTCTTAGCTCTTATGAGCCAGTTATTACCCCTCTGTGCTCAGAAATCTCCAATG
GAACCATTTGTTGTGACAGAACCGGTTTGAGATCTGATATTTGTGTAATGAAAGGTGATGTTC
GAACAAACTCTGCTTCTTCCTCAATCTTCCTCTTCACCTCCTCCACCAATAACAACACAAAACC
GGAAAAGATCAAACCTTACACTAGAAAATGGGAGACTAGTGTGATGGACACCGTTCAAGAAC
TCAACCTCATCACCAAAGATTCCAACAAATCTTCAGATCGTGTATGCGATGTGTACCATGATG
TTCCTGCTGTGTTCTTCTCCACTGGTGGATACACCGGTAACGTATACCACGAGTTTAACGACGG
GATTATCCCTTTGTTTATAACTTCACAGCATTACAACAAAAAAGTTGTGTTTGTGATCGTCGAG
TATCATGACTGGTGGGAGATGAAGTATGGAGATGTCGTTTCGCAGCTCTCGGATTATCCTCTG
GTTGATTTCAATGGAGATACGAGAACACATTGTTTCAAAGAAGCAACCGTTGGATTACGTATT
CACGACGAGTTAACTGTGAATTCTTCTTTGGTCATTGGGAATCAAACCATTGTTGACTTCAGAA
ACGTTTTGGATAGGGGTTACTCGCATCGTATCCAAAGCTTGACTCAGGAGGAAACAGAGGCGA
ACGTGACCGCACTCGATTTCAAGAAGAAGCCAAAACTGGTGATTCTTTCAAGAAACGGGTCAT
CAAGGGCGATATTAAACGAGAATCTTCTCGTGGAGCTAGCAGAGAAAACAGGGTTCAATGTG
GAGGTTCTAAGACCACAAAAGACAACGGAAATGGCCAAGATTTATCGTTCGTTGAACACGAG
CGATGTAATGATCGGTGTACATGGAGCAGCAATGACTCATTTCCTTTTCTTGAAACCGAAAAC
CGTTTTCATTCAGATCATCCCATTAGGGACGGACTGGGCGGCAGAGACATATTATGGAGAACC
GGCGAAGAAGCTAGGATTGAAGTACGTTGGTTACAAGATTGCGCCGAAAGAGAGCTCTTTGT
ATGAAGAATATGGGAAAGATGACCCTGTAATCCGAGATCCGGATAGTCTAAACGACAAAGGA
TGGGAATATACGAAGAAAATCTATCTACAAGGACAGAACGTGAAGCTTGACTTGAGAAGATT
CAGAGAAACGTTAACTCGTTCGTATGATTTCTCCATTAGAAGGAGATTTAGAGAAGATTACTT
GTTACATAGAGAAGATTAAGAATCGTGTGATATTTTTTTTGTAAAGTTTTGAATGACAATTAA
ATTTATTTATTTTAT
Coding sequence (SEQ ID NO: 16):
MVQYQRLIIHHGRKEDKFRVSSAEESGGGGCCYSKRAKQKFRCLLFLSILSCCFVLSPYYLFGFSTL
SLLDSFRREIEGLSSYEPVITPLCSEISNGTICCDRTGLRSDICVMKGDVRTNSASSSIFLFTSSTNNNT
KPEKIKPYTRKWETSVMDTVQELNLITKDSNKSSDRVCDVYHDVPAVFFSTGGYTGNVYHEFND
GIIPLFITSQHYNKKVVFVIVEYHDWWEMKYGDVVSQLSDYPLVDFNGDTRTHCFKEATVGLRIH
DELTVNSSLVIGNQTIVDFRNVLDRGYSHRIQSLTQEETEANVTALDFKKKPKLVILSRNGSSRAIL
NENLLVELAEKTGFNVEVLRPQKTTEMAKIYRSLNTSDVMIGVHGAAMTHFLFLKPKTVFIQIIPLG
TDWAAETYYGEPAKKLGLKYVGYKIAPKESSLYEEYGKDDPVIRDPDSLNDKGWEYTKKIYLQG
QNVKLDLRRFRETLTRSYDFSIRRRFREDYLLHRED*
Promoter YP0381
Modulates the gene: Unknown expressed protein
The GenBank description of the gene: NM_113878 Arabidopsis thaliana expressed protein
(At3g29575) mRNA, complete cds gi|30689672|ref|NM_113878.3|[30689672]
The promoter sequence (SEQ ID NO: 17):
5′tcattacattgaaaaagaaaattaattgtctttactcatgtttattctatacaaataaaaatatta
accaaccatcgcactaacaaaatagaaatcttattctaatcacttaattgttgacaattaaatcattg
aaaaatacacttaaatgtcaaatattcgttttgcatacttttcaatttaaatacatttaaagttcgac
aagttgcgtttactatcatagaaaactaaatctcctaccaaagcgaaatgaaactactaaagcgacag
gcaggttacataacctaacaaatctccacgtgtcaattaccaagagaaaaaaagagaagataagcgga
acacgtggtagcacaaaaaagataatgtgatttaaattaaaaaacaaaaacaaagacacgtgacgacc
tgacgctgcaacatcccaccttacaacgtaataaccactgaacataagacacgtgtacgatcttgtct
ttgttttctcgatgaaaaccacgtgggtgctcaaagtccttgggtcagagtcttccatgattccacgt
gtcgttaatgcaccaaacaagggtactttcggtattttggcttccgcaaattagacaaaacagctttt
tgtttgattgatttttctcttctctttttccatctaaattctctttgggctcttaatttctttttgag
tgttcgttcgagatttgtcggagattttttcggtaaatgttgaaattttgtgggatttttttttattt
ctttattaaacttttttttattgaattTATAaaaagggaaggtcgtcattaatcgaagaaatggaatc
ttccaaaatttgatattttgctgttttcttgggatttgaattgctctttatcatcaagaatctgttaa
aatttctaatctaaaatctaagttgagaaaaagagagatctctaatttaaccggaattaatattctcc
3′-cATG
The promoter was cloned from the organism: Arabidopsis thaliana, Columbia ecotype

Alternative nucleotides:
Predicted (Columbia)
Experimental (Columbia)

| Predicted Position (bp) | Mismatch | Predicted/Experimental |
|---|---|---|
| 966 | Sequence read error | -/a |

The promoter was cloned in the vector: pNewbin4-HAP1-GFP
When cloned into the vector the promoter was operably linked to a marker, which was
the type: GFP-ER
Promoter-marker vector was tested in: Arabidopsis thaliana, Columbia ecotype
Generation screened: XT1 Mature XT2 Seedling T2 Mature T3 Seedling
The spatial expression of the promoter-marker vector was found observed in and would
be useful in expression in any or all of the following:

| | |
|---|---|
| Flower | L pedicel H nectary L epidermis |
| Hypocotyl | L vascular |
| Primary Root | H vascular |

Observed expression pattern:
T1 mature: High expression in nectary glands of flowers. Low expression in epidermis
of pedicles developing flowers.
T2 seedling: GFP expressed in root and hypocotyl vasculature.

| | | |
|---|---|---|
| Misc. promoter information: Bidirectionality: Pass | Exons: Pass | Repeats: No |

Optional Promoter Fragments: 5′ UTR region at base pairs 671-975.
The Ceres cDNA ID of the endogenous coding sequence to the promoter: 12736859
cDNA nucleotide sequence (SEQ ID NO: 18):
AAATTCTCTTTGGGCTCTTAATTTCTTTTTGAGTGTTCGTTCGAGATTTGTCGGAGATTTTTTCG
GTAAATGTTGAAATTTTGTGGGATTTTTTTTTATTTCTTTATTAAACTTTTTTTTATTGAATTTA
TAAAAAGGGAAGGTCGTCATTAATCGAAGAAATGGAATCTTCCAAAATTTGATATTTTGCTGT
TTTCTTGGGATTTGAATTGCTCTTTATCATCAAGAATCTGTTAAAATTTCTAATCTAAAATCTA
AGTTGAGAAAAAGAGAGATCTCTAATTTAACCGGAATTAATATTCTCCGACCGAAGTTATTAT
GTTGCAGGCTCATGTCGAAGAAACAGAGATTGTCTGAAGAAGATGGAGAGGTAGAGATTGAG
TTAGACTTAGGTCTATCTCTAAATGGAAGATTTGGTGTTGACCCACTTGCGAAAACAAGGCTT
ATGAGGTCTACGTCGGTTCTTGATTTGGTGGTCAACGATAGGTCAGGGCTGAGTAGGACTTGT
TCGTTACCCGTGGAGACGGAGGAAGAGTGGAGGAAGAGGAAGGAGTTGCAGAGTTTGAGGAG
GCTTGAGGCTAAGAGAAAGAGATCAGAGAAGCAGAGGAAACATAAAGCTTGTGGTGGTGAAG
AGAAGGTTGTGGAAGAAGGATCTATTGGTTCTTCTGGTAGTGGTTCCTCTGGTTTGTCTGAAG
TTGATACTCTTCTTCCTCCTGTTCAAGCAACAACGAACAAGTCCGTGGAAACAAGCCCTTCAA
GTGCCCAATCTCAGCCCGAGAATTTGGGCAAAGAAGCGAGCCAAAACATTATAGAGGACATG
CCATTCGTGTCAACAACAGGCGATGGACCGAACGGGAAAAAGATTAATGGGTTTCTGTATCGG
TACCGCAAAGGTGAGGAGGTGAGGATTGTCTGTGTGTGTCATGGAAGCTTCCTCTCACCGGCA
GAATTCGTTAAGCATGCTGGTGGTGGTGACGTTGCACATCCCTTAAAGCACATCGTTGTAAAT
CCATCTCCCTTCTTGTGACCCTTTGGGTCTCTTTTGAGGGGTTTGTTGTATCGGAACCATGTTA
CAAATCCTCATTATCTCCGAGGTGTATAAACATAAATTTATCGAACTCGCAATTTTCAGATTTT
GTACTTAAAAGAATGGTTTCATTCGTTGAGATTAATTTTAGACCTTTTTCTTGTAC
Coding sequence (SEQ ID NO: 19):
MSKKQRLSEEDGEVEIELDLGLSLNGRFGVDPLAKTRLMRSTSVLDLVVNDRSGLSRTCSLPVETE
EEWRKRKELQSLRRLEAKRKRSEKQRKHKACGGEEKVVEEGSIGSSGSGSSGLSEVDTLLPPVQAT
TNKSVETSPSSAQSQPENLGKEASQNIIEDMPFVSTTGDGPNGKKINGFLYRYRKGEEVRTVCVCH
GSFLSPAEFVKHAGGGDVAHPLKHIVVNPSPFL*
Promoter YP0380
Modulates the gene: Responsive to Dehydration 20
The GenBank description of the gene: : NM_128898 Arabidopsis thaliana RD20 protein
(At2g33380) mRNA, complete cds gi|30685670|ref|NM_128898.2|[30685670]
The promoter sequence (SEQ ID NO: 20):
5′tttcaatgtatacaatcatcatgtgataaaaaaaaaaatgtaaccaatcaacacactgagatacggcca
aaaaatggtaatacataaatgtttgtaggttttgtaatttaaatactttagttaagttatgattttattat
ttttgcttatcacttatacgaaatcatcaatctattggtatctcttaatcccgctttttaatttccaccgc
acacgcaaatcagcaaatggttccagccacgtgcatgtgaccacatattgtggtcacagtactcgtccttt
ttttttcttttgtaatcaataaatttcaatcctaaaacttcacacattgagcacgtcggcaacgttagctc
ctaaatcataacgagcaaaaaagttcaaattagggtatatgatcaattgatcatcactacatgtctacata
attaatatgtattcaaccggtcggtttgttgatactcatagttaagtatatatgtgctaattagaattagg
atgaatcagttcttgcaaacaactacggtttcatataatatgggagtgttatgtacaaaatgaaagaggat
ggatcattctgagatgttatgggctcccagtcaatcatgttttgctcgcatatgctatcttttgagtctct
tcctaaactcatagaataagcacgttggttttttccaccgtcctcctcgtgaacaaaagtacaattacatt
ttagcaaattgaaaataaccacgtggatggaccatattatatgtgatcatattgcttgtcgtcttcgtttt
cttttaaatgtttacaccactacttcctgacacgtgtccctattcacatcatccttgttatatcgttttac
tTATAaaggatcacgaacaccaaaacatcaatgtgtacgtcttttgcataagaagaaacagagagcattat
caattattaacaattacacaagacagcga 3′-aATG
The promoter was cloned from the organism: Arabidopsis thaliana, Columbia ecotype

Alternative nucleotides:

| | | |
|---|---|---|
| Predicted Position (bp) | Mismatch | Predicted/Experimental |

| | | |
|---|---|---|
| 5 | PCR error or ecotype variant SNP | g/- correct is -/- |
| 17 | PCR error or ecotype variant SNP | c/- correct is -/- |

The promoter was cloned in the vector: pNewbin4-HAP1-GFP
When cloned into the vector the promoter was operably linked to a marker, which was
the type: GFP-ER
Promoter-marker vector was tested in: Arabidopsis thaliana, WS ecotype
Generation screened: XT1 Mature XT2 Seedling T2 Mature T3 Seedling
The spatial expression of the promoter-marker vector was found observed in and would
be useful in expression in any or all of the following:

| | |
|---|---|
| Flower | H pedicel H receptacle H sepal H petal H filament H anther H carpel |
| | H stigma H epidermis H stomata H silique H style |
| Silique | H stigma H style H carpel H septum H placentae H epidermis |
| Stem | L epidermis L cortex H stomata |
| Leaf | H mesophyll H stomata |
| Hypocotyl | H epidermis H stomata |
| Cotyledon | H mesophyll H epidermis |
| Rosette Leaf | H mesophyll H epidermis |
| Primary Root | H epidermis |

Observed expression pattern:
T1 mature: High expression throughout floral organs. High expression in stem guard
cells and cortex cells surrounding stomal chamber (see Table 1. FIG.P). Not expressed
in shoot apical meristem, early flower primordia, pollen and ovules.
T2 seedling: Expressed in all tissues near seedling apex increasing toward root. High
root epidermis expression.
Optional Promoter Fragments: 5′ UTR region at base pairs 905-1000.

| | | |
|---|---|---|
| Misc. promoter information: Bidirectionality: Pass | Exons: Pass | Repeats: No |

The Ceres cDNA ID of the endogenous coding sequence to the promoter: 12462179
cDNA nucleotide sequence (SEQ ID NO: 21):
AATGTGTACGTCTTTTGCATAAGAAGAAACAGAGAGCATTATCAATTATTAACAATTACACAA
GACAGCGAGATTGTAAAAGAGTAAGAGAGAGAGAATGGCAGGAGAGGCAGAGGCTTTGGCC
ACGACGGCACCGTTAGCTCCGGTCACCAGTCAGCGAAAAGTACGGAACGATTTGGAGGAAAC
ATTACCAAAACCATACATGGCAAGAGCATTAGCAGCTCCAGATACAGAGCATCCGAATGGAA
CAGAAGGTCACGATAGCAAAGGAATGAGTGTTATGCAACAACATGTTGCTTTCTTCGACCAAA
ACGACGATGGAATCGTCTATCCTTGGGAGACTTATAAGGGATTTCGTGACCTTGGTTTCAACC
CAATTTCCTCTATCTTTTGGACCTTACTCATAAACTTAGCGTTCAGCTACGTTACACTTCCGAG
TTGGGTGCCATCACCATTATTGCCGGTTTATATCGACAACATACACAAAGCCAAGCATGGGAG
TGATTCGAGCACCTATGACACCGAAGGAAGGTATGTCCCAGTTAACCTCGAGAACATATTTAG
CAAATACGCGCTAACGGTTAAAGATAAGTTATCATTTAAAGAGGTTTGGAATGTAACCGAGGG
AAATCGAATGGCAATCGATCCTTTTGGATGGCTTTCAAACAAAGTTGAATGGATACTACTCTA
TATTCTTGCTAAGGACGAAGATGGTTTCCTATCTAAAGAAGCTGTGAGAGGTTGCTTTGATGG
AAGTTTATTTGAACAAATTGCCAAAGAGAGGGCCAATTCTCGCAAACAAGACTAAGAATGTGT
GTGTTTGGTTAGCGAATAAAGCTTTTTGAAGAAAAGCATTGTGTAATTTAGCTTCTTTCGTCTT
GTTATTCAGTTTGGGGATTTGTATAATTAATGTGTTTGTAAACTATGTTTCAAAGTTATATAAA
TAAGAGAAGATGTTACAAAAAAAAAAAAAAGACTAATAAGAAGAATTTGGT
Coding sequence (SEQ ID NO: 22):
MAGEAEALATTAPLAPVTSQRKVRNDLEETLPKPYMARALAAPDTEHPNGTEGHDSKGMSVMQ
QHVAFFDQNDDGIVYPWETYKGFRDLGFNPISSIFWTLLINLAFSYVTLPSWVPSPLLPVYIDNIHK
AKHGSDSSTYDTEGRYVPVNLENIFSKYALTVKDKLSFKEVWNVTEGNRMAIDPFGWLSNKVEWI
LLYILAKDEDGFLSKEAVRGCFDGSLFEQIAKERANSRKQD*
Promoter YP00374
Modulates the gene: Putative cytochrome P450
The GenBank description of the gene: NM_112814 Arabidopsis thaliana cytochrome P450,
putative (At3g19270) mRNA, complete cds gi|18402178|ref|NM_112814.1|[18402178]
The promoter sequence (SEQ ID NO: 23):
5′agaagaaactagaaacgttaaacgcatcaaatcaagaaattaaattgaaggtaatttttaacgccgcct
ttcaaatattcttcctaggagaggctacaagacgcgtatttctttcgaattctccaaaccattaccatttt
gatatataataccgacatgccgttgataaagtttgtatgcaaatcgttcattgggtatgagcaaatgccat
ccattggttcttgtaattaaatggtccaaaaatagtttgttcccactactagttactaatttgtatcactc
tgcaaaataatcatgatataaacgtatgtgctatttctaattaaaactcaaaagtaatcaatgtacaatgc
agagatgaccataaaagaacattaaaacactacttccactaaatctatggggtgccttggcaaggcaattg
aataaggagaatgcatcaagatgatatagaaaatgctattcagtttataacattaatgttttggcggaaaa
ttttctatatattagacctttctgtaaaaaaaaaaaaatgatgtagaaaatgctattatgtttcaaaaatt
tcgcactagtataatacggaacattgtagtttacactgctcattaccatgaaaaccaaggcagtatatacc
aacattaataaactaaatcgcgatttctagcacccccattaattaattttactattatacattctctttgc
ttctcgaaataataaacttctctatatcattctacataataaataagaaagaaatcgacaagatctaaatt
tagatctattcagctttttcgcctgagaagccaaaattgtgaatagaagaaagcagtcgtcatcttcccac
gtttggacgaaataaaacataacaataataaaataataaatcaaatatataaatccctaatttgtctttat
tactccacaattttctatgtgtatataTA 3′-

(SEQ ID NO: 24)

tgtatgtttttgttccctattatatcttctagcttctttcttcctcttcttccttaaaaattcatcctcca
aaaca ttctatcatcaacgaaacatttcatattaaattaaataataatcgATG
The promoter was cloned from the organism: Arabidopsis thaliana

Alternative nucleotides:
Query = Predicted
Subject = Experimental

| Predicted Position (bp) | Mismatch | Predicted/Experimental |
|---|---|---|
| 1-1000 | None | Identities = 1000/1000 (100%) |

The promoter was cloned in the vector: pNewbin4-HAP1-GFP
When cloned into the vector the promoter was operably linked to a marker, which was
the type: GFP-ER
Promoter-marker vector was tested in:
Generation screened: XT1 Mature XT2 Seedling T2 Mature T3 Seedling
The spatial expression of the promoter-marker vector was found observed in and would
be useful in expression in any or all of the following:

| | |
|---|---|
| Flower | M vascular |
| Silique | M placenta, M vascular |
| Hypocotyl | H vascular |
| Cotyledon | H vascular, H petiole |
| Primary Root | H vascular |

Observed expression pattern of the promoter-marker vector was in:
T1 mature: GFP expressed in outer integument of developing ovule primordium. Higher
integument expression at chalazal pole observed through maturity.
T2 seedling: Medium to low expression in root vascular bundles weakening toward
hypocotyl.
Weak expression in epidermal cells at root transition zone..

| | | |
|---|---|---|
| Misc. promoter information: Bidirectionality: Pass | Exons: Pass | Repeats: No |

The Ceres cDNA ID of the endogenous coding sequence to the promoter:: 12370888
cDNA nucleotide sequence (SEQ ID NO: 25):
GTATGTTTTTGTTCCCTATTATATCTTCTAGCTTCTTTCTTCCTCTTCTTCCTTAAAAATTCATCC
TCCAAAACATTCTATCATCAACGAAACATTTCATATTAAATTAAATAATAATCGATGGCTGAA
ATTTGGTTCTTGGTTGTACCAATCCTCATCTTATGCTTGCTTTTGGTAAGAGTGATTGTTTCAA
AGAAGAAAAAGAACAGTAGAGGTAAGCTTCCTCCTGGTTCCATGGGATGGCCTTACTTAGGAG
AGACTCTACAACTCTATTCACAAAACCCCAATGTTTTCTTCACCTCCAAGCAAAAGAGATATG
GAGAGATATTCAAAACCCGAATCCTCGGCTATCCATGCGTGATGTTGGCTAGCCCTGAGGCTG
CGAGGTTTGTACTTGTGACTCATGCCCATATGTTCAAACCAACTTATCCGAGAAGCAAAGAGA
AGCTGATAGGACCCTCTGCACTCTTTTTCCACCAAGGAGATTATCATTCCCATATAAGGAAACT
TGTTCAATCCTCTTTCTACCCTGAAACCATCCGTAAACTCATCCCTGATATCGAGCACATTGCC
CTTTCTTCCTTACAATCTTGGGCCAATATGCCGATTGTCTCCACCTACCAGGAGATGAAGAAGT
TCGCCTTTGATGTGGGTATTCTAGCCATATTTGGACATTTGGAGAGTTCTTACAAAGAGATCTT
GAAACATAACTACAATATTGTGGACAAAGGCTACAACTCTTTCCCCATGAGTCTCCCCGGAAC
ATCTTATCACAAAGCTCTCATGGCGAGAAAGCAGCTAAAGACGATAGTAAGCGAGATTATATG
CGAAAGAAGAGAGAAAAGGGCCTTGCAAACGGACTTTCTTGGTCATCTACTCAACTTCAAGAA
CGAAAAAGGTCGTGTGCTAACCCAAGAACAGATTGCAGACAACATCATCGGAGTCCTTTTCGC
CGCACAGGACACGACAGCTAGTTGCTTAACTTGGATTCTTAAGTACTTACATGATGATCAGAA
ACTTCTAGAAGCTGTTAAGGCTGAGCAAAAGGCTATATATGAAGAAAACAGTAGAGAGAAGA
AACCTTTAACATGGAGACAAACGAGGAATATGCCACTGACACATAAGGTTATAGTTGAAAGCT
TGAGGATGGCAAGCATCATATCCTTCACATTCAGAGAAGCAGTGGTTGATGTTGAATATAAGG
GATATTTGATACCTAAGGGATGGAAAGTGATGCCACTGTTTCGGAATATTCATCACAATCCGA
AATATTTTTCAAACCCTGAGGTTTTCGACCCATCTAGATTCGAGGTAAATCCGAAGCCGAATA
CATTCATGCCTTTTGGAAGTGGAGTTCATGCTTGTCCCGGGAACGAACTCGCCAAGTTACAAA
TTCTTATATTTCTCCACCATTTAGTTTCCAATTTCCGATGGGAAGTGAAGGGAGGAGAGAAAG
GAATACAGTACAGTCCATTTCCAATACCTCAAAACGGTCTTCCCGCTACATTTCGTCGACATTC
TCTTTAGTTCCTTAAACCTTTGTAGTAATCTTTGTTGTAGTTAGCCAAATCTAATCCAAATTCG
ATATAAAAAATCCCCTTTCTATTTTTTTTTAAAATCATTGTTGTAGTCTTGAGGGGGTTTAACA
TGTAACAACTATGATGAAGTAAAATGTCGATTCCGGT
Coding sequence (SEQ ID NO: 26):
MAEIWFLVVPILILCLLLVRVIVSKKKKNSRGKLPPGSMGWPYLGETLQLYSQNPNVFFTSKQKRY
GEIFKTRILGYPCVMLASPEAARFVLVTHAHMFKPTYPRSKEKLIGPSALFFHQGDYHSHIRKLVQS
SFYPETIRKLIPDIEHIALSSLQSWANMPIVSTYQEMKKFAFDVGILAIFGHLESSYKEILKHNYNIVD
KGYNSFPMSLPGTSYHKALMARKQLKTIVSEIICERREKRALQTDFLGHLLNEKNEKGRVLTQEQI
ADNIIGVLFAAQDTTASCLTWILKYLHDDQKLLEAVKAEQKAIYEENSREKKPLTWRQTRNMPLT
HKVIVESLRMASIISFTFREAVVDVEYKGYLIPKGWKVMPLFRNIHHNPKYFSNPEVFDPSRFEVNP
KPNTFMPFGSGVHACPGNELAKLQILIFLHHLVSNERWEVKGGEKGIQYSPFPIPQNGLPATFRRHS
L*
Promoter YP0371
Modulates the gene: Unknown protein. Contains putative conserved domains: [ATPase
family associated with various cellular activities (AAA). AAA family proteins often
perform chaperone-like functions that assist in the assembly, operation, or
disassembly of protein complexes]
The GenBank description of the gene: NM_179511 Arabidopsis thaliana AAA-type ATPase
family protein (At1g64110) mRNA, complete cds gi|30696967|ref|NM_179511.1|[30696967].
The promoter sequence (SEQ ID NO: 27):
5′gattctgcgaagacaggagaagccatacctttcaatctaagccgtcaacttgttcccttacgtgggatc
ctattatacaatccaacggttctaaatgagccacgccttccagatctaacacagtcatgctttctacagtc
tgcaccccttttttttttagtgttttatctacattttttcctttgtgtttaattttgtgccaacatctata
acttacccctataaaaatattcaattatcacagaatacccacaatcgaaaacaaaatttaccggaataatt
taattaaagctggactataatgacaattccgaaactatcaaggaataaattaaagaaactaaaaaactaaa
gggcattagagtaaagaagcggcaacatcagaattaaaaaactgccgaaaaaccaacctagtagccgttta
tatgacaacacgtacgcaaagtctcggtaatgactcatcagttttcatgtgcaaacatattacccccatga
aataaaaaagcagagaagcgatcaaaaaaatcttcattaaaagaaccctaaatctctcatatccgccgccg
tctttgcctcattttcaacaccggtgatgacgtgtaaatagatctggttttcacggttctcactactctct
gtgatttttcagactattgaatcgttaggaccaaaacaagtacaaagaaactgcagaagaaaagatttgag
agagatatcttacgaaacaaggtatatatttctcttgttaaatctttgaaaatactttcaaagtttcggtt
ggattctcgaataagttaggttaaatagtcaatatagaattatagataaatcgataccttttgtttgttat
cattcaatttttattgttgttacgattagtaacaacgttttagatcttgatctaTATAttaataatactaa
tactttgtttttttttgttttttttttaa 3′-aATG
The promoter was cloned from the organism: Arabidopsis thaliana, Columbia ecotype

Alternative nucleotides:

| Predicted Position (bp) | Mismatch | Predicted/Experimental |
|---|---|---|
| 155 | PCR error or ecotype variant SNP | t/c |

The promoter was cloned in the vector: pNewbin4-HAP1-GFP
When cloned into the vector the promoter was operably linked to a marker, which was
the type: GFP-ER
Promoter-marker vector was tested in: Arabidopsis thaliana, WS ecotype
Generation screened: XT1 Mature XT2 Seedling T2 Mature T3 Seedling
The spatial expression of the promoter-marker vector was found observed in and would
be useful in expression in any or all of the following:

| | |
|---|---|
| Flower | M pedicel M stomata |
| Primary Root | L epidermis |

Observed expression pattern of the promoter-marker vector was in:
T1 mature: Weak guard cell expression in pedicles.
T2 seedling: Weak root epidermal expression.

| | | |
|---|---|---|
| Misc. promoter information: Bidirectionality: Pass | Exons: Pass | Repeats: No |

An overlap in an exon with the endogenous coding sequence to the promoter occurs at
base pairs 537-754
The Ceres cDNA ID of the endogenous coding sequence to the promoter: 12657397
cDNA nucleotide sequence (SEQ ID NO: 28):
AGCGATCAAAAAAATCTTCATTAAAAGAACCCTAAATCTCTCATATCCGCCGCCGTCTTTGCCT
CATTTTCAACACCGGTGATGACGTGTAAATAGATCTGGTTTTCACGGTTCTCACTACTCTCTGT
GATTTTTCAGACTATTGAATCGTTAGGACCAAAACAAGTACAAAGAAACTGCAGAAGAAAAG
ATTTGAGAGAGATATCTTACGAAACAAGCAAACAGATGTTGTTGTCGGCGCTTGGCGTCGGAG
TTGGAGTAGGTGTGGGTTTAGGCTTGGCTTCTGGTCAAGCCGTCGGAAAATGGGCCGGCGGGA
ACTCGTCGTCAAATAACGCCGTCACGGCGGATAAGATGGAGAAGGAGATACTCCGTCAAGTT
GTTGACGGCAGAGAGAGTAAAATTACTTTCGATGAGTTTCCTTATTATCTCAGTGAACAAACA
CGAGTGCTTCTAACAAGTGCAGCTTATGTCCATTTGAAGCACTTCGATGCTTCAAAATATACG
AGAAACTTGTCTCCAGCTAGCCGAGCCATTCTCTTGTCCGGCCCTGCCGAGCTTTACCAACAA
ATGCTAGCCAAAGCCCTAGCTCATTTCTTCGATGCCAAGTTACTTCTTCTAGACGTCAACGATT
TTGCACTCAAGATACAGAGCAAATACGGCAGTGGAAATACAGAATCATCGTCATTCAAGAGAT
CTCCCTCAGAATCTGCTTTAGAGCAACTATCAGGACTGTTTAGTTCCTTCTCCATCCTTCCTCA
GAGAGAAGAGTCAAAAGCTGGTGGTACCTTGAGGAGGCAAAGCAGTGGTGTGGATATCAAAT
CAAGCTCAATGGAAGGCTCTAGTAATCCTCCAAAGCTTCGTCGAAACTCTTCAGCAGCAGCTA
ATATTAGCAACCTTGCATCTTCCTCAAATCAAGTTTCAGCGCCTTTGAAACGAAGTAGCAGTTG
GTCATTCGATGAAAAGCTTCTCGTCCAATCTTTATATAAGGTCTTGGCCTATGTCTCCAAGGCG
AATCCGATTGTGTTATATCTTCGAGACGTCGAGAACTTTCTGTTCCGCTCACAGAGAACTTACA
ACTTGTTCCAGAAGCTTCTCCAGAAACTCAGTGGACCGGTCCTCATTCTCGGTTCAAGAATTGT
GGACTTGTCAAGCGAAGACGCTCAAGAAATTGATGAGAAGCTCTCTGCTGTTTTCCCTTATAA
TATCGACATAAGACCTCCTGAGGATGAGACTCATCTAGTGAGCTGGAAATCGCAGCTTGAACG
CGACATGAACATGATCCAAACTCAGGACAATAGGAACCATATCATGGAAGTTTTGTCGGAGAA
TGATCTTATATGCGATGACCTTGAATCCATCTCTTTTGAGGACACGAAGGTTTTAAGCAATTAC
ATTGAAGAGATCGTTGTCTCTGCTCTTTCCTATCATCTGATGAACAACAAAGATCCTGAGTACA
GAAACGGAAAACTGGTGATATCTTCTATAAGTTTGTCGCATGGATTCAGTCTCTTCAGAGAAG
GCAAAGCTGGCGGTCGTGAGAAGCTGAAGCAAAAAACTAAGGAGGAATCATCCAAGGAAGTA
AAAGCTGAATCAATCAAGCCGGAGACAAAAACAGAGAGTGTCACCACCGTAAGCAGCAAGGA
AGAACCAGAGAAAGAAGCTAAAGCTGAGAAAGTTACCCCAAAAGCTCCGGAAGTTGCACCGG
ATAACGAGTTTGAGAAACGGATAAGACCGGAAGTAATCCCAGCAGAAGAAATTAACGTCACA
TTCAAAGACATTGGTGCACTTGACGAGATAAAAGAGTCACTACAAGAACTTGTAATGCTTCCT
CTCCGTAGGCCAGACCTCTTCACAGGAGGTCTCTTGAAGCCCTGCAGAGGAATCTTACTCTTC
GGTCCACCGGGTACAGGTAAAACAATGCTAGCTAAAGCCATTGCCAAAGAGGCAGGAGCGAG
TTTCATAAACGTTTCGATGTCAACAATAACTTCGAAATGGTTTGGAGAAGACGAGAAGAATGT
TAGGGCTTTGTTTACTCTAGCTTCGAAGGTGTCACCAACCATAATATTTGTGGATGAAGTTGAT
AGTATGTTGGGACAGAGAACAAGAGTTGGAGAACATGAAGCTATGAGAAAGATCAAGAATGA
GTTTATGAGTCATTGGGATGGGTTAATGACTAAACCTGGTGAACGTATCTTAGTCCTTGCTGCT
ACTAATCGGCCTTTCGATCTTGATGAAGCCATTATCAGACGATTCGAACGAAGGATCATGGTG
GGACTACCGGCTGTAGAGAACAGAGAAAAGATTCTAAGAACATTGTTGGCGAAGGAGAAAGT
AGATGAAAACTTGGATTACAAGGAACTAGCAATGATGACAGAAGGATACACAGGAAGTGATC
TTAAGAATCTGTGCACAACCGCTGCGTATAGGCCGGTGAGAGAACTTATACAGCAAGAGAGG
ATCAAAGACACAGAGAAGAAGAAGCAGAGAGAGCCTACAAAAGCAGGTGAAGAAGATGAAG
GAAAAGAAGAGAGAGTTATAACACTTCGTCCGTTGAACAGACAAGACTTTAAAGAAGCCAAG
AATCAGGTGGCGGCGAGTTTTGCGGCTGAGGGAGCGGGAATGGGAGAGTTGAAGCAGTGGAA
TGAATTGTATGGAGAAGGAGGATCGAGGAAGAAAGAACAACTCACTTACTTCTTGTAATGATG
ATGATGAATCATGATGCTGGTAATGGATTATGAAATTTGGTAATGTAATAGTATGGTGAATTT
TTGTTTCCATGGTTAATAAGAGAATAAGAATATGATGATATTGCTAAAAGTTTGACCCGT
Coding sequence (SEQ ID NO: 29):
MLLSALGVGVGVGVGLGLASGQAVGKWAGGNSSSNNAVTADKMEKEILRQVVDGRESKITFDEF
PYYLSEQTRVLLTSAAYVHLKHFDASKYTRNLSPASRAILLSGPAELYQQMLAKALAHFFDAKLLL
LDVNDFALKIQSKYGSGNTESSSFKRSPSESALEQLSGLFSSFSILPQREESKAGGTLRRQSSGVDIKS
SSMEGSSNPPKLRRNSSAAANISNLASSSNQVSAPLKRSSSWSFDEKLLVQSLYKVLAYVSKANPIV
LYLRDVENFLFRSQRTYNLFQKLLQKLSGPVLILGSRIVDLSSEDAQEIDEKLSAVFPYNIDIRPPEDE
THLVSWKSQLERDMNMIQTQDNRNHIMEVLSENDLICDDLESISFEDTKVLSNYIEEIVVSALSYHL
MNNKDPEYRNGKLVISSISLSHGFSLFREGKAGGREKLKQKTKEESSKEVKAESIKPETKTESVTTV
SSKEEPEKEAKAEKVTPKAPEVAPDNEFEKRIRPEVIPAEEINVTFKDIGALDEIKESLQELVMLPLR
RPDLFTGGLLKPCRGILLFGPPGTGKTMLAKAIAKEAGASFINVSMSTITSKWFGEDEKNVRALFTL
ASKVSPTIIFVDEVDSMLGQRTRVGEHEAMRKIKNEFMSHWDGLMTKPGERILVLAATNRPFDLD
EAIIRRFERRIMVGLPAVENREKILRTLLAKEKVDENLDYKELAMMTEGYTGSDLKNLCTTAAYRP
VRELIQQERIKDTEKKKQREPTKAGEEDEGKEERVITLRPLNRQDFKEAKNQVAASFAAEGAGMG
ELKQWNELYGEGGSRKKEQLTYFL*
Promoter YP0356
Modulates the gene: Dehydration-induced protein RD22
The GenBank description of the geneN NM_122472 Arabidopsis thaliana dehydration-
induced protein RD22 (At5g25610) mRNA, complete cds
gi|30689960|ref|NM_122472.2|[30689960]
The promoter sequence (SEQ ID NO: 30):
5′tacttgcaaccactttgtaggaccattaactgcaaaataagaattctctaagcttcacaaggggttcgt
ttggtgctataaaaacattgttttaagaactggtttactggttctataaatctataaatccaaatatgaag
tatggcaataataataacatgttagcacaaaaaatactcattaaattcctacccaaaaaaaatctttatat
gaaactaaaacttatatacacaataatagtgatacaaagtaggtcttgatattcaactattcgggattttc
tggtttcgagtaattcgtataaaaggtttaagatctattatgttcactgaaatcttaactttgttttgttt
ccagttttaactagtagaaattgaaagttttaaaaattgttacttacaataaaatttgaatcaatatcctt
aatcaaaggatcttaagactagcacaattaaaacatataacgtagaatatctgaaataactcgaaaatatc
tgaactaagttagtagttttaaaatataatcccggtttggaccgggcagtatgtacttcaatacttgtggg
ttttgacgattttggatcggattgggcgggccagccagattgatctattacaaatttcacctgtcaacgct
aactccgaacttaatcaaagattttgagctaaggaaaactaatcagtgatcacccaaagaaaacattcgtg
aataattgtttgctttccatggcagcaaaacaaataggacccaaataggaatgtcaaaaaaaagaaagaca
cgaaacgaagtagtataacgtaacacacaaaaataaactagagatattaaaaacacatgtccacacatgga
tacaagagcatttaaggagcagaaggcacgtagtggttagaaggtatgtgatataattaatcggcccaaat
agattggtaagtagtagccgtcTATAtca 3′-

(SEQ ID NO: 31)

cagctcctttctactaaaacccttttactataaattctacgtacacgtaccacttcttctcctcaaattca
tcaaacccatttctattccaactcccaaaaATG
The promoter was cloned from the organism: Arabidopsis thaliana, WS ecotype

Alternative nucleotides:
Predicted (Columbia)
Experimental (Wassilewskija)

| Predicted Position (bp) | Mismatch | Columbia/Wassilewskija |
|---|---|---|
| 405 | SNP | g/t |

The promoter was cloned in the vector: pNewbin4-HAP1-GFP
When cloned into the vector the promoter was operably linked to a marker, which was
the type: GFP-ER
Promoter-marker vector was tested in: Arabidopsis thaliana, WS ecotype
Generation screened: XT1 Mature XT2 Seedling T2 Mature T3 Seedling
The spatial expression of the promoter-marker vector was found observed in and would
be useful in expression in any or all of the following:

| | |
|---|---|
| Flower | H pedicel H petal H epidermis |
| Silique | H stigma L style L carpel L septum L epidermis |
| Ovule | H outer integument |
| Stem | H epidermis H stomata |
| Hypocotyl | H epidermis |
| Cotyledon | H epidermis |
| Rosette Leaf | H epidermis H trichome |

Observed expression pattern of the promoter-marker vector was in:
T1 mature: GFP expression specific to epidermal call types. High GFP expression in
epidermis of stem decreasing toward pedicles and inflorescence apex. In the flower,
high expression observed in epidermal cells of petals and stigma, and lower expres-
sion in carpels. High expression in outer integuments of matureing ovules. High
expression throughout epidermal cells of mature lower stem.
T2 seedling: GFP expression specific to epidermal cell types. High expression in
epidermis of hypocotyl, cotyledon, and trichomes of rosette leaves. Not detected in
root.

| | | |
|---|---|---|
| Misc. promoter information: Bidirectionality: Pass | Exons: Pass | Repeats: None: |

The Ceres cDNA ID of the endogenous coding sequence to the promoter: 12394809
cDNA nucleotide sequence (SEQ ID NO: 32):
agCTCCTTTCTACTAAAACCCTTTTACTATAAATTCTACGTACACGTACCACTTCTTCTCCTCAA
ATTCATCAAACCCATTTCTATTCCAACTCCCAAAAATGGCGATTCGTCTTCCTCTGATCTGTCT
TCTTGGTTCATTCATGGTAGTGGCGATTGCGGCTGATTTAACACCGGAGCGTTATTGGAGCAC
TGCTTTACCAAACACTCCCATTCCCAACTCTCTCCATAATCTTTTGACTTTCGATTTTACCGACG
AGAAAAGTACCAACGTCCAAGTAGGTAAAGGCGGAGTAAACGTTAACACCCATAAAGGTAAA
ACCGGTAGCGGAACCGCCGTGAACGTTGGAAAGGGAGGTGTACGCGTGGACACAGGCAAGGG
CAAGCCCGGAGGAGGGACACACGTGAGCGTTGGCAGCGGAAAAGGTCACGGAGGTGGCGTCG
CAGTCCACACGGGTAAACCCGGTAAAAGAACCGACGTAGGAGTCGGTAAAGGCGGTGTGACG
GTGCACACGCGCCACAAGGGAAGACCGATTTACGTTGGTGTGAAACCAGGAGCAAACCCTTTC
GTGTATAACTATGCAGCGAAGGAGACTCAGCTCCACGACGATCCTAACGCGGCTCTCTTCTTC
TTGGAGAAGGACTTGGTTCGCGGGAAAGAAATGAATGTCCGGTTTAACGCTGAGGATGGTTA
CGGAGGCAAAACTGCGTTCTTGCCACGTGGAGAGGCTGAAACGGTGCCTTTTGGATCGGAGA
AGTTTTCGGAGACGTTGAAACGTTTCTCGGTGGAAGCTGGTTCGGAAGAAGCGGAGATGATG
AAGAAGACCATTGAGGAGTGTGAAGCCAGAAAAGTTAGTGGAGAGGAGAAGTATTGTGCGAC
GTCTTTGGAGTCGATGGTCGACTTTAGTGTTTCGAAACTTGGTAAATATCACGTCAGGGCTGTT
TCCACTGAGGTGGCTAAGAAGAACGCACCGATGCAGAAGTACAAAATCGCGGCGGCTGGGGT
AAAGAAGTTGTCTGACGATAAATCTGTGGTGTGTCACAAACAGAAGTACCCATTCGCGGTGTT
CTACTGCCACAAGGCGATGATGACGACCGTCTACGCGGTTCCGCTCGAGGGAGAGAACGGGA
TGCGAGCTAAAGCAGTTGCGGTATGCCACAAGAACACCTCAGCTTGGAACCCAAACCACTTGG
CCTTCAAAGTCTTAAAGGTGAAGCCAGGGACCGTTCCGGTCTGCCACTTCCTCCCGGAGACTC
ATGTTGTGTGGTTCAGCTACTAGATAGATCTGTTTTCTATCTTATTGTGGGTTATGTATAATTA
CGTTTCAGATAATCTATCTTTTGGGATGTTTTGGTTATGAATATACATACATATACATATAGTA
ATGCGTGGTTTCCATATAAGAGTGAAGGCATCTATATGTTTTTTTTTTTATTAACCTACGTAGC
TGTCTTTTGTGGTCTGTATCTTGTGGTTTTGCAAAAACCTATAATAAAATTAGAGCTGAAATGT
TACCATTTC
Coding sequence (SEQ ID NO: 33):
<MAIRLPLICLLGSFMVVAIA>
ADLTPERYWSTALPNTPIPNSLHNLLTFDFTDEKSTNVQVGKGGVNVNTHKGKTGSGTAVNVGK
GGVRVDTGKGKPGGGTHVSVGSGKGHGGGVAVHTGKPGKRTDVGVGKGGVTVHTRHKGRPIY
VGVKPGANPFVYNYAAKETQLHDDPNAALFFLEKDLVRGKEMNVRFNAEDGYGGKTAFLPRGE
AETVPFGSEKFSETLKRFSVEAGSEEAEMMKKTIEECEARKVSGEEKYCATSLESMVDFSVSKLGK
YHVRAVSTEVAKKNAPMQKYKIAAAGVKKLSDDKSVVCHKQKYPFAVFYCHKAMMTTVYAVP
LEGENGMRAKAVAVCHKNTSAWNPNHLAFKVLKVKPGTVPVCHFLPETHVVWFSY*
Promoter YP0337
Modulates the gene: Unknown protein.
The GenBank description of the gene: NM_101546 Arabidopsis thaliana expressed protein
(At1g16850) mRNA, complete cds gi|18394408|ref|NM_101546.1|[18394408]
The promoter sequence (SEQ ID NO: 34):

(SEQ ID NO: 35)

5′acttattagtttaggtttccatcacctatttaattcgtaattcttatacatgcatataatagagataca
tatatacaaatttatgatcatttttgcacaacatgtgatctcattcattagtatgcattatgcgaaaacct
cgacgcgcaaaagacacgtaatagctaataatgttactcatttataatgattgaagcaagacgaaaacaac
aacatatatatcaaattgtaaactagatatttcttaaaagtgaaaaaaaacaaagaaatataaaggacaat
tttgagtcagtctcttaatattaaaacatatatacataaataagcacaaacgtggttacctgtcttcatgc
aatgtggactttagtttatctaatcaaaatcaaaataaaaggtgtaatagttctcgtcatttttcaaattt
taaaaatcagaaccaagtgatttttgtttgagtattgatccattgtttaaacaatttaacacagtatatac
gtctcttgagatgttgacatgatgataaaatacgagatcgtctcttggttttcgaattttgaactttaata
gtttttttttttagggaaactttaatagttgtttatcataagattagtcacctaatggttacgttgcagta
ccgaaccaattttttacccttttttctaaatgtggtcgtggcataatttccaaaagagatccaaaacccgg
tttgctcaactgataagccggtcggttctggtttgaaaaacaagaaataatctgaaagtgtgaaacagcaa
cgtgtctcggtgtttcatgagccacctgccacctcattcacgtcggtcattttgtcgtttcacggttcacg
ctctagacacgtgctctgtccccaccatgactttcgctgccgactcgcttcgctttgcaaactcaaacatg
tgtgTATAtgtaagtttcatcctaataag 3′-caaagaaaacatcaaaATG
The promoter was cloned from the organism: Arabidopsis thaliana, WS ecotype

Alternative nucleotides:
Predicted (Columbia)
Experimenral (Wassilewskija)

| Sequence (bp) | Mismatch | Columbia/Wassilewskija |
|---|---|---|
| 597 | SNP | t/c |
| 996 | SNP | t/a |

The promoter was cloned in the vector: pNewbin4-HAP1-GFP
When cloned into the vector the promoter was operably linked to a marker, which was
the type: GFP-ER
Promoter-marker vector was tested in: Arabidopsis thaliana, WS ecotype
Generation screened: XT1 Mature XT2 Seedling T2 Mature T3 Seedling
The spatial expression of the promoter-marker vector was found observed in and would
be useful in expression in any or all of the following:

| | |
|---|---|
| Primary Root | L epidermis L trichoblast L atrichoblast L root hair |

Observed expression pattern of the promoter-marker vector was in:
T1 mature: No expression.
T2 seedling: Low expression in root epidermal cells at transition zone decreasing to
expression in single cells at mid root

| | | |
|---|---|---|
| Misc. promoter information: Bidirectionality: Pass | Exons: Pass | Repeats: No |

The Ceres cDNA ID of the endogenous coding sequence to the promoter: 12326510
cDNA nucleotide sequence (SEQ ID NO: 36):
ACCACATTAATTTAAAACAAAGAAAACATCAAAATGGCTGAAAAAGTAAAGTCTGGTCAAGTT
TTTAACCTATTATGCATATTCTCGATCTTTTTCTTCCTCTTTGTGTTATCAGTGAATGTTTCGGC
TGATGTCGATTCTGAGAGAGCGGTGCCATCTGAAGATAAAACGACGACTGTTTGGCTAACTAA
AATCAAACGGTCCGGTAAAAATTATTGGGCTAAAGTTAGAGAGACTTTGGATCGTGGACAGTC
CCACTTCTTTCCTCCGAACACATATTTTACCGGAAAGAATGATGCGCCGATGGGAGCCGGTGA
AAATATGAAAGAGGCGGCGACGAGGAGCTTTGAGCATAGCAAAGCGACGGTGGAGGAAGCTG
CTAGATCAGCGGCAGAAGTGGTGAGTGATACGGCGGAAGCTGTGAAAGAAAAGGTGAAGAGG
AGCGTTTCCGGTGGAGTGACGCAGCCGTCGGAGGGATCTGAGGAGCTATAAATACGCAGTTGT
TCTAAGCTTATGGGTTTTAATTATTTAAATAATTAGTGTGTGTTTGAGATCAAAATGACACAGT
TTTGGGGGAGTATATCTCCACATCATATGTTGTTTGCATCACATGGTTTCTCTGTATACAACGA
CCAGATCCACATCACTCATTCTCGTCCTTCTTTTTGTCATGAATACAGAATAATATTTTAGATT
CTAC
Coding sequence (SEQ ID NO: 37):
MAEKVKSGQVFNLLCIFSIFFFLFVLSVNVSADVDSERAVPSEDKTTTVWLTKIKRSGKNYWAKVR
ETLDRGQSHFFPPNTYFTGKNDAPMGAGENMKEAATRSFEHSKATVEEAARSAAEVVSDTAEAV
KEKVKRSVSGGVTQPSEGSEEL*
Promoter YP0289
Modulates the gene: phi-1-related protein
The GenBank description of the gene: NM_125822 Arabidopsis thaliana phi-1-related
protein (At5g64260) mRNA, complete cds gi|30697983|ref|NM_125822.2|[30697983]
The promoter sequence (SEQ ID NO: 38):

(SEQ ID NO: 39)

5′caaacaattactgctcaatgtatttgcgtatagagcatgtccaataccatgcctcatgatgtgagattg
cgaggcggagtcagagaacgagttaaagtgacgacgttttttttgttttttttgggcatagtgtaaagtga
tattaaaatttcatggttggcaggtgactgaaaataaaaatgtgtataggatgtgtttatatgctgacgga
aaaatagttactcaactaatacagatctttataaagagtatataagtctatggttaatcatgaatggcaat
atataagagtagatgagatttatgtttatattgaaacaagggaaagatatgtgtaattgaaacaatggcaa
aatataagtcaaatcaaactggtttctgataatatatgtgttgaatcaatgtatatcttggtattcaaaac
caaaacaactacaccaatttctttaaaaaaccagttgatctaataactacattttaatactagtagctatt
agctgaatttcataatcaatttcttgcattaaaatttaaagtgggttttgcatttaaacttactcggtttg
tattaatagactttcaaagattaaaagaaaactactgcattcagagaataaagctatcttactaaacacta
cttttaaagttcttttttcacttattaatcttcttttacaaatggatctgtctctcctgcatggcaaaata
tcttacactaattttattttctttgtttgataacaaatttatcggctaagcatcacttaaatttaatacac
gttatgaagacttaaaccacgtcacacTATAagaaccttacaggctgtcaaacacccttccctacccactc
acatctctccacgtggcaatctttgatattgacaccttagccactacagctgtcacactcctctctcggtt
tcaaaacaacatctctggtataaata 3′-
aatcaaaacctctcctatatctcttcaatctgatataactacccttctcaATG
The promoter was cloned from the organism: Arabidopsis thaliana, WS ecotype

Alternative nucleotides:
Predicted (Columbia)
Experimental (Wassilewskija)

| | | |
|---|---|---|
| Predicted Position (bp) | Mismatch | Columbia/Wassilewskija |

| | | |
|---|---|---|
| 138 | SNP | t/- |
| 529 | SNP | a/t |
| 561 | SNP | a/g |
| 666 | Read Error | c/c |
| 702 | SNP | t/a |
| 820 | SNP | t/a |

The promoter was cloned in the vector: pNewbin4-HAP1-GFP
When cloned into the vector the promoter was operably linked to a marker, which was
the type: GFP-ER
Promoter-marker vector was tested in: Arabidopsis thaliana, WS ecotype
Generation screened: XT1 Mature XT2 Seedling T2 Mature T3 Seedling
The spatial expression of the promoter-marker vector was found observed in and would
be useful in expression in any or all of the following:

| | |
|---|---|
| Flower | L anther |
| Ovule | Post-fertilization: L endothelium |
| Cotyledon | H epidermis H petiole |
| Rosette Leaf | H trichome |
| Primary Root | H epidermis H root hairs |

Observed expression pattern of the promoter-marker vector was in:
Expression very weak and may not have been detected by standard screen. Only tissue
with visible GFP expression is analyzed by confocal microscopy. This may account for
the expressing/screened ratio.
T1 mature: Low GFP expression in endothelium cells of mature ovules and tapetum cell
layer of anthers.
Not expressed in pollen. T2 seedling: High GFP expression specific to epidermal
tissues of cotyledons, root and trichomes of rosette leaves.

| | | |
|---|---|---|
| Misc. promoter information: Bidirectionality: | Exons: | Repeats: |

The Ceres cDNA ID of the endogenous coding sequence to the promoter: 12326995
cDNA nucleotide sequence (SEQ ID NO: 40):
aaatcaaaacctctcctatatctcttcaatctgatataactacccttctcaatggcttctaattaccgttt
tgccatcttcctcactctctttttcgccaccgctggtttctccgccgccgcgttggtcgaggagcagccgc
ttgttatgaaataccacaacggagttctgttgaaaggtaacatcacagtcaatctcgtatggtacgggaaa
ttcacaccgatccaacggtccgtaatcgtcgatttcatccactcgctaaactccaaagacgttgcatcttc
cgccgcagttccttccgttgcttcgtggtggaagacgacggagaaatacaaaggtggctcttcaacactcg
tcgtcgggaaacagcttctactcgagaactatcctctcggaaaatctctcaaaaatccttacctccgtgct
ttatccaccaaacttaacggcggtctccgttccataaccgtcgttctaacggcgaaagatgttaccgtcga
aagattctgtatgagccggtgcgggactcacggatcctccggttcgaatccccgtcgcgcagctaacggcg
cggcttacgtatgggtcgggaactccgagacgcagtgccctggatattgcgcgtggccgtttcaccagccg
atttacggaccacaaacgccgccgttagtagcgcctaacggtgacgttggagttgacggaatgattataaa
ccttgccacacttctagctaacaccgtgacgaatccgtttaataacggatattaccaaggcccaccaactg
caccgcttgaagctgtgtctgcttgtcctggtatattcgggtcaggttcttatccgggttacgcgggtcgg
gtacttgttgacaaaacaaccgggtctagttacaacgctcgtggactcgccggtaggaaatatctattgcc
ggcgatgtgggatccgcagagttcgacgtgcaagactctggtttgatccaagggatgtgagtaagacacgt
ggcatagtagtgagagcgatgacgagatctagacggcatgtgtagtcaaaatcaagttgcacgcgagcgtg
tgtataaaaaaatctttcgggtttgggtctcgggtttggattgtggatagggctctctctttgctttttgt
cgttttgtaatgacgtgtaaaaactgtactcggaaatgtgaagaatgcatataaaataataaaaaatcatt
ttgttctact
Coding sequence (SEQ ID NO: 41):
MASNYRFAIFLTLFFATAGFSAAALVEEQPLVMKYHNGVLLKGNITVNLVWYGKFTPIQRSVIVDF
IHSLNSKDVASSAAVPSVASWWKTTEKYKGGSSTLVVGKQLLLENYPLGKSLKNPYLRALSTKLN
GGLRSITVVLTAKDVTVERFCMSRCGTHGSSGSNPRRAANGAAYVWVGNSETQCPGYCAWPFHQ
PIYGPQTPPLVAPNGDVGVDGMIINLATLLANTVTNPFNNGYYQGPPTAPLEAVSACPGIFGSGSYP
GYAGRVLVDKTTGSSYNARGLAGRKYLLPAMWDPQSSTCKTLV*
Promoter YP0286
Modulates the gene: Hypothetical protein
The GenBank description of the gene: NM_102758 Arabidopsis thaliana hypothetical
protein (At1g30190) mRNA, complete cds gi|18397396|ref|NM_102758.1|[18397396]
The promoter sequence (SEQ ID NO: 42):
5′atcatcgaaaggtatgtgatgcatattcccattgaaccagatttccatatattttatttgtaaagtgat
aatgaatcacaagatgattcaatattaaaaatgggtaactcactttgacgtgtagtacgtggaagaatagt
tagctatcacgcatatatatatctatgattaagtgtgtatgacataagaaactaaaatatttacctaaagt
ccagttactcatactgattttatgcatatatgtattatttatttatttttaataaagaagcgattggtgtt
ttcatagaaatcatgatagattgataggtatttcagttccacaaatctagatctgtgtgctatacatgcat
gtattaattttttccccttaaatcatttcagttgataatattgctctttgttccaactttagaaaaggtat
gaaccaacctgacgattaacaagtaaacattaattaatctttatatatatgagataaaaccgaggatatat
atgattgtgttgctgtctattgatgatgtgtcgatattatgcttgttgtaccaatgctcgagccgagcgtg
atcgatgccttgacaaactatatatgtttcccgaattaattaagttttgtatcttaattagaataacattt
ttatacaatgtaatttctcaagcagacaagatatgtatcctatattaattactatatatgaattgccgggc
acctaccaggatgtttcaaatacgagagcccattagtttccacgtaaatcacaatgacgcgacaaaatcta
gaatcgtgtcaaaactctatcaatacaataatatatatttcaagggcaatttcgacttctcctcaactcaa
tgattcaacgccatgaatctctaTATAaaggctacaacaccacaaaggatcatcagtcatcacaaccacat
taactcttcaccactatctctcaatctct 3′-ATG
The promoter was cloned from the organism: Arabidopsis thaliana, WS ecotype

Alternative nucleotides:
Predicred (Columbia)
Experimenral (Wassilewskija)

| | | |
|---|---|---|
| Predicted Position (bp) | Mismatch | Columbia/Wassilewskija |

| | | |
|---|---|---|
| 194 | SNP | t/a |
| 257 | SNP | t/c |
| 491-494 | SSLP | tata/---- |
| 527 | No g in Ws | -/- |

The promoter was cloned in the vector: pNewbin4-HAP1-GFP
When cloned into the vector the promoter was operably linked to a marker, which was
the type: GFP-ER
Promoter-marker vector was tested in: Arabidopsis thaliana, WS ecotype
Generation screened: XT1 Mature XT2 Seedling T2 Mature T3 Seedling
The spatial expression of the promoter-marker vector was found observed in and would
be useful in expression in any or all of the following:

| | |
|---|---|
| Flower | L pedicel L epidermis |
| Stem | L epidermis |
| Hypocotyl | H epidermis |
| Cotyledon | H mesophyll H vascular H epidermis H petiole |
| Rosette Leaf | H epidermis H petiole |
| Primary Root | H epidermis |
| Lateral root | H lateral root cap |

Observed expression pattern of the promoter-marker vector was in:
T1 mature: GFP expressed in vasculature of silique and pedicles of flowers.
T2 seedling: High GFP expression throughout vasculature of root, hypocotyl, and
petioles.

| | | |
|---|---|---|
| Misc. promoter information: Bidirectionality: Pass | Exons: Pass | Repeats: No |

The Ceres cDNA ID of the endogenous coding sequence to the promoter: 12669548
cDNA nucleotide sequence (SEQ ID NO: 43):
ATGACAGAAATGCCCTCGTACATGATCGAGAACCCAAAGTTCGAGCCAAAGAAACGACGTTAT
TACTCTTCTTCGATGCTTACCATCTTCTTACCGATCTTCACATACATTATGATCTTTCACGTTTT
CGAAGTATCACTATCTTCGGTCTTTAAAGACACAAAGGTCTTGTTCTTCATCTCCAATACTCTC
ATCCTCATAATAGCCGCCGATTATGGTTCCTTCTCTGATAAAGAGAGTCAAGACTTTTACGGTG
AATACACTGTCGCAGCGGCAACGATGCGAAACCGAGCTGATAACTACTCTCCGATTCCCGTCT
TGACATACCGAGAAAACACTAAAGATGGAGAAATCAAGAACCCTAAAGATGTCGAATTCAGG
AACCCTGAAGAAGAAGACGAACCGATGGTGAAAGATATCATTTGCGTTTCTCCTCCCGAGAAA
ATAGTACGAGTGGTGAGTGAGAAGAAACAGAGAGATGATGTAGCTATGGAAGAATACAAACC
AGTTACAGAACAAACTCTTGCTAGCGAAGAAGCTTGCAACACAAGAAACCATGTGAACCCTAA
TAAACCGTACGGGCGAAGTAAATCAGATAAGCCACGGAGAAAGAGGCTCAGCGTAGATACAG
AGACGACCAAACGTAAAAGTTATGGTCGAAAGAAATCAGATTGCTCGAGATGGATGGTTATTC
CGGAGAAGTGGGAATATGTTAAAGAAGAATCTGAAGAGTTTTCAAAGTTGTCCAACGAGGAG
TTGAACAAACGAGTCGAAGAATTCATCCAACGGTTCAATAGACAGATCAGATCACAATCACCG
CGAGTTTCGTCTACTTGA
Coding sequence (SEQ ID NO: 44):
MTEMPSYMIENPKFEPKKRRYYSSSMLTIFLPIFTYIMIFHVFEVSLSSVFKDTKVLFFI
SNTLILIIAADYGSFSDKESQDFYGEYTVAAATMRNRADNYSPIPVLTYRENTKDGEIKN
PKDVEFRNPEEEDEPMVKDIICVSPPEKIVRVVSEKKQRDDVAMEEYKPVTEQTLASEEA
CNTRNHVNPNKPYGRSKSDKPRRKRLSVDTETTKRKSYGRKKSDCSRWMVIPEKWEYVKE
ESEEFSKLSNEELNKRVEEFIQRFNRQIRSQSPRVSST*
Promoter YP0275
Modulates the gene: Glycosyl hydrolase family.
The GenBank description of the gene: NM_115876 Arabidopsis thaliana glycosyl
hydrolase family 1 (At3g60130) mRNA, complete cds
gi|30695130|ref|NM_115876.2|[30695130]
The promoter sequence (SEQ ID NO: 45):
5′gcgtatgctttactttttaaaatgggcctatgctataattgaatgacaaggattaaacaactaataaaa
gtgtagatgggttaagatgacttatttttttacttaccaatttataaatgggcttcgatgtactgaaatat
atcgcgcctattaacgaggccattcaacgaatgttttaagggccctatttcgacattttaaagaacaccta
ggtcatcattccagaaatggatattataggatttagataatttcccacgtttggtttatttatctattttt
tgacgttgaccaacataatcgtgcccaaccgtttcacgcaacgaatttatatacgaaatatatatattttt
caaattaagataccacaatcaaaacagctgttgattaacaaagagattttttttttttggttttgagttac
aataacgttagaggataaggtttcttgcaacgattaggaaatcgtataaaataaaatatgttataattaag
tgttttattttataatgagtattaatataaataaaacctgcaaaaggatagggatattgaataataaagag
aaacgaaagagcaattttacttctttataattgaaattatgtgaatgttatgtttacaatgaatgattcat
cgttctatatattgaagtaaagaatgagtttattgtgcttgcataatgacgttaacttcacatatacactt
attacataacatttatcacatgtgcgtctttttttttttttactttgtaaaatttcctcactttaaagact
tttataacaattactagtaaaataaagttgcttggggctacaccctttctccctccaacaactctatttat
agataacattatatcaaaatcaaaacatagtccctttcttctataaaggttttttcacaaccaaatttcca
tTATAaatcaaaaaataaaaacttaatta 3′-aATG
The promoter was cloned from the organism: Arabidopsis thaliana, WS ecotype

Alternative nucleotides:
Predicred (Columbia)
Experimental (Wassilewskija)

| | | |
|---|---|---|
| Sequence (bp) | Mismatch | Columbia/Wassilewskija |

| | | |
|---|---|---|
| 95 | SNP | g/t |
| 798 | SNP | a/t |

The promoter was cloned in the vector: pNewbin4-HAP1-GFP
When cloned into the vector the promoter was operably linked to a marker, which was
the type: GFP-ER
Promoter-marker vector was tested in: Arabidopsis thaliana, WS ecotype
Generation screened: XT1 Mature XT2 Seedling T2 Mature T3 Seedling
The spatial expression of the promoter-marker vector was found observed in and would
be useful in expression in any or all of the following:

| | |
|---|---|
| Primary Root | H epidermis H trichoblast H atrichoblast L root cap H root hairs |

Observed expression pattern of the promoter-marker vector was in:
T1 mature: No expression.
T2 seedling: High expression in root epidermal at transition zone decreasing toward
root tip.

| | | |
|---|---|---|
| Misc. promoter information: Bidirectionality: Pass | Exons: Pass | Repeats: No |

The Ceres cDNA ID of the endogenous coding sequence to the promoter: 12668112
cDNA nucleotide sequence (SEQ ID NO: 46):
ATAAAAACTTAATTAGTTTTTACAGAAGAAAAGAAAACAATGAGAGGTAAATTTCTAAGTTTA
CTGTTGCTCATTACTTTGGCCTGCATTGGAGTTTCCGCCAAGAAGCATTCCACAAGGCCTAGAT
TAAGAAGAAATGATTTCCCACAAGATTTCGTTTTTGGATCTGCTACTTCTGCTTATCAGTGTGA
AGGAGCTGCACATGAAGATGGTAGAGGTCCAAGTATCTGGGACTCCTTCTCTGAAAAATTCCC
AGAAAAGATAATGGATGGTAGTAATGGGTCCATTGCAGATGATTCTTACAATCTTTACAAGGA
AGATGTGAATTTGCTGCATCAAATTGGCTTCGATGCTTACCGATTTTCGATCTCATGGTCACGG
ATTTTGCCTCGTGGGACTCTAAAGGGAGGAATCAACCAGGCTGGAATTGAATATTATAACAAC
TTGATTAATCAACTTATATCTAAAGGAGTGAAGCCATTTGTCACACTCTTTCACTGGGACTTAC
CAGATGCACTCGAAAATGCTTACGGTGGCCTCCTTGGAGATGAATTTGTGAACGATTTCCGAG
ACTATGCAGAACTTTGTTTCCAGAAGTTTGGAGATAGAGTGAAGCAGTGGACGACACTAAACG
AGCCATATACAATGGTACATGAAGGTTATATAACAGGTCAAAAGGCACCTGGAAGATGTTCCA
ATTTCTATAAACCTGATTGCTTAGGTGGCGATGCAGCCACGGAGCCTTACATCGTCGGCCATA
ACCTCCTCCTTGCTCATGGAGTTGCCGTAAAAGTATATAGAGAAAAGTACCAGGCAACTCAGA
AAGGTGAAATTGGTATTGCCTTAAACACAGCATGGCACTACCCTTATTCAGATTCATATGCTG
ACCGGTTAGCTGCGACTCGAGCGACTGCCTTCACCTTCGACTACTTCATGGAGCCAATCGTGT
ACGGTAGATATCCAATTGAAATGGTCAGCCACGTTAAAGACGGTCGTCTTCCTACCTTCACAC
CAGAAGAGTCCGAAATGCTCAAAGGATCATATGATTTCATAGGCGTTAACTATTACTCATCTC
TTTACGCAAAAGACGTGCCGTGTGCAACTGAAAACATCACCATGACCACCGATTCTTGCGTCA
GCCTCGTAGGTGAACGAAATGGAGTGCCTATCGGTCCAGCGGCTGGATCGGATTGGCTTTTGA
TATATCCCAAGGGTATTCGTGATCTCCTACTACATGCAAAATTCAGATACAATGATCCCGTCTT
GTACATTACAGAGAATGGAGTGGATGAAGCAAATATTGGCAAAATATTTCTTAACGACGATTT
GAGAATTGATTACTATGCTCATCACCTCAAGATGGTTAGCGATGCTATCTCGATCGGGGTGAA
TGTGAAGGGATATTTCGCGTGGTCATTGATGGATAATTTCGAGTGGTCGGAAGGATACACGGT
CCGGTTCGGGCTAGTGTTTGTGGACTTTGAAGATGGACGTAAGAGGTATCTGAAGAAATCAGC
TAAGTGGTTTAGGAGATTGTTGAAGGGAGCGCATGGTGGGACGAATGAGCAGGTGGCTGTTA
TTTAATAAACCACGAGTCATTGGTCAATTTAGTCTACTGTTTCTTTTGCTCTATGTACAGAAAG
AAAATAAACTTTCCAAAATAAGAGGTGGCTTTGTTTGGACTTTGGATGTTACTATATATATTG
GTAATTCTTGGCGTTTGTTAGTTTCCAAACCAAACATTAAT
Coding sequence (SEQ ID NO: 47):
MRGKFLSLLLLITLACIGVSAKKHSTRPRLRRNDFPQDFVFGSATSAYQCEGAAHEDGRGPSIWDSF
SEKFPEKIMDGSNGSIADDSYNLYKEDVNLLHQIGFDAYRFSISWSRILPRGTLKGGINQAGIEYYN
NLINQLISKGVKPFVTLFHWDLPDALENAYGGLLGDEFVNDFRDYAELCFQKFGDRVKQWTTLNE
PYTMVHEGYITGQKAPGRCSNEYKPDCLGGDAATEPYIVGHNLLLAHGVAVKVYREKYQATQKG
EIGIALNTAWHYPYSDSYADRLAATRATAFTFDYFMEPIVYGRYPIEMVSHVKDGRLPTFTPEESE
MLKGSYDFIGVNYYSSLYAKDVPCATENITMTTDSCVSLVGERNGVPIGPAAGSDWLLIYPKGIRD
LLLHAKFRYNDPVLYITENGVDEANIGKIFLNDDLRIDYYAHHLKMVSDAISIGVNVKGYFAWSL
MDNFEWSEGYTVRFGLVFVDFEDGRKRYLKKSAKWFRRLLKGAHGGTNEQVAVI*
Promoter YP0244
Modulates the gene: Ca2+-ATPase 7
The GenBank description of the gene: NM_127860 Arabidopsis thaliana potential
calcium-transporting ATPase 7, plasma membrane-type (Ca2+-ATPase, isoform 7)
(At2g22950) mRNA, complete cds gi|18400128|ref|NM_127860.1|[18400128]
The promoter sequence (SEQ ID NO: 48):
5′aaagtcttatttgtgaaattttacaaatgttggaaaaaagcattttatggtgctatatttgtcaatttc
ccttgattatatatccttttgaaaagtaatgttttttttatgtgtgtgtattcatgaaccttggaaaaact
acaaatcagatcatggtttgttttaggtgaaaaatttagaacacagttacgcaagaaagatatcggtaaat
ttttgtttctttgaatcgaaattaatcaaaaagtattttccattatataacaacaactaatctctgttttt
tttttttttttttaacaactaatctcttatcaaaatgacactacagaatcacgattgtaaatctttaaaag
gcagtctgaaaaatattcatgaggatgagattttattcattcatggttgtaagtaatcattatgtaaagtt
taggataaggacgttcaaaatcatataaaaaaactctacgaataaagtttatagtctatcatattgattca
tatttcatagaaagttactggaaaacattacacaagtattctcgatttttacgagtttgtttagtagtcgc
aaaattttattttacttttgagtatacgaacccataagctgattttctttccaagttccaataatgatatc
atagtgtactcttcatgaatgtttcaagcatataattataacgttcataagtaatattctactgcatgttt
gttatTATAaattaactaataatcgaacgtatgagttttgattgagattgttgtgctcacgaaatgaagga
ctcggtcaattctaaagcttaaaataagaagctcagatcttaaaactcgctttcgtcttcgtcctccattt
aagtttgcgattcttttgctcttctttctctctcacatttttgtcccaaaacaataaaaagaaacaataat
agaaagtgttacagaaaaagaaagaaaac 3′-ATG
The promoter was cloned from the organism: Arabidopsis thaliana, WS ecotype

Alternative nucleotides:
Predicted (Columbia)
Experimental (Wassilewskija)

| | | |
|---|---|---|
| Sequence Position (bp) | Mismatch | Columbia/Wassilewskija |

| | | |
|---|---|---|
| 90 | SNP | a/g |
| 183 | SNP | t/c |
| 373 | SNP | t/c |
| 380 | No g in Ws | -/- |
| 393 | No a in Ws | -/- |
| 717 | SNP | t/c |
| 774 | SNP | a/g |

The promoter was cloned in the vector: pNewbin4-HAP1-GFP
When cloned into the vector the promoter was operably linked to a marker, which was
the type: GFP-ER
Promoter-marker vector was tested in: Arabidopsis thaliana, WS ecotype
Generation screened: XT1 Mature XT2 Seedling T2 Mature T3 Seedling
The spatial expression of the promoter-marker vector was found observed in and would
be useful in expression in any or all of the following:

| | |
|---|---|
| Flower | H pollen |

Observed expression pattern of the promoter-marker vector was in:
T1 mature: Pollen specific expression in mature plants.
T2 seedling: No GFP expression observed.
The promoter can be of use in the following trait and sub-trait areas: (search for
the trait and subtrait table)

| | |
|---|---|
| Trait Area: | Paternal inheritance trait where 50% is desired |
| Sub-trait Area: | Yield |

The promoter has utility in:

| | |
|---|---|
| Utility: | Modulation of pollen tube growth, incompatibility |

| | | |
|---|---|---|
| Misc. promoter information: Bidirectionality: Pass | Exons: Pass | Repeats: No |

The Ceres cDNA ID of the endogenous coding sequence to the promoter: 12736016
cDNA nucleotide sequence (SEQ ID NO: 49):
atggagagttacctcaactcgaatttcgacgttaaggcgaagcattcgtcggaggaagtgctagaaaaatg
gcggaatctttgcagtgtcgtcaagaacccgaaacgtcggtttcgattcactgccaatctctccaaacgtt
acgaagctgctgccatgcgccgcaccaaccaggagaaattaaggattgcagttctcgtgtcaaaagccgca
tttcaatttatctctggtgtttctccaagtgactacaaggtgcctgaggaagttaaagcagcaggctttga
catttgtgcagacgagttaggatcaatagtggaaggtcatgatgtgaagaagctcaagttccatggtggtg
ttgatggtctttcaggtaagctcaaggcatgtcccaatgctggtctctcaacaggtgaacctgagcagtta
agcaaacgacaagagcttttcggaatcaataagtttgcagagagtgaattacgaagtttctgggtgtttgt
ttgggaagcacttcaagatatgactcttatgattcttggtgtttgtgctttcgtctctttgattgttggga
ttgcaactgaaggatggcctcaaggatcgcatgatggtcttggcattgttgctagtattcttttagttgtg
tttgtgacagcaactagtgactatagacaatctttgcagttccgggatttggataaagagaagaagaagat
cacggttcaagttacgcgaaacgggtttagacaaaagatgtctatatatgatttgctccctggagatgttg
ttcatcttgctatcggagatcaagtccctgcagatggtcttttcctctcgggattctctgttgttatcgat
gaatcgagtttaactggagagagtgagcctgtgatggtgactgcacagaaccctttccttctctctggaac
caaagttcaagatgggtcatgtaagatgttggttacaacagttgggatgagaactcaatggggaaagttaa
tggcaacacttagtgaaggaggagatgacgaaactccgttgcaggtgaaacttaatggagttgcaaccatc
attgggaaaattggtctttccttcgctattgttacctttgcggttttggtacaaggaatgtttatgaggaa
gctttcattaggccctcattggtggtggtccggagatgatgcattagagcttttggagtattttgctattg
ctgtcacaattgttgttgttgcggttcctgaaggtttaccattagctgtcacacttagtctcgcgtttgcg
atgaagaagatgatgaacgataaagcgcttgttcgccatttagcagcttgtgagacaatgggatctgcaac
taccatttgtagtgacaagactggtacattaacaacaaatcacatgactgttgtgaaatcttgcatttgta
tgaatgttcaagatgtagctagcaaaagttctagtttacaatctgatatccctgaagctgccttgaaacta
cttctccagttgatttttaataataccggtggagaagttgttgtgaacgaacgtggcaagactgagatatt
ggggacaccaacagagactgctatattggagttaggactatctcttggaggtaagtttcaagaagagagac
aatctaacaaagttattaaagttgagccttttaactcaacaaagaaaagaatgggagtagtcattgagctg
cctgaaggaggacgcattcgcgctcacacgaaaggagcttcagagatagttttagcggcttgtgataaagt
catcaactcaagtggtgaagttgttccgcttgatgatgaatccatcaagttcttgaatgttacaatcgatg
agtttgcaaatgaagctcttcgtactctttgccttgcttatatggatatcgaaagcgggttttcggctgat
gaaggtattccggaaaaagggtttacatgcatagggattgttggtatcaaagaccctgttcgtcctggagt
tcgggagtccgtggaactttgtcgccgtgcgggtattatggtgagaatggttacaggagataacattaaca
ccgcaaaggctattgctagagaatgtggaattctcactgatgatggtatagcaattgaaggtcctgtgttt
agagagaagaaccaagaagagatgcttgaactcattcccaagattcaggtcatggctcgttcttccccaat
ggacaagcatacactggtgaagcagttgaggactacttttgatgaagttgttgctgtgactggcgacggga
caaacgatgcaccagcgctccacgaggctgacataggattagcaatgggcattgccgggactgaagtagcg
aaagagattgcggatgtcatcattctcgacgataacttcagcacaatcgtcaccgtagcgaaatggggacg
ttctgtttacattaacattcagaaatttgtgcagtttcaactaacagtcaatgttgttgcccttattgtta
acttctcttcagcttgcttgactggaagtgctcctctaactgctgttcaactgctttgggttaacatgatc
atggacacacttggagctcttgctctagctacagaacctccgaacaacgagctgatgaaacgtatgcctgt
tggaagaagagggaatttcattaccaatgcgatgtggagaaacatcttaggacaagctgtgtatcaattta
ttatcatatggattctacaggccaaagggaagtccatgtttggtcttgttggttctgactctactctcgta
ttgaacacacttatcttcaactgctttgtattctgccaggttttcaatgaagtaagctcgcgggagatgga
agagatcgatgttttcaaaggcatactcgacaactatgttttcgtggttgttattggtgcaacagttttct
ttcagatcataatcattgagttcttgggcacatttgcaagcaccacacctcttacaatagttcaatggttc
ttcagcattttcgttggcttcttgggtatgccgatcgctgctggcttgaagaaaatacccgtgtga
Coding sequence (SEQ ID NO: 50):
MESYLNSNFDVKAKHSSEEVLEKWRNLCSVVKNPKRRFRFTANLSKRYEAAAMRRTNQEKLRIA
VLVSKAAFQFISGVSPSDYKVPEEVKAAGFDICADELGSIVEGHDVKKLKFHGGVDGLSGKLKACP
NAGLSTGEPEQLSKRQELFGINKFAESELRSFWVFVWEALQDMTLMILGVCAFVSLIVGIATEGWP
QGSHDGLGIVASILLVVFVTATSDYRQSLQFRDLDKEKKKITVQVTRNGFRQKMSIYDLLPGDVVH
LAIGDQVPADGLFLSGFSVVIDESSLTGESEPVMVTAQNPFLLSGTKVQDGSCKMLVTTVGMRTQ
WGKLMATLSEGGDDETPLQVKLNGVATIIGKIGLSFAIVTFAVLVQGMFMRKLSLGPHWWWSGD
DALELLEYFAIAVTIVVVAVPEGLPLAVTLSLAFAMKKMMNDKALVRHLAACETMGSATTICSDK
TGTLTTNHMTVVKSCICMNVQDVASKSSSLQSDIPEAALKLLLQLIFNNTGGEVVVNERGKTEILG
TPTETAILELGLSLGGKFQEERQSNKVIKVEPFNSTKKRMGVVIELPEGGRIRAHTKGASEIVLAAC
DKVINSSGEVVPLDDESIKFLNVTIDEFANEALRTLCLAYMDIESGFSADEGIPEKGFTCIGIVGIKDP
VRPGVRESVELCRRAGIMVRMVTGDNINTAKAIARECGILTDDGIAIEGPVFREKNQEEMLELIPKI
QVMARSSPMDKHTLVKQLRTTFDEVVAVTGDGTNDAPALHEADIGLAMGIAGTEVAKEIADVIIL
DDNFSTIVTVAKWGRSVYINIQKFVQFQLTVNVVALIVNESSACLTGSAPLTAVQLLWVNMIMDTL
GALALATEPPNNELMKRMPVGRRGNFITNAMWRNILGQAVYQFIIIWILQAKGKSMFGLVGSDST
LVLNTLIFNCFVFCQVFNEVSSREMEEIDVFKGILDNYVFVVVIGATVFFQIIIIEFLGTFASTTPLTIV
QWFFSIFVGFLGMPIAAGLKKIPV*
Promoter YP0226
Modulates the gene: Indoleacetic acid-induced protein 12
The GenBank description of the gene: NM_100334 Arabidopsis thaliana auxin-responsive
protein IAA12 (Indoleacetic acid-induced protein 12) (At1g04550) mRNA, complete cds
gi|30678909|ref|NM_100334.2
The promoter sequence (SEQ ID NO: 51):
5′tcaaaagtgtaatttccacaaaccaattgcgcctgcaaaagttttcaaaggatcatcaaacataatgat
gaatatctcatcaccacgattttataataatgcatcttttcccaccattttttttccctcactttctttta
taatcttgttcgacaacaatcatggtctaaggaaaaagttgaaaatatatattatcttagttattagaaaa
gaaagataatcaaatggtcaatatgcaaatggcatatgaccataaacgagtttgctagtataaagaatgat
ggccaacctgttaaagagagactaaaattaggtctaaaatctaggagcaatgtaaccaatacatagtatat
gaaatataaaagttaatttagattttttgattagcccaaattaaagaaaaatggtatttaaaacagagact
cttcatcctaaaggctaaagcaatacaatttttggttaagaaaagaaaaaaaccacaagcggaaaagaaaa
caaaaaagaactatattatgatgcaacagcaacacaaagcaaaaccttgcacacacacatacaactgtaaa
caagtttcttgggactctctattttctcttgctgcttgaaccaaacacaacaacgatatcccaacgagagc
acaacaggtttgattatgtcggaagacaagttttgagagaaaacaaacaatatttTATAacaaaggagaag
acttttggttagaaaaaattggtatggccattacaagacatatgggtcccaattctcatcactctctccac
caccaaaatcctcctctctctctctctcttttactctgttttcatcatctctttctctcgtctctctcaaa
ccctaaatacactctttctcttcttgttgtctccattctctctgtgtcatcaagcttcttttttgtgtggg
ttatttgaaagacactttctctgctggtatcattggagt 3′-ATG
The promoter was cloned from the organism: Arabidopsis thaliana, WS ecotype

Alternative nucleotides:

| | | |
|---|---|---|
| Sequence (bp) | Mismatch | Columbia/Wassilewskija |

| | | |
|---|---|---|
| 523 | SNP | g/- |
| 558 | SNP | a/c |
| 741 | SNP | a/g |

The promoter was cloned in the vector: pNewbin4-HAP1-GFP
When cloned into the vector the promoter was operably linked to a marker, which was
the type: GFP-ER
Promoter-marker vector was tested in: Arabidopsis thaliana, WS ecotype
Generation screened: XT1 Mature XT2 Seedling T2 Mature T3 Seedling
The spatial expression of the promoter-marker vector was found observed in and would
be useful in expression in any or all of the following:

| | |
|---|---|
| Flower | M vascular |
| Silique | M placenta, M vascular |
| Hypocotyl | H vascular |
| Cotyledon | H vascular, H petiole |
| Primary Root | H vascular |

Observed expression pattern of the promoter-marker vector was in:
T1 mature: GFP expressed in vasculature of silique and pedicles of flowers.
T2 seedling: High GFP expression throughout vasculature of root, hypocotyl, and
petioles.

| | | |
|---|---|---|
| Misc. promoter information: Bidirectionality: Pass | Exons: Pass | Repeats: No |

Optional Promoter Fragments: 5′ UTR region at base pairs 832-1000
The Ceres cDNA ID of the endogenous coding sequence to the promoter: 12327003
cDNA nucleotide sequence (SEQ ID NO: 52):
ACTCTGTTTTCATCATCTCTTTCTCTCGTCTCTCTCAAACCCTAAATACACTCTTTCTCTTCTTG
TTGTCTCCATTCTCTCTGTGTCATCAAGCTTCTTTTTTGTGTGGGTTATTTGAAAGACACTTTCT
CTGCTGGTATCATTGGAGTCTAGGGTTTTGTTATTGACATGCGTGGTGTGTCAGAATTGGAGG
TGGGGAAGAGTAATCTTCCGGCGGAGAGTGAGCTGGAATTGGGATTAGGGCTCAGCCTCGGT
GGTGGCGCGTGGAAAGAGCGTGGGAGGATTCTTACTGCTAAGGATTTTCCTTCCGTTGGGTCT
AAACGCTCTGCTGAATCTTCCTCTCACCAAGGAGCTTCTCCTCCTCGTTCAAGTCAAGTGGTAG
GATGGCCACCAATTGGGTTACACAGGATGAACAGTTTGGTTAATAACCAAGCTATGAAGGCAG
CAAGAGCGGAAGAAGGAGACGGGGAGAAGAAAGTTGTGAAGAATGATGAGCTCAAAGATGT
GTCAATGAAGGTGAATCCGAAAGTTCAGGGCTTAGGGTTTGTTAAGGTGAATATGGATGGAGT
TGGTATAGGCAGAAAAGTGGATATGAGAGCTCATTCGTCTTACGAAAACTTGGCTCAGACGCT
TGAGGAAATGTTCTTTGGAATGACAGGTACTACTTGTCGAGAAAAGGTTAAACCTTTAAGGCT
TTTAGATGGATCATCAGACTTTGTACTCACTTATGAAGATAAGGAAGGGGATTGGATGCTTGT
TGGAGATGTTCCATGGAGAATGTTTATCAACTCGGTGAAAAGGCTTCGGATCATGGGAACCTC
AGAAGCTAGTGGACTAGCTCCAAGACGTCAAGAGCAGAAGGATAGACAAAGAAACAACCCTG
TTTAGCTTCCCTTCCAAAGCTGGCATTGTTTATGTATTGTTTGAGGTTTGCAATTTACTCGATA
CTTTTTGAAGAAAGTATTTTGGAGAATATGGATAAAAGCATGCAGAAGCTTAGATATGATTTG
AATCCGGTTTTCGGATATGGTTTTGCTTAGGTCATTCAATTCGTAGTTTTCCAGTTTGTTTCTTC
TTTGGCTGTGTACCAATTATCTATGTTCTGTGAGAGAAAGCTCTTGTTTATTTGTTCTCTCAGA
TTGTAAATAGTTGAAGTTATCTAATTAATGTGATAAGAGTTATGTTTATGATTCC
Coding sequence (SEQ ID NO: 53):
MRGVSELEVGKSNLPAESELELGLGLSLGGGAWKERGRILTAKDFPSVGSKRSAESSSHQGASPPR
SSQVVGWPPIGLHRMNSLVNNQAMKAARAEEGDGEKKVVKNDELKDVSMKVNPKVQGLGFVK
VNMDGVGIGRKVDMRAHSSYENLAQTLEEMFFGMTGTTCREKVKPLRLLDGSSDFVLTYEDKEG
DWMLVGDVPWRMFINSVKRLRIMGTSEASGLAPRRQEQKDRQRNNPV*
Promoter PT0511
Modulates the gene: Major intrinsic protein (MIP)
The GenBank description of the gene: : NM_106724 Arabidopsis thaliana major intrinsic
protein (MIP) family (At1g80760) mRNA, complete cds
gi|30699534|ref|NM_106724.2|[30699534].
The promoter sequence (SEQ ID NO: 54):
5′gacgggtcatcacagattcttcgtttttttatagatagaaaaggaataacgttaaaagtatacaaatta
tatgcaagagtcattcgaaagaattaaataaagagatgaactcaaaagtgattttaaattttaatgataag
aatatacatctcacagaaatcttttatttgacatgtaaaatcttgttttcacctatcttttgttagtaaac
aagaatatttaatttgagcctcacttggaacgtgataataatatacatcttatcataattgcatattttgc
ggatagtttttgcatggggagattaaaggcttaataaagccttgaatttccgaggggaggaatcatgtttt
atacttgcaaactatacaaccatctgcatcgataattggtgttaatacatgcaaggattatacactaaaac
aaatcatttatttccttacaaaaagagagtcgactgtgagtcacattctgtgacaaggaaaggtcaagaac
catcgcttttatcatcattctctttgctaacaacttacaaccacacaaacgcaagagttccattctcatgg
agaagaacatattatgcaaaataatgtatgtcgatcgatagagaaaaggatccacaattattgctccatct
caaaagcttctttagtacacgatacatgtatcatgtaaatagaaatatgaaagatacaatacacgacccat
tctcataaagatagcaacatttcatgttatgtaaagagtcttccttaggacacatgcattaaaactaagga
ttaccaacccacttactcctcactccaaccaaatatcaatcatctattttgggtccttcactcataagtca
actctcatgccttcctctataaataccgtaccctacgcatcccttagttctacatcacataaaaacaatca
tagcaaaaacaTATAtcctcaaattaatt 3′-cATG
The promoter was cloned from the organism: Arabidopsis thaliana, Columbia ecotype

Alternative nucleotides:

| Predicted Position (bp) | Mismatch | Predicted/Experimental |
|---|---|---|
| 1-1000 | None | Identities = 1000/1000 (100%) |

The promoter was cloned in the vector: pNewbin4-HAP1-GFP
When cloned into the vector the promoter was operably linked to a marker, which was
the type: GFP-ER
Promoter-marker vector was tested in: Arabidopsis thaliana, WS ecotype
Generation screened: XT1 Mature XT2 Seedling T2 Mature T3 Seedling
The spatial expression of the promoter-marker vector was found observed in and would
be useful in expression in any or all of the following:

| | |
|---|---|
| Flower | H filament H anther L vascular |
| Cotyledon | L vascular L petiole |
| Primary Root | L epidermis |

Observed expression pattern of the promoter-marker vector was in:
T1 mature: High expression at vascular connective tissue between locules of anther.
T2 seedling: Low expression in root epidermal cells and vasculature of petioles.

| | | |
|---|---|---|
| Misc. promoter information: Bidirectionality: Pass | Exons: Pass | Repeats: No |

Optional Promoter Fragments: 5′ UTR region at base pairs 927-1000.
The Ceres cDNA ID of the endogenous coding sequence to the promoter: 12711931
cDNA nucleotide sequence (SEQ ID NO: 55):
ATGGATCATGAGGAAATTCCATCCACGCCCTCAACGCCGGCGACAACCCCGGGGACTCCAGGA
GCGCCGCTCTTTGGAGGATTCGAAGGGAAGAGGAATGGACACAATGGTAGATACACACCAAA
GTCACTTCTCAAAAGCTGCAAATGTTTCAGTGTTGACAATGAATGGGCTCTTGAAGATGGAAG
ACTCCCTCCGGTCACTTGCTCTCTCCCTCCCCCTAACGTTTCCCTCTACCGCAAGTTGGGAGCA
GAGTTTGTTGGGACATTGATCCTGATATTCGCCGGAACAGCGACGGCGATCGTGAACCAGAAG
ACAGATGGAGCTGAGACGCTTATTGGTTGCGCCGCCTCGGCTGGTTTGGCGGTTATGATCGTT
ATATTATCGACCGGTCACATCTCCGGGGCACATCTCAATCCGGCTGTAACCATTGCCTTTGCTG
CTCTCAAACACTTCCCTTGGAAACACGTGCCGGTGTATATCGGAGCTCAGGTGATGGCCTCCG
TGAGTGCGGCGTTTGCACTGAAAGCAGTGTTTGAACCAACGATGAGCGGTGGCGTGACGGTG
CCGACGGTGGGTCTCAGCCAAGCTTTCGCCTTGGAATTCATTATCAGCTTCAACCTCATGTTCG
TTGTCACAGCCGTAGCCACCGACACGAGAGCTGTGGGAGAGTTGGCGGGAATTGCCGTAGGA
GCAACGGTCATGCTTAACATACTTATAGCTGGACCTGCAACTTCTGCTTCGATGAACCCTGTAA
GAACACTGGGTCCAGCCATTGCAGCAAACAATTACAGAGCTATTTGGGTTTACCTCACTGCCC
CCATTCTTGGAGCGTTAATCGGAGCAGGTACATACACAATTGTCAAGTTGCCAGAGGAAGATG
AAGCACCCAAAGAGAGGAGGAGCTTCAGAAGATGA
Coding sequence (SEQ ID NO: 56):
MDHEEIPSTPSTPATTPGTPGAPLFGGFEGKRNGHNGRYTPKSLLKSCKCFSVDNEWALEDGRLPP
VTCSLPPPNVSLYRKLGAEFVGTLILIFAGTATAIVNQKTDGAETLIGCAASAGLAVMIVILSTGHIS
GAHLNPAVTIAFAALKHFPWKHVPVYIGAQVMASVSAAFALKAVFEPTMSGGVTVPTVGLSQAF
ALEFIISFNLMFVVTAVATDTRAVGELAGIAVGATVMLNILIAGPATSASMNPVRTLGPAIAANNYR
AIWVYLTAPILGALIGAGTYTIVKLPEEDEAPKERRSFRR*
Promoter PT0506
Modulates the gene: CYCD1
The GenBank description of the gene: NM_105689 Arabidopsis thaliana cyclin delta-1
(CYCD1) (At1g70210) mRNA, complete cds gi|30698007|ref|NM_105689.2|[30698007]. Go
function: cyclin-dependent protein kinase regulator.
The promoter sequence (SEQ ID NO: 57):

(SEQ ID NO: 58)

5′cgctccagaccactgtttgctttcctctgattaaccaatctcaattaaactactaatttataattcaag
ataattagataaccaatcttaaaatttggaatcttcttccctcacttgatattacaaaaaaaaaactgatt
tatcatacggttaattcaagaaaacagcaaaaaaattgcactataatgcaaaacatcaattaattacattc
gattaaaaaatcatcattgaatctaaaatggcctcaaatctattgagcatttgtcatgtgcctaaaatggt
tcaggagttttacatctaatcacataaaaagcaaacaataaccaaaaaaattgcattttagcaaatcaaat
acttatatatatacgtatgattaagcgtcatgactttaaaacctctgtaaaattttgatttatttttcgat
gcttttattttttaaccaatagtaataaagtccaaatcttaaatacgaaaaaatgtttctttctaagcgac
caacaaaatggtccaaatcacagaaaatgttccataatccaggcccattaagctaatcaccaagtaataca
ttacacgtcaccaattaatacattacacgtacggccttctctcttcacgagtaatatgcaaacaaacgtac
attagctgtaatgtactcactcatgcaacgtcttaacctgccacgtattacgtaattacaccactccttgt
tcctaacctacgcatttcactttagcgcatgttagtcaaaaaacacaaacataaactacaaataaaaaaac
tcaaaacaaaacccaatgaacgaacggaccagccccgtctcgattgatggaacagtgacaacagtcccgtt
ttctcgggcataacggaaacggtaaccgtctctctgtttcatttgcaacaacaccattttTATAaataaaa
acacatttaaataaaaaattattaaaacc 3′-
tatatccaaacaaatgaatgtgttaaaccttcactcttctctccacacaaaattcaaaaacctcacatttc
acttctctcttctcgcttcttctagatctcaccggtttatctagctccggtttgattcatctccggttatg
gggagagaATG
The promoter was cloned from the organism: Arabidopsis thaliana, Columbia ecotype

Alternative nucleotides:

| | | |
|---|---|---|
| Predicted Position (bp) | Mismatch | Predicted/Experimental |

| | | |
|---|---|---|
| 1-1000 | None | Identities = 1000/1000 (100%) |

The promoter was cloned in the vector: pNewbin4-HAP1-GFP
When cloned into the vector the promoter was operably linked to a marker, which was
the type: GFP-ER
Promoter-marker vector was tested in: Arabidopsis thaliana, WS ecotype
Generation screened: XT1 Mature XT2 Seedling T2 Mature T3 Seedling
The spatial expression of the promoter-marker vector was found observed in and would
be useful in expression in any or all of the following:

| | |
|---|---|
| Flower | L anther |

Observed expression pattern of the promoter-marker vector was in:
T1 mature: Low expression in anther walls early in stamen development through pre-
dehiscence stage. Not in pollen
T2 seedling: No expression observed.

| | | |
|---|---|---|
| Misc. promoter information: Bidirectionality: Pass | Exons: Pass | Repeats: No |

The Ceres cDNA ID of the endogenous coding sequence to the promoter: 13497447
cDNA nucleotide sequence (SEQ ID NO: 59):
ATATATCCAAACAAATGAATGTGTTAAACCTTCACTCTTCTCTCCACACAAAATTCAAAAACCT
CACATTTCACTTCTCTCTTCTCGCTTCTTCTAGATCTCACCGGTTTATCTAGCTCCGGTTTGATT
CATCTCCGGTTATGGGGAGAGAATGAGGAGTTACCGTTTTAGTGATTATCTACACATGTCTGT
TTCATTCTCTAACGATATGGATTTGTTTTGTGGAGAAGACTCCGGTGTGTTTTCCGGTGAGTCA
ACGGTTGATTTCTCGTCTTCCGAGGTTGATTCATGGCCTGGTGATTCTATCGCTTGTTTTATCG
AAGACGAGCGTCACTTCGTTCCTGGACATGATTATCTCTCTAGATTTCAAACTCGATCTCTCGA
TGCTTCCGCTAGAGAAGATTCCGTCGCATGGATTCTCAAGGTACAAGCGTATTATAACTTTCA
GCCTTTAACGGCGTACCTCGCCGTTAACTATATGGATCGGTTTCTTTACGCTCGTCGATTACCG
GAAACGAGTGGTTGGCCAATGCAACTTTTAGCAGTGGCATGCTTGTCTTTAGCTGCAAAGATG
GAGGAAATTCTCGTTCCTTCTCTTTTTGATTTTCAGGTTGCAGGAGTGAAGTATTTATTTGAAG
CAAAAACTATAAAAAGAATGGAACTTCTTGTTCTAAGTGTGTTAGATTGGAGACTAAGATCGG
TTACACCGTTTGATTTCATTAGCTTCTTTGCTTACAAGATCGATCCTTCGGGTACCTTTCTCGG
GTTCTTTATCTCCCATGCTACAGAGATTATACTCTCCAACATAAAAGAAGCGAGCTTTCTTGAG
TACTGGCCATCGAGTATAGCTGCAGCCGCGATTCTCTGTGTAGCGAACGAGTTACCTTCTCTAT
CCTCTGTTGTCAATCCCCACGAGAGCCCTGAGACTTGGTGTGACGGATTGAGCAAAGAGAAGA
TAGTGAGATGCTATAGACTGATGAAAGCGATGGCCATCGAGAATAACCGGTTAAATACACCA
AAAGTGATAGCAAAGCTTCGAGTGAGTGTAAGGGCATCATCGACGTTAACAAGGCCAAGTGA
TGAATCCTCTTTCTCATCCTCTTCTCCTTGTAAAAGGAGAAAATTAAGTGGCTATTCATGGGTA
GGTGATGAAACATCTACCTCTAATTAAAATTTGGGGAGTGAAAGTAGAGGACCAAGGAAACA
AAACCTAGAAGAAAAAAAACCCTCTTCTGTTTAAGTAGAGTATATTTTTTAACAAGTACATAG
TAATAAGGGAGTGATGAAGAAAAGTAAAAGTGTTTATTGGCTGAGTTAAAGTAATTAAGAGT
TTTCCAACCAAGGGGAAGGAATAAGAGTTTTGGTTACAATTTCTTTTATGGAAAGGGTAAAAA
TTGGGTTTTGGGGTTGGTTGGTTGGTTGGGAGAGACGAAGCTCATCATTAATGGCTTTGCAGA
TTCCCAAGAAAGCAAAATGAGTAAGTGAGTGTAACACACACGTGTTAGAGAAAAGATATGAT
CATGTGAGTGTGTGTGTGTGAGAGAGAGAGAGAAGAGTATTTGCATTAGAGTCCTCATCACAC
AGGTACTGATGGATAAGACAGGGGAGCGTTTGCAAAAGATTTGTGAGTGGAGATTTTTCTGAG
CTCTTTGTCTTAATGGATCGCAGCAGTTCATGGGACCCTTCCTCAGCTTCATCATCAAACAAAA
AAAAAATCAAGTTGCGAAGTATATATAATTTGTTTTTTTGTTTGGATTTTTAAGATTTTTGATT
CCTTGTGTGTGACTTCACGTGACGGAGGCGTGTGTCTCACGTGTTTGTTTTCTCTTCAAATCTT
TTATTTTGGCGGGAAATTTTGTGTTTTTGATTTCTACGTATTCGTGGACTCCAAATGAGTTTTG
TCACGGTGCGTTTTAGTAGCGTTTGCATGCGTGTAAGGTGTCACGTATGTGTATATATATGATT
TTTTTTTGGTTTCTTGAAAGGTTGAATTTTATAAATAAAACGTTTCTATTAT
Coding sequence (SEQ ID NO: 60):
MRSYRFSDYLHMSVSFSNDMDLFCGEDSGVFSGESTVDFSSSEVDSWPGDSIACFIEDERHFVPGH
DYLSRFQTRSLDASAREDSVAWILKVQAYYNEQPLTAYLAVNYMDRFLYARRLPETSGWPMQLL
AVACLSLAAKMEEILVPSLFDFQVAGVKYLFEAKTIKRMELLVLSVLDWRLRSVTPFDFISFFAYKI
DPSGTFLGFFISHATEIILSNIKEASFLEYWPSSIAAAAILCVANELPSLSSVVNPHESPETWCDGLSK
EKIVRCYRLMKAMAIENNRLNTPKVIAKLRVSVRASSTLTRPSDESSFSSSSPCKRRKLSGYSWVG
DETSTSN*
Promoter YP0377
Modulates the gene: product = “glycine-rich protein”, note: unknown protein
The GenBank description of the gene: : NM_100587 Arabidopsis thaliana glycine-rich
protein (At1g07135) mRNA, complete cds gi|22329385|ref|NM_100587.2|[22329385]
The promoter sequence (SEQ ID NO: 61):
5′tttaaacataacaatgaattgcttggatttcaaactttattaaatttggattttaaattttaatttgat
tgaattatacccccttaattggataaattcaaatatgtcaactttttttttttgtaagatttttttatgga
aaaaaaaattgattattcactaaaaagatgacaggttacttataatttaatatatgtaaaccctaaaaaga
agaaaatagtttctgttttcactttaggtcttattatctaaacttctttaagaaaatcgcaataaattggt
ttgagttctaactttaaacacattaatatttgtgtgctatttaaaaaataatttacaaaaaaaaaaacaaa
ttgacagaaaatatcaggttttgtaataagatatttcctgataaatatttagggaatataacatatcaaaa
gattcaaattctgaaaatcaagaatggtagacatgtgaaagttgtcatcaatatggtccacttttctttgc
tctataacccaaaattgaccctgacagtcaacttgtacacgcggccaaacctttttataatcatgctattt
atttccttcatttttattctatttgctatctaactgatttttcattaacatgataccagaaatgaatttag
atggattaattcttttccatccacgacatctggaaacacttatctcctaattaaccttactttttttttag
tttgtgtgctccttcataaaatctatattgtttaaaacaaaggtcaataaatataaatatggataagtata
ataaatctttattggatatttctttttttaaaaaagaaataaatcttttttggatattttcgtggcagcat
cataatgagagactacgtcgaaactgctggcaaccacttttgccgcgtttaatttctttctgaggcttata
taaatagatcaaaggggaaagtgagaTAT 3′
The promoter was cloned from the organism: Arabidopsis thaliana, Columbia ecotype

Alternative nucleotides:

| | | |
|---|---|---|
| Predicted Position (bp) | Mismatch | Predicted/Experimental |

| | | |
|---|---|---|
| 145 | Sequence or PCR error | ctttttttttttg/ |
| | | ctttttttt-ttg Exp.1 |
| | | ctttttttt--tg Exp.2 |

The promoter was cloned in the vector: pNewbin4-HAP1-GFP
When cloned into the vector the promoter was operably linked to a marker, which was
the type: GFP-ER
Promoter-marker vector was tested in: Arabidopsis thaliana, WS ecotype
Generation screened: XT1 Mature XT2 Seedling T2 Mature T3 Seedling
The spatial expression of the promoter-marker vector was found observed in and would
be useful in expression in any or all of the following:

| | |
|---|---|
| Flower | M sepal M petal M epidermis |
| Hypocotyl | L epidermis L vascular H stomata |
| Cotyledon | M vascular L epidermis |
| Primary Root | M epidermis M vascular M root hairs |

Observed expression pattern of the promoter-marker vector was in:
T1 mature: Expressed in epidermal cells of sepals and petals in developing flowers.
T2 seedling: Medium to low expression in epidermal and vascular cells of hypocotyls
and cotyledons. Epidermal and vascular expression at root transition zone decreasing
toward root tip.

| | | |
|---|---|---|
| Misc. promoter information: Bidirectionality: Pass | Exons: Pass | Repeats: No |

The Ceres cDNA ID of the endogenous coding sequence to the promoter: 13613778
cDNA nucleotide sequence (SEQ ID NO: 62):
AAAGAAAATGGGTTTGAGAAGAACATGGTTGGTTTTGTACATTCTCTTCATCTTTCATCTTCAG
CACAATCTTCCTTCCGTGAGCTCACGACCTTCCTCAGTCGATACAAACCACGAGACTCTCCCTT
TTAGTGTTTCAAAGCCAGACGTTGTTGTGTTTGAAGGAAAGGCTCGGGAATTAGCTGTCGTTA
TCAAAAAAGGAGGAGGTGGAGGAGGTGGAGGACGCGGAGGCGGTGGAGCACGAAGCGGCGG
TAGGAGCAGGGGAGGAGGAGGTGGCAGCAGTAGTAGCCGCAGCCGTGACTGGAAACGCGGC
GGAGGGGTGGTTCCGATTCATACGGGTGGTGGTAATGGCAGTCTGGGTGGTGGATCGGCAGG
ATCACATAGATCAAGCGGCAGCATGAATCTTCGAGGAACAATGTGTGCGGTCTGTTGGTTGGC
TTTATCGGTTTTAGCCGGTTTAGTCTTGGTTCAGTAGGGTTCAGAGTAATTATTGGCCATTTAT
TTATTGGTTTTGTAACGTTTATGTTTGTGGTCCGGTCTGATATTTATTTGGGCAAACGGTACAT
TAAGGTGTAGACTGTTAATATTATATGTAGAAAGAGATTCTTAGCAGGATTCTACTGGTAGTA
TTAAGAGTGAGTTATCTTTAGTATGCCATTTGTAAATGGAAATTTAATGAAATAAGAAATTGT
GAAATTTAAAC
Coding sequence (SEQ ID NO: 63):
KKMGLRRTWLVLYILFIFHLQHNLPSVSSRPSSVDTNHETLPFSVSKPDVVVFEGKARELAVVI
KKGGGGGGGGRGGGGARSGGRSRGGGGGSSSSRSRDWKRGGGVVPIHTGGGNGSLGGGS
AGSHRSSGSMNLRGTMCAVCWLALSVLAGLVLVQ*

TABLE 2
Summary of Promoter Expression Results

| Promoter | Relvant Plant Tissue/Organ | | | | | | | | |
|---|---|---|---|---|---|---|---|---|---|
| Name | Fl | Si | Lf | St | Em | Ov | Hy | Co | Rt |
| YP0226 | Y | Y | | | | | Y | Y | Y |
| YP0244 | Y | | | | | | | | |
| YP0286 | Y | | | Y | | | Y | Y | Y |
| YP0289 | Y | | | | | Y | | Y | Y |
| YP0356 | Y | Y | | Y | | Y | Y | Y | |
| YP0374 | | | | | | Y | Y | | Y |
| YP0377 | Y | | | | | | Y | Y | Y |
| YP0380 | Y | Y | Y | Y | | | Y | Y | Y |
| YP0381 | Y | | | | | | Y | | Y |
| YP0382 | Y | | | | | | | | Y |
| YP0388 | Y | Y | Y | | | Y | | | Y |
| YP0396 | Y | Y | Y | | | Y | | | Y |
| PT0506 | Y | | | | | | | | |
| PT0511 | Y | | | | | | | Y | Y |
| YP0275 | | | | | | | | | Y |
| YP0337 | | | | | | | | | Y |
| YP0384 | | | | | | | | | Y |
| YP0385 | Y | Y | | | | | | | Y |
| YP0371 | Y | | | | | | | | Y |

Legend for Table 3

| | |
|---|---|
| Fl | Flower |
| Si | Silique |
| Lf | Leaf |
| St | Stem |
| Em | Embryo |
| Ov | Ovule |
| Hy | Hypocotyl |
| Co | Cotyledon |
| Rt | Rosette |
| | Leaf |

The invention being thus described, it will be apparent to one of ordinary skill in the art that various modifications of the materials and methods for practicing the invention can be made. Such modifications are to be considered within the scope of the invention as defined by the following claims.

Each of the references from the patent and periodical literature cited herein is hereby expressly incorporated in its entirety by such citation.

What is claimed is:

1. An isolated nucleic acid molecule capable of modulating transcription wherein the nucleic acid molecule shows at least 80% sequence identity to one of the promoter sequences in Table 1, or a complement thereof.

2. The isolated nucleic acid molecule of claim 1, wherein said nucleic acid is capable of functioning as a promoter.

3. The isolated nucleic acid molecule of claim 2, wherein said nucleic acid comprises a reduced promoter nucleotide sequence having a sequence consisting of one of the promoter sequences in Table 1 having at least one of the corresponding optional promoter fragments identified in Table 1 deleted therefrom.

4. The isolated nucleic acid molecule of claim 2, wherein said nucleic acid comprises a reduced promoter nucleotide sequence having a sequence consisting of one of the promoter sequences in Table 1 having all of the corresponding optional promoter fragments identified in Table 1 deleted therefrom.

5. The isolated nucleic acid molecule of claim 1, wherein said nucleic acid molecule is capable of modulating transcription during the developmental times, or in response to a stimuli, or in a cell, tissue, or organ as set forth in Table 1 in the section “The spatial expression of the promoter-marker-vector”.

6. The isolated nucleic acid molecule according to claim 1, having a sequence according to any one of SEQ ID NO. 1 to 63.

7. A vector construct comprising:
a) a first nucleic acid capable of modulating transcription wherein the nucleic acid molecule shows at least 80% sequence identity tone of the promoter sequences in Table 1; and
b) a second nucleic acid having to be transcribed,
wherein said first and second nucleic acid molecules are heterologous to each other and are operably linked together.

8. The vector construct according to claim 7, wherein said nucleic acid comprises a reduced promoter nucleotide sequence having a sequence consisting of one of the promoter sequences in Table 1 having at least one of the corresponding optional promoter fragments identified in Table 1 deleted therefrom.

9. The vector construct according to claim 7, wherein said nucleic acid comprises a reduced promoter nucleotide sequence having a sequence consisting of one of the promoter sequences in Table 1 having all of the corresponding optional promoter fragments identified in Table 1 deleted therefrom.

10. A host cell comprising an isolated nucleic acid molecule according to claim 1, wherein said nucleic acid molecule is flanked by exogenous sequence.

11. The host cell according to claim 9, wherein said nucleic acid comprises a reduced promoter nucleotide sequence having a sequence consisting of one of the promoter sequences in Table 1 having at least one of the corresponding optional promoter fragments identified in Table 1 deleted therefrom.

12. The host cell according to claim 10, wherein said nucleic acid comprises a reduced promoter nucleotide sequence having a sequence consisting of one of the promoter sequences in Table 1 having all of the corresponding optional promoter fragments identified in Table 1 deleted therefrom.

13. A host cell comprising a vector construct of claim 7.

14. A method of modulating transcription by combining, in an environment suitable for transcription:
a) a first nucleic acid molecule capable of modulating transcription wherein the nucleic acid molecule shows at least 80% sequence identity to one of the promoter sequences in Table 1; and
b) a second molecule to be transcribed;
wherein the first and second nucleic acid molecules are heterologous to each other and operably linked together.

15. The method of claim 14, wherein said nucleic acid comprises a reduced promoter nucleotide sequence having a sequence consisting of one of the promoter sequences in Table 1 having at least one of the corresponding optional promoter fragments identified in Table 1 deleted therefrom.

16. The method of claim 14, wherein said nucleic acid comprises a reduced promoter nucleotide sequence having a sequence consisting of one of the promoter sequences in Table 1 having all of the corresponding optional promoter fragments identified in Table 1 deleted therefrom.

17. The method according to any one of claims 14-16, wherein said first nucleic acid molecule is capable of modulating transcription during the developmental times, or in response to a stimuli, or in a cell tissue, or organ as set forth in Table 1 in the section entitled “The spatial expression of the promoter-marker-vector” wherein said first nucleic acid molecule is inserted into a plant cell and said plant cell is regenerated into a plant.

18. A plant comprising a vector construct according to claim 7.

19. A transformed plant comprising a promoter according to claim 1, said transformed plant having characteristics which are different from those of a naturally occurring plant of the same species cultivated under the same conditions.

20. A seed of a plant according to claim 19.

21. A method of producing a transformed plant having characteristics different from those of a naturally occurring plant of the same species cultivated under the same conditions, which comprises introducing a promoter according to claim 1 into a plant to modulate transcription in a plant.
